SEQUENCE LISTING (73 sequences)

SEQ ID NO: 1 - DNA, 1100 bp, Homo sapiens
  CDS (147)..(1001)
aaattcaggataactctcctgaggggtgagccaagccctgccatgtagtgcacgcaggacatcaacaaacacagataacaggaaatgatccattccctgtggtcacttattctaaaggccccaaccttcaaagttcaagtagtgatatggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtgacctacttacaccatgtctgtagctattttcctccctttctctgtacctctaagaagaaagaatctaactgaaaataccaaaaaaaaaaaaaaaaaa

SEQ ID NO: 2 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 3 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 205 aa, Homo sapiens
MTPPERLFLPRVRGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 5 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 6 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 7 - DNA, 338 bp, Homo sapiens
  misc_feature (4)..(4): n equals a, t, g, or c
aggntaactctcctgaggggtgagccaagccctgccatgtagtgcacgcaggacatcancaaacacannnnncaggaaataatccattccctgtggtcacttattctaaaggccccaaccttcaaagttcaagtagtgatatggatgactccacagaaagggagcagtcacgccttacttcttgccttaagaaaagagaagaaatgaaactgnaaggagtgtgtttccatcctcccacggaaggaaagcccctctntccgatcctccaaagacggaaagctgctggctgcaaccttgntgntggcattgtgttcttgctgnctcaaggtggtgttntt

SEQ ID NO: 8 - DNA, 509 bp, Homo sapiens
  misc_feature (10)..(10): n equals a, t, g, or c
aattcggcanagnaaactggttacttttttatatatggtcaggttttatatactgataagacctacgccatgggacatctagttcagaggaagaaggtccatgtctttggggatgaattgagtctggtgactttgtttcgatgtattcaaaatatgcctgaaacactacccaataattcctgctattcagctggcattgcaaaactggnaggaaggagatgaactccaacttgcaataccaggggaaaatgcacaattatcactgggatggagatgttcacattttttgggtgccattgaaactgctgtgacctncttacancangtgctgttngctattttncctncctnttctntggtaacctcttaggaaggaaggattcttaactgggaaataacccaaaaaaannttaaangggtangngnnanangnggggnngttnncnngnngnnttttnggnntatnttntnntngggnnnngtaaaaatggggccnangggggnttttt

SEQ ID NO: 9 - DNA, 497 bp, Homo sapiens
  misc_feature (168)..(168): n equals a, t, g, or c
aattcggcacgagcaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgnttgcagacagtgaaacaccaactatacaaaaaggctcccttctgntgccacatttgggccaaggaatggagagatttcttcgtctggaaacattttgccaaactcttcagatactctttnctctctgggaatcaaaggaaaatctctacttagattnacacatttgttcccatgggtntcttaagttttaaaaggggagtgcccttaggaggaaaaggggataaatattggccaaggnactggttantttntaaatatggtcaggtttntatanctggtaggcctcgccatgggcattnattcanggngaggncnntcttttgggntga

SEQ ID NO: 10 - DNA, 27 bp, Artificial Sequence
  Neutrokine-alpha forward primer containing BamHI restriction site
gtgggatccagcctccgggcagagctg

SEQ ID NO: 11 - DNA, 33 bp, Artificial Sequence
  Neutrokine-alpha rev primer HindIII site and two stop codons
gtgaagcttttattacagcagtttcaatgcacc

SEQ ID NO: 12 - DNA, 26 bp, Artificial Sequence
  Neutrokine-alpha forward primer containing BspHI restriction site
gtgtcatgagcctccgggcagagctg

SEQ ID NO: 13 - DNA, 33 bp, Artificial Sequence; identical to SEQ ID NO: 11
  Neutrokine-alpha rev primer HindIII site and two stop codons

SEQ ID NO: 14 - DNA, 28 bp, Artificial Sequence
  Neutrokine-alpha forward primer containing BamHI restriction site
gtgggatccccgggcagagctgcagggc

SEQ ID NO: 15 - DNA, 33 bp, Artificial Sequence
  Neutrokine-alpha rev primer BamHI site and two stop codons
gtgggatccttattacagcagtttcaatgcacc

SEQ ID NO: 16 - DNA, 129 bp, Artificial Sequence
  Neutrokine-alpha fwd primer BamHI, Kozak, AUG, and IL-6
gcgggatccgccaccatgaactccttctccacaagcgccttcggtccagttgccttctccctggggctgctcctggtgttgcctgctgccttccctgccccagttgtgagacaaggggacctggccagc

SEQ ID NO: 17 - DNA, 30 bp, Artificial Sequence
  Neutrokine-alpha reverse primer containing BamHI site
gtgggatccttacagcagtttcaatgcacc

SEQ ID NO: 18 - DNA, 903 bp, Homo sapiens
  CDS (1)..(798)
atggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtgacctacttacaccatgtctgtagctattttcctccctttctctgtacctctaagaagaaagaatctaactgaaaataccaaaaaaaaaaaaaaaaaaaaaaaa

SEQ ID NO: 19 - protein, 266 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 20 - protein, 136 aa, Homo sapiens
HSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 21 - DNA, 462 bp, Homo sapiens
atggctgttcagggtccggaagaaaccgttactcaggactgccttcagctgatcgcagactctgaaactccgaccatccagaaaggttcttacacctttgttccttggctgctttctttcaaacgtggttctgccctggaagagaaagaaaacaaaatcctggttaaagaaactggttacttctttatctacggtcaggttctttacactgataagacctacgccatgggtcacctgattcagcgtaagaaagttcacgttttcggtgacgagctgtctctggttactctgtttcgctgcattcagaacatgccggaaactcttcctaacaactcctgctactctgctggcatcgcaaaactggaagagggtgatgaactgcagctggcaattcctcgtgaaaacgcacaaatttctctggacggtgatgtaaccttctttggtgcactgaaacttctgtaa

SEQ ID NO: 22 - DNA, 1040 bp, Mus musculus
  CDS (1)..(468)
cgcgtggtagacctctcagctcctcctgcaccatgcctgcctggatgccgccattctcaacatgatgataatggaatgaacctcagaaacagaacttacacatttgttccatggcttctcagctttaaaagaggaaatgccttggaggagaaagagaacaaaatagtggtgaggcaaacaggctatttcttcatctacagccaggttctatacacggaccccatctttgctatgggtcatgtcatccagaggaagaaagtacacgtctttggggacgagctgagcctggtgaccctgttccgatgtattcagaatatgcccaaaacactgcccaacaattcctgctactcggctggcatcgcgaggctggaagaaggagatgagattcagcttgcaattcctcgggagaatgcacagatttcacgcaacggagacgacaccttctttggtgccctaaaactgctgtaactcacttgctggagtgcgtgatccccttccctcgtcttctctgtacctccgagggagaaacagacgactggaaaaactaaaagatggggaaagccgtcagcgaaagttttctcgtgacccgttgaatctgatccaaaccaggaaatataacagacagccacaaccgaagtgtgccatgtgagttatgagaaacggagcccgcgctcagaaagaccggatgaggaagaccgttttctccagtcctttgccaacacgcaccgcaaccttgctttttgccttgggtgacacatgttcagaatgcagggagatttccttgttttgcgatttgccatgagaagagggcccacaactgcaggtcactgaagcattcacgctaagtctcaggatttactctcccttctcatgctaagtacacacacgctcttttccaggtaatactatgggatactatggaaaggttgtttgtttttaaatctagaagtcttgaactggcaatagacaaaaatccttataaattcaagtgtaaaataaacttaattaaaaaggtttaagtgtgaaaaaaaaaaaaaaaa

SEQ ID NO: 23 - protein, 155 aa, Mus musculus
RVVDLSAPPAPCLPGCRHSQHDDNGMNLRNRTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 24 - DNA, 26 bp, Artificial Sequence
  Neutrokine-alpha primer
ccaccagctccaggagaaggcaactc

SEQ ID NO: 25 - DNA, 19 bp, Artificial Sequence
  Neutrokine-alpha probe
accgcgggactgaaaatct

SEQ ID NO: 26 - DNA, 23 bp, Artificial Sequence
  Neutrokine-alpha primer
cacgcttatttctgctgttctga

SEQ ID NO: 27 - DNA, 657 bp, Macaca irus
taccaggtggcggccgtgcaaggggacctggccagcctccgggcagagctgcagggccaccacgcggagaagctgccagcaagagcaagagcccccaaggccggtctgggggaagctccagctgtcaccgcaggactgaaaatctttgaaccaccagctccaggagaaggcaactccagtcagagcagcagaaataagcgtgctattcagggtgcagaagaaacagtcattcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatacactgataagacctatgccatgggacatctaattcagaggaaaaaagtccatgtctttggggatgaattgagtctggtgactttgtttcgatgtattcaaaatatgcctgaaacactacccaataattcctgctattcagctggcattgcaaaactggaagaaggagatgaacttcaacttgcaataccacgagaaaatgcacaaatatcactggatggagatgtcacattttttggtgccctcaaactgctg

SEQ ID NO: 28 - protein, 219 aa, Macaca irus
YQVAAVQGDLASLRAELQGHHAEKLPARARAPKAGLGEAPAVTAGLKIFEPPAPGEGNSSQSSRNKRAIQGAEETVIQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 29 - DNA, 657 bp, Macaca mulatta (Rhesus Monkey)
taccaggtggcggccgtgcaaggggacctggccagcctccgggcagagctgcagagccaccacgcggagaagctgccagcaagagcaagagcccccaaggccggtctgggggaagctccagctgtcaccgcgggactgaaaatctttgaaccaccagctccaggagaaggcaactccagtcagagcagcagaaataagcgtgctattcagggtgcagaagaaacagtcattcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatacactgataagacctatgccatgggacatctaattcagaggaaaaaagtccatgtctttggggatgaattgagtctggtgactttgtttcgatgtattcaaaatatgcctgaaacactacccaataattcctgctattcagctggcattgcaaaactggaagaaggggatgaacttcaacttgcaataccacgagaaaatgcacaaatatcactggatggagatgtcacattttttggtgccctcaaactgctg

SEQ ID NO: 30 - protein, 219 aa, Macaca mulatta (Rhesus Monkey)
YQVAAVQGDLASLRAELQSHHAEKLPARARAPKAGLGEAPAVTAGLKIFEPPAPGEGNSSQSSRNKRAIQGAEETVIQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 31 - DNA, 38 bp, Artificial Sequence
  Neutrokine-alpha fwd primer PSC signal peptide C-terminus
ggtcgccgtttctaacgcggccgttcagggtccagaag

SEQ ID NO: 32 - DNA, 49 bp, Artificial Sequence
  Rev primer pA2GP vector and KpnI restriction site
ctggttcggcccaaggtaccaagcttgtaccttagatcttttctagatc

SEQ ID NO: 33 - DNA, 21 bp, Artificial Sequence
  Fwd primer for PSC baculovirus transfer plasmid pMGS12
ctggtagttcttcggagtgtg

SEQ ID NO: 34 - DNA, 19 bp, Artificial Sequence
  Rev primer for PSC baculovirus transfer plasmid pMGS12
cgcgttagaaacggcgacc

SEQ ID NO: 35 - DNA, 22 bp, Artificial Sequence
  Neutrokine-alpha degenerate oligonucleotide forward primer
taccagntggcngccntgcaag

SEQ ID NO: 36 - DNA, 22 bp, Artificial Sequence
  Neutrokine-alpha degenerate oligonucleotide reverse primer
gtnacagcagtttnanngcacc

SEQ ID NO: 37 - DNA, 867 bp, Mus musculus
atggatgagtctgcaaagaccctgccaccaccgtgcctctgtttttgctccgagaaaggagaagatatgaaagtgggatatgatcccatcactccgcagaaggaggagggtgcctggtttgggatctgcagggatggaaggctgctggctgctaccctcctgctggccctgttgtccagcagtttcacagcgatgtccttgtaccagttggctgccttgcaagcagacctgatgaacctgcgcatggagctgcagagctaccgaggttcagcaacaccagccgccgcgggtgctccagagttgaccgctggagtcaaactcctgacaccggcagctcctcgaccccacaactccagccgcggccacaggaacagacgcgccttccagggaccagaggaaacagaacaagatgtagacctctcagctcctcctgcaccatgcctgcctggatgccgccattctcaacatgatgataatggaatgaacctcagaaacatcattcaagactgtctgcagctgattgcagacagcgacacgccggccttggaggagaaagagaacaaaatagtggtgaggcaaacaggctatttcttcatctacagccaggttctatacacggaccccatctttgctatgggtcatgtcatccagaggaagaaagtacacgtctttggggacgagctgagcctggtgaccctgttccgatgtattcagaatatgcccaaaacactgcccaacaattcctgctactcggctggcatcgcgaggctggaagaaggagatgagattcagcttgcaattcctcgggagaatgcacagatttcacgcaacggagacgacaccttctttggtgccctaaaactgctg

SEQ ID NO: 38 - protein, 289 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 39 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 40 - protein, 290 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNRTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 41 - protein, 152 aa, Rattus sp
AFQGPEETVIQDCLQLIADSNTPTIRKGTYTFVPWLLSFKRGNALEEKENKIVVRQTGYFFIYSQVLYTDPIFAMGHVIQRKKIHVFGDELSLVTLFRCIQNMPKTLPNNSCYSAGIAKLEEGDEVQLAIPRENAQISRNGDDTFFGALKLL

SEQ ID NO: 42 - protein, 165 aa, Rattus sp
AFQGPEETEQDVDLSATPVPSLPGNCHASHHDENGLNLRTRTYTFVPWLLSFKRGNALEEKENKIVVRQTGYFFIYSQVLYTDPIFAMGHVIQRKKIHVFGDELSLVTLFRCIQNMPKTLPNNSCYSAGIAKLEEGDEIQLAIPRENAQISRNGDDTFFGALKLL

SEQ ID NO: 43 - protein, 184 aa, Rattus sp
AFQGPEETEQDVDLSATPAPSLPGNCHASHHDENGLNLRTIIQDCLQLIADSNTPTIRKGTYTFVPWLLSFKRGNALEEKENKIVVRQTGYFFIYSQVLYTDPIFAMGHVIQRKKIHVFGDELSLVTLFRCIQNMPKTLPNNSCYSAGIAKLEEGDEIQLAIPRENAQISRNGDDTFFGALKLL

SEQ ID NO: 44 - protein, 133 aa, Rattus sp
AFQGPEETGTYTFVPWLLSFKRGNALEEKENKIVVRQTGYFFIYSQVLYTDPIFAMGHVIQRKKIHVFGDELSLVTLFRCIQNMPKTLPNNSCYSAGIAKLEEGDEIQLAIPRENAQISRNGDDTFFGALKLL

SEQ ID NO: 45 - protein, 17 aa, Homo sapiens
MLQNSAVLLLLVISASA

SEQ ID NO: 46 - protein, 22 aa, Artificial Sequence
  consensus signal sequence
MPTWAWWLFLVLLLALWAPARG

SEQ ID NO: 47 - protein, 250 aa, Homo sapiens [sequence omitted]

SEQ ID NO: 48 - DNA, 38 bp, Artificial Sequence
  Oligonucleotide primer
cagactggatccgccaccatggatgactccacagaaag

SEQ ID NO: 49 - DNA, 33 bp, Artificial Sequence
  Oligonucleotide Primer
cagactggtaccgtcctgcgtgcactacatggc

SEQ ID NO: 50 - DNA, 21 bp, Artificial Sequence
  Oligonucleotide Primer
tggtgtctttctaccaggtgg

SEQ ID NO: 51 - DNA, 21 bp, Artificial Sequence
  Oligonucleotide primer
tttcttctggaccctgaacgg

SEQ ID NO: 52 - DNA, 4458 bp, Artificial
  pML124
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagtacgcaagttcacgtaaaggaagtatctcatatgcagatcttcgtgtaaaccggtaccggcaaaaccatcactctagaagtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 53 - DNA, 4980 bp, Artificial Sequence
  pML124MBPss-BLyS
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagtacgcaagttcacgtaaaggaagtatctcatatgaaaataaaaacaggtgcacgcatcctcgcattatccgcattaacgacgatgatgttttccgcctcggctctcgccgctgttcagggtccggaagaaaccgttactcaggactgccttcagctgatcgcagactctgaaactccgaccatccagaaaggttcttacacctttgttccttggctgctttctttcaaacgtggttctgccctggaagagaaagaaaacaaaatcctggttaaagaaactggttacttctttatctacggtcaggttctttacactgataagacctacgccatgggtcacctgattcagcgtaagaaagttcacgttttcggtgacgagctgtctctggttactctgtttcgctgcattcagaacatgccggaaactcttcctaacaactcctgctactctgctggcatcgcaaaactggaagagggtgatgaactgcagctggcaattcctcgtgaaaacgcacaaatttctctggacggtgatgtaaccttctttggtgcactgaaacttctgtaataataaggtaccggcaaaaccatcactctagaagtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 54 - protein, 26 aa, Artificial Sequence
  MBP signal sequence
MKIKTGARILALSALTTMMFSASALA

SEQ ID NO: 55 - protein, 178 aa, Artificial Sequence
  MBPss-Neutrokine-alpha
MKIKTGARILALSALTTMMFSASALAAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 56 - DNA, 21 bp, Artificial Sequence
  wild-type Shine Dalgarno box and its adjacent sequence
tttgtacatggagaaaataaa

SEQ ID NO: 57 - DNA, 21 bp, Artificial Sequence
  optimized Shine Dalgarno box and its adjacent sequence
cacgtaaaggaagtatctcat

SEQ ID NO: 58 - protein, 120 aa, Mus musculus
QVQLQQSGAELARPGASVKMSCRASGYTFTTYTMHWVKQRPGQGLEWIGYIIPRNTYTTFNQKFKNKATLTADKSSNTAYMQLSSLTSEDSAVYYCARHYGGGYWFFDVWGAGTTVTVSS

SEQ ID NO: 59 - protein, 113 aa, Mus musculus
ELVMTQTPASLAVSLGQRATISCRGSESVDSYGNSFMHWYQQKPGQPPKLLIYRASNLESGIPARFSGSGSRTDFTLTIYPVEADDVATYYCQQSNDDPMYTFGGGTKLEIKR

SEQ ID NO: 60 - protein, 249 aa, Homo sapiens
QVQLQQSGAEVKKPGSSVRVSCKASGGTFNNNAINWVRQAPGQGLEWMGGIIPMFGTAKYSQNFQGRVAITADESTSTASMELSSLRSEDTAVYYCARSRDLLLFPHYGMDVWGRGTMVTVSSGGGGSGGGGSGGGGSAFSSELTQDPAVSVALGQTVRVTCQGDSLRSYYASWYQQKPGQAPVLVIYGKNNRPSGIPDRFSGSSSGNTASLTITGAQAEDEADYYCNSRDSSGNHWVFGGGTELTVLG

SEQ ID NO: 61 - protein, 249 aa, Homo sapiens
QVQLQQSGAEVKKPGSSVRVSCKASGGTFNNNAINWVRQAPGQGLEWMGGIIPMFGTAKYSQNFQGRVAITADESTGTASMELSSLRSEDTAVYYCARSRDLLLFPHHALSPWGRGTMVTVSSGGGGSGGGGSGGGGSAFSSELTQDPAVSVALGQTVRVTCQGDSLRSYYASWYQQKPGQAPVLVIYGKNNRPSGIPDRFSGSSSGNTASLTITGAQAEDEADYYCSSRDSSGNHWVFGGGTELTVLG

SEQ ID NO: 62 - DNA, 882 bp, Homo sapiens
  CDS (1)..(882)
atgagtggcctgggccggagcaggcgaggtggccggagccgtgtggaccaggaggagcgctttccacagggcctgtggacgggggtggctatgagatcctgccccgaagagcagtactgggatcctctgctgggtacctgcatgtcctgcaaaaccatttgcaaccatcagagccagcgcacctgtgcagccttctgcaggtcactcagctgccgcaaggagcaaggcaagttctatgaccatctcctgagggactgcatcagctgtgcctccatctgtggacagcaccctaagcaatgtgcatacttctgtgagaacaagctcaggagcccagtgaaccttccaccagagctcaggagacagcggagtggagaagttgaaaacaattcagacaactcgggaaggtaccaaggattggagcacagaggctcagaagcaagtccagctctcccggggctgaagctgagtgcagatcaggtggccctggtctacagcacgctggggctctgcctgtgtgccgtcctctgctgcttcctggtggcggtggcctgcttcctcaagaagaggggggatccctgctcctgccagccccgctcaaggccccgtcaaagtccggccaagtcttcccaggatcacgcgatggaagccggcagccctgtgagcacatcccccgagccagtggagacctgcagcttctgcttccctgagtgcagggcgcccacgcaggagagcgcagtcacgcctgggacccccgaccccacttgtgctggaaggtgggggtgccacaccaggaccacagtcctgcagccttgcccacacatcccagacagtggccttggcattgtgtgtgtgcctgcccaggaggggggcccaggtgcataa

SEQ ID NO: 63 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 64 - DNA, 834 bp, Homo sapiens
  CDS (67)..(621)
ttgtaagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataagctctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttctatgattaaagtctttt

SEQ ID NO: 65 - protein, 184 aa, Homo sapiens [sequence omitted]

SEQ ID NO: 66 - DNA, 899 bp, Homo sapiens
  CDS (6)..(560)
gcaccatgaggcgagggccccggagcctgcggggcagggacgcgccagcccccacgccctgcgtcccggccgagtgcttcgacctgctggtccgccactgcgtggcctgcgggctcctgcgcacgccgcggccgaaaccggccggggccagcagccctgcgcccaggacggcgctgcagccgcaggagtcggtgggcgcgggggccggcgaggcggcgctgcccctgcccgggctgctctttggcgcccccgcgctgctgggcctggcactggtcctggcgctggtcctggtgggtctggtgagctggaggcggcgacagcggcggcttcgcggcgcgtcctccgcagaggcccccgacggagacaaggacgccccagagcccctggacaaggtcatcattctgtctccgggaatctctgatgccacagctcctgcctggcctcctcctggggaagacccaggaaccaccccacctggccacagtgtccctgtgccagccacagagctgggctccactgaactggtgaccaccaagacggccggccctgagcaacaatagcagggagccggcaggaggtggcccctgccctccctctggacccccagccaggggcttggaaatcaaattcagctcttcactccagcatgcacatgccctctttctgggaccaggctaaccctgcagaagcacagacactacagaccacagcattcagcccccatggagtttggtgtgcttgcctttggcttcagacctcaccatctttgacagcccttgaaggtggtagcccagctcctgttcctgtgccttcaaaaggctggggcactatgagtaaaagaccgcttttaaaatggggaaggcaccattaagccaaaatgaatctgaaaaaagacaaaa

SEQ ID NO: 67 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 68 - protein, 293 aa, Artificial
  Neutrokine-alpha inhibitory peptibody
MLPGCKWDLLIKQWVCDPLGSGSATGGSGSTASSGSGSATHMLPGCKWDLLIKQWVCDPLGGGGGVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 293 aa, Artificial
  Neutrokine-alpha inhibitory peptibody
MFHDCKWDLLTKQWVCHGLGSGSATGGSGSTASSGSGSATHMFHDCKWDLLTKQWVCHGLGGGGGVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 18 aa, Artificial
  Neutrokine-alpha binding peptide
GQMGWRWDPLTKMWLGTS

SEQ ID NO: 71 - protein, 70 aa, Artificial
  Amino acids 1-70 of BAFF-R with V20N & L27P substitutions
MRRGPRSLRGRDAPAPTPCNPAECFDPLVRHCVACGLLRTPRPKPAGASSPAPRTALQPQESVGAGAGEA

SEQ ID NO: 72 - protein, 249 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGPRYYDILTGYRYNWFDPWGRGTLVTVSSGGGGSGGGGSGGGGSDIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIKR

SEQ ID NO: 73 - protein, 251 aa, Homo sapiens
QLQLQESGPGLVKPSETLSLTCTVSGGFISSRTSYWGWIRQPPGKGPEWIGNIYYTGKTYYSPSLKSRVTISVDTSKNQLSLKLNSVTAADTAVYYCARAGYDLLTGYPFYFDSWGKGTLVTVSSGGGGSGGGGSGGGGSALEIVLTQSPATLSLSPGERATLSCRASQSVSSYLAWYQQKPGQAPRLLIYDASNRATGIPARFSGSGSGTDFTLTISSLEPEDFAVYYCQQRSNWPFLTFGGGTKVEIKR

(19) United States
(12) Patent Application Publication
(10) Pub. No.: US 2011/0311548 A1
(43) Pub. Date: Dec. 22, 2011

(54) USE OF B LYMPHOCYTE STIMULATOR PROTEIN ANTAGONISTS TO TREAT ASTHMA AND OTHER ALLERGIC AND INFLAMMATORY CONDITIONS OF THE RESPIRATORY SYSTEM

(75) Inventors: Heather Wasserman, Gaithersburg, MD (US); Timothy L. Carlson, Silver Spring, MD (US); Thi-Sau Migone, Gaithersburg, MD (US); Madhav Devalaraja, Rockville, MD (US)

(73) Assignee: HUMAN GENOME SCIENCES, INC., Rockville, MD (US)

(21) Appl. No.: 13/163,593

(22) Filed: Jun. 17, 2011

Related U.S. Application Data
(60) Provisional application No. 61/488,730, filed on May 21, 2011.
(60) Provisional application No. 61/389,018, filed on Oct. 1, 2010.
(60) Provisional application No. 61/356,485, filed on Jun. 18, 2010.

(51) Int. Cl. A61K 39/395; A61P 11/06; A61P 11/00; A61K 38/16; A61K 39/35

(52) U.S. Cl. 424/145.1; 424/158.1; 514/1.7; 424/275.1

(57) ABSTRACT

The invention relates to methods of preventing, treating, and ameliorating asthma, allergen-induced respiratory symptoms, and/or an allergic or inflammatory condition of the lung or respiratory system in a patient by administering B Lymphocyte Stimulator antagonists. In addition, the invention provides a method of reducing total serum IgE levels in a patient suffering from asthma, and/or an allergic or inflammatory condition of the lung or respiratory system by administering B Lymphocyte Stimulator antagonists. The invention further provides a method of treating, preventing or ameliorating an allergen-induced airway hyper responsiveness or increase in mucus-containing cells and/or mucus accumulation or production in the airway epithelium of a patient by administering B Lymphocyte Stimulator antagonist.

### CROSS-REFERENCE TO RELATED APPLICATIONS

This patent application claims the benefit of U.S. Provisional Patent Application No. 61/356,485, filed Jun. 18, 2010; U.S. Provisional Patent Application No. 61/389,018, filed Oct. 1, 2010; and U.S. Provisional Application No. 61/488,730, filed May 21, 2011, which are incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 113,726 Byte ASCII (Text) file named “SEQUENCELISTING.TXT,” created on Jun. 17, 2011.

### BACKGROUND OF THE INVENTION

Asthma is a chronic inflammatory condition of the lungs that affects 7% of the population worldwide. Asthma is characterized by recurrent episodes of respiratory symptoms, including wheezing, chest tightness, shortness of breath, and coughing; variable airflow obstruction; presence of airway hyper reactivity; and chronic airway inflammation. Asthma affects people of all ages, but it most often starts in childhood. In the United States, more than 22 million people are known to have asthma and nearly 6 million of these people are children.

Although progress has been made in understanding the mechanisms underlying the pathogenesis of asthma, there is no cure for asthma. Current asthma treatments are directed at controlling the symptoms of asthma and are primarily directed towards suppressing airway inflammation and relieving bronchoconstriction. All of these therapies exert their effect downstream of the origins of asthma. For example, common asthma treatments include the use of long-term control medications to prevent flare-ups in combination with a quick-relief inhaler to control symptoms once they start. Corticosteroids are the most commonly prescribed type of long-term asthma medication. However, there are many side effects associated with the long-term use of corticosteroids, including an increased risk for cataracts, osteoporosis, and infection; suppression of adrenal gland hormone production; and poor wound healing. Thus, it is desirable to target an upstream mediator of asthma in order to prevent the symptoms of asthma and avoid the need for long-term corticosteroid treatment.

A variety of cell types, including mast cells, eosinophils, T lymphocytes, macrophages, neutrophils, and epithelial cells, have been shown to play an important role in the pathogenesis of asthma and other allergic and inflammatory conditions of the respiratory system, such as chronic rhinosinusitis. In addition, B-cells have been implicated as an upstream mediator of the allergic response, causing disease via IgE production and subsequent basophil and mast cell activation and degranulation.

In view of the foregoing, there is a need for methods of treating, preventing and/or ameliorating asthma and other allergic and inflammatory conditions of the respiratory system by targeting B-cells.

### SUMMARY OF THE INVENTION

The invention provides methods of using B Lymphocyte Stimulator antagonists to treat, prevent, and/or ameliorate asthma, allergen-induced respiratory symptoms, and other allergic and inflammatory conditions of the respiratory system (e.g., the nostrils, nasopharynx, pharynx, trachea, bronchiole, bronchus, lungs, and/or alveoli).

One embodiment of the invention provides a method of treating, preventing or ameliorating asthma in a patient comprising administering to the patient an effective amount of a B Lymphocyte Stimulator antagonist, thereby treating, preventing or ameliorating asthma in the patient.

Another embodiment of the invention provides a method of treating, preventing or ameliorating an allergic condition of the lung or respiratory system, such as extrinsic allergic alveolitis (hypersensitivity pneumonitis) and eosinophilic lung disorders, including allergic bronchopulmonary aspergillosis, acute and chronic eosinophilic pneumonia, Churg-Strauss syndrome, and idiopathic hypereosinophilic syndrome, in a patient comprising administering to the patient an effective amount of a B Lymphocyte Stimulator antagonist, thereby treating, preventing or ameliorating the allergic condition of the lung or respiratory system in the patient. The allergic condition of the lung or respiratory system can be characterized by an increase in allergen-induced airway hyper responsiveness and/or by an increase in mucus-containing cells. In addition, the allergic condition of the lung or respiratory system can be characterized by an increase in cellular infiltration of cells of the immune system, such as B cells, mast cells, or other leukocytes and lymphocytes.

Another embodiment of the invention provides a method of treating, preventing or ameliorating an inflammatory condition of the respiratory system (e.g., the nostrils, nasopharynx, pharynx, trachea, bronchiole, bronchus, lungs, and/or alveoli), such as chronic rhinosinusitis, in a patient comprising administering to the patient an effective amount of a B Lymphocyte Stimulator antagonist, thereby treating, preventing or ameliorating the allergic inflammatory condition of the respiratory system in the patient.

Another embodiment of the invention provides a method of treating, preventing or ameliorating an inflammatory condition of the lung in a patient comprising administering to the patient an effective amount of a B Lymphocyte Stimulator antagonist, thereby decreasing, treating, preventing or ameliorating lung inflammation in the patient.

The inflammatory condition of the lung or respiratory system can be characterized by an increase in allergen-induced airway hyper responsiveness and/or by an increase in mucus-containing cells. In addition, the inflammatory condition of the lung or respiratory system can be characterized by an increase in cellular infiltration of cells of the immune system such as B cells, mast cells or other leukocytes and lymphocytes.

Another embodiment of the invention provides a method of reducing total serum IgE levels in a patient suffering from asthma or an allergic or inflammatory condition of the nostrils, nasopharynx, pharynx, trachea, bronchiole, bronchus, lungs, and/or alveoli comprising administering to the patient an effective amount of a B Lymphocyte Stimulator antagonist, thereby reducing total serum IgE levels in the patient. In another embodiment, the invention provides a method of reducing an allergen-induced increase in IgE levels in a patient suffering from asthma or an allergic or inflammatory condition of the nostrils, nasopharynx, pharynx, trachea, bronchiole, bronchus, lungs, and/or alveoli comprising administering to the patient an effective amount of a B Lymphocyte Stimulator antagonist, thereby reducing the allergen-induced increase in IgE levels in the patient.

Another embodiment of the invention provides a method of preventing allergen-induced airway hyper responsiveness in a patient comprising administering to the patient an effective amount of a B Lymphocyte Stimulator antagonist, thereby preventing allergen-induced airway hyper responsiveness in the patient.

Another embodiment of the invention provides a method of preventing an allergen-induced increase in mucus-containing cells in the airway epithelium of a patient comprising administering to the patient an effective amount of a B Lymphocyte Stimulator antagonist, thereby preventing an allergen-induced increase in mucus-containing cells in the airway epithelium of the patient.

Another embodiment of the invention provides a method of treating or ameliorating allergen-induced airway hyper responsiveness in a patient comprising administering to the patient an effective amount of a B Lymphocyte Stimulator antagonist, thereby inhibiting allergen-induced airway hyper responsiveness in the patient.

Another embodiment of the invention provides a method of treating or ameliorating allergen-induced increase in mucus-containing cells in the airway epithelium of a patient comprising administering to the patient an effective amount of a B Lymphocyte Stimulator antagonist, thereby inhibiting allergen-induced increase in mucus-containing cells in the airway epithelium of the patient.

Preferred antagonists for use in the invention are antibodies that bind to B Lymphocyte Stimulator protein. Additional B Lymphocyte Stimulator antagonists for use in the invention include: a protein comprising the B Lymphocyte Stimulator binding domain of transmembrane activator and CAML interactor (TALI); a protein comprising the B Lymphocyte Stimulator binding domain of B-cell maturation antigen (BCMA); a protein comprising the B Lymphocyte Stimulator binding domain of B cell activating factor receptor (BAFF-R); a B Lymphocyte Stimulator binding peptide or polypeptide; a B Lymphocyte Stimulator peptibody; a B Lymphocyte Stimulator protein variant; and an anti-B Lymphocyte Stimulator receptor antibody.

### BRIEF DESCRIPTION OF THE FIGURES

The patent or application file contains at least one drawing executed in color. Copies of this patent or patent application publication with color drawings will be provided by the Office upon request and payment of the necessary fee.

The following drawings are illustrative of embodiments of the invention and are not meant to limit the scope of the invention as encompassed by the claims.

FIGS. 1A-D are a series of graphs illustrating the effects of prophylactic and therapeutic administration of anti-BLyS antibody in a murine model of asthma. Female BALB/c mice (8-10 wk of age) were sensitized with intraperitoneal (i.p.) injections of 20 μg ovalbumin (OVA) (0.1 ml of 200 μg/ml in PBS) every other day from day 1 through 19. Mice were challenged by nebulization with 10 mg/ml OVA in PBS for 40 minutes on days 33, 35, 37, and 39. One group of mice, labeled 10F4 prophylactic (Pro.), was treated with 100 μg of monoclonal IgG1 hamster anti-mouse BLyS (clone 10F4) by i.p. injection on days 20 and 27, prior to challenge with OVA (prophylactic group). Another group of mice, labeled 10F4 therapeutic (Ther.), was treated with 100 μg of monoclonal IgG1 hamster anti-mouse BLyS (clone 10F4) by i.p. injection on day 34 (therapeutic group). Controls included (1) a group of mice (Iso. Cont.) treated with 100 μg of an isotype control antibody (purified Armenian hamster IgG1 (clone G235-2356; BD Pharmingen)) by i.p. injection on day 34; (2) a group of mice (Dex) treated with 50 μg of water-soluble dexamethasone in 100 μl of PBS by i.p. injection on days 34, 36, and 38 (i.e., every other day of the OVA challenge); (3) a group of mice (PBS) sensitized and challenged with PBS; and (4) a group of mice (Naïve) left untreated. All mice were terminated on day 40. FIG. 1A is a graph illustrating the total ng/ml of IgE (y-axis) for each treatment group (x-axis). FIG. 1B is a graph illustrating the percent of B220+ B cells in the spleen (y-axis) for each treatment group (x-axis). FIG. 1C is a graph illustrating the percent of B220+ B cells in the blood (y-axis) for each treatment group (x-axis). FIG. 1D is a graph illustrating the total ng/ml of BLyS protein in the serum (y-axis) for each treatment group (x-axis).

FIGS. 2A-D are a series of graphs illustrating the effects of administration of anti-BLyS antibody in a murine model of asthma. Female BALB/c mice (8-10 wk of age) were sensitized with intraperitoneal (i.p.) injections of 20 μg OVA (0.1 ml of 200 m/ml in PBS) every other day from day 1 through 19. Mice were challenged by nebulization with 10 mg/ml OVA in PBS for 40 minutes on days 33, 35, 37, 39, 47, 49, 51, and 53. Mice were treated with either 100 μg of monoclonal IgG1 hamster anti-mouse BLyS (clone 10F4) or an isotype control antibody by i.p. injection on days 34, 41, and 48. All mice were terminated on day 54. FIG. 2A is a graph illustrating the total ng/ml of IgE (y-axis) for each treatment group (x-axis). FIG. 2B is a graph illustrating the percent of B220+ B cells in the spleen (y-axis) for each treatment group (x-axis). FIG. 2C is a graph illustrating the percent of B220+ B cells in the blood (y-axis) for each treatment group (x-axis). FIG. 2D is a graph illustrating the total ng/ml of BLyS protein in the serum (y-axis) for each treatment group (x-axis).

FIGS. 3A-D are a series of graphs illustrating the effects of administration of anti-BLyS antibody in a murine model of asthma. Female BALB/c mice (8-10 wk of age) were sensitized with intraperitoneal (i.p.) injections of 20 μg OVA (0.1 ml of 200 m/ml in PBS) every other day from day 1 through 19. Mice were challenged by nebulization with 10 mg/ml OVA in PBS for 40 minutes on days 33, 35, 37, 39, 47, 49, 51, 53, 61, 63, 65, and 67. Mice were treated with either 100 μg of monoclonal IgG1 hamster anti-mouse BLyS (clone 10F4) or an isotype control antibody by i.p. injection on days 34, 41, 48, and 55. All mice were terminated on day 68. FIG. 3A is a graph illustrating the total ng/ml of IgE (y-axis) for each treatment group (x-axis). FIG. 3B is a graph illustrating the percent of B220+ B cells in the spleen (y-axis) for each treatment group (x-axis). FIG. 3C is a graph illustrating the percent of B220+ B cells in the blood (y-axis) for each treatment group (x-axis). FIG. 3D is a graph illustrating the total ng/ml of BLyS protein in the serum (y-axis) for each treatment group (x-axis).

FIGS. 4A-C are a series of graphs illustrating the long-term effects of administration of anti-BLyS antibody in a murine model of asthma. Female BALB/c mice (8-10 wk of age) were sensitized with intraperitoneal (i.p.) injections of 20 μg OVA (0.1 ml of 200 μg/ml in PBS) every other day from day 1 through 19. Mice were challenged by nebulization with 10 mg/ml OVA in PBS for 40 minutes on days 33, 35, 37, 39, 47, 49, 51, 53, 61, 63, 65, 67, 120, 122, 124, and 126. Mice were treated with either 100 μg of monoclonal IgG1 hamster anti-mouse BLyS (clone 10F4) or an isotype control antibody by i.p. injection on days 34, 41, 48, and 55. As a control, mice were either sensitized and challenged with PBS or left untreated (Naïve). All mice were terminated on day 127. FIG. 4A is a graph illustrating the total ng/ml of IgE (y-axis) for each treatment group (x-axis). FIG. 4B is a graph illustrating the percent of B220+ B cells in the spleen (y-axis) for each treatment group (x-axis). FIG. 4C is a graph illustrating the percent of B220+ B cells in the blood (y-axis) for each treatment group (x-axis).

FIGS. 5A-C are a series of graphs illustrating the long-term effects of administration of anti-BLyS antibody in a murine model of asthma. Female BALB/c mice (8-10 wk of age) were sensitized with intraperitoneal (i.p.) injections of 20 μg OVA (0.1 ml of 200 μg/ml in PBS) every other day from day 1 through 19. Mice were challenged by nebulization with 10 mg/ml OVA in PBS for 40 minutes on days 33, 35, 37, 39, 47, 49, 51, 53, 61, 63, 65, 67, 120, 122, 124, 126, 152, 154, 156, and 158. As a control, one group of mice, labeled No Rx, was left untreated. One group of mice, labeled 2× 10F4, was treated with 100 μg of monoclonal IgG1 hamster anti-mouse BLyS (clone 10F4) by i.p. injection on days 34 and 41. One group of mice, labeled 4× 10F4, was treated with 100 μg of monoclonal IgG1 hamster anti-mouse BLyS (clone 10F4) by i.p. injection on days 34, 41, 48, and 55. All mice were terminated on day 159. FIG. 5A is a graph illustrating the total ng/ml of IgE (y-axis) for each treatment group (x-axis). FIG. 5B is a graph illustrating the percent of B220+ B cells in the spleen (y-axis) for each treatment group (x-axis). FIG. 5C is a graph illustrating the percent of B220+ B cells in the blood (y-axis) for each treatment group α-axis).

FIG. 6 depicts photographs of lung sections from a murine model of asthma. OVA-sensitized and challenged mice were treated twice with either 100 μg of monoclonal anti-BLyS antibody 10F4 or an isotype control antibody as described in FIG. 1. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 7 depicts photographs of lung sections from a murine model of asthma. OVA-sensitized and challenged mice were treated three times with either 100 μg of monoclonal IgG1 hamster anti-mouse BLyS (clone 10F4) or an isotype control antibody as described in FIG. 2. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 8 depicts photographs of lung sections from a murine model of asthma. OVA-sensitized and challenged mice were treated four times with either 100 μg of monoclonal IgG1 hamster anti-mouse BLyS (clone 10F4) or an isotype control antibody as described in FIG. 3. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 9 depicts photographs of lung sections from a murine model of asthma. OVA-sensitized and challenged mice were treated four times with either 100 μg of monoclonal IgG1 hamster anti-mouse BLyS (clone 10F4) or an isotype control antibody as described in FIG. 4. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 10 is a graph illustrating the effects of prophylactic and therapeutic administration of an anti-BLyS antibody on OVA-specific IgE levels in a murine model of asthma. Female BALB/c mice (8-10 wk of age) were treated as described in FIG. 1 and OVA-specific IgE levels were detected in serum isolated from each of the treatment groups. FIG. 10 is a graph illustrating the pg/ml of OVA-specific IgE detected in the serum (y-axis) for each treatment group (x-axis).

FIG. 11 depicts photographs of lung sections from a murine model of asthma. OVA-sensitized and challenged mice were treated on day 34 with either 100 μg of monoclonal IgG1 hamster anti-mouse BLyS (clone 10F4) (labeled 10F4 therapeutic) or an isotype control antibody (labeled isotype control therapeutic) as described in FIG. 1. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 12 depicts photographs of lung sections from a murine model of asthma. Female BALB/c mice (8-10 wk of age) were sensitized with intraperitoneal (i.p.) injections of 20 μg ovalbumin (OVA) (0.1 ml of 200 μg/ml in PBS) every other day from day 1 through 19. Mice were challenged by nebulization with 10 mg/ml OVA in PBS for 40 minutes on days 33, 35, 37, and 39. One group of mice, labeled 10F4 prophylactic, was treated with 100 μg of monoclonal IgG1 hamster anti-mouse BLyS (clone 10F4) by i.p. injection on days 20 and 27, prior to challenge with OVA. Another group of mice, labeled isotype control prophylactic, was treated with an isotype control antibody (purified Armenian hamster IgG1 (clone G235-2356; BD Pharmingen) by i.p. injection on days 20 and 27, prior to challenge with OVA. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 13 is a graph illustrating the effects of prophylactic and therapeutic administration of anti-BLyS antibody on the amount of cellular lung infiltration in a murine model of asthma. Female BALB/c mice (8-10 wk of age) were sensitized with intraperitoneal (i.p.) injections of 20 μg ovalbumin (OVA) (0.1 ml of 200 μg/ml in PBS) every other day from day 1 through 19. Mice were challenged by nebulization with 10 mg/ml OVA in PBS for 40 minutes on days 33, 35, 37, and 39. One group of mice, labeled 10F4 prophylactic (Pro), was treated with 100 μg of monoclonal IgG1 hamster anti-mouse BLyS (clone 10F4) by i.p. injection on days 20 and 27, prior to challenge with OVA (prophylactic group). Another group of mice, labeled 10F4 therapeutic (Ther), was treated with monoclonal IgG1 hamster anti-mouse BLyS (clone 10F4) by i.p. injection on day 34 (therapeutic group). Controls included (1) a group of mice (Iso Pro) treated with 100 μg of an isotype control antibody (purified Armenian hamster IgG1 (clone G235-2356; BD Pharmingen)) by i.p. injection on days 20 and 27, prior to challenge with OVA (prophylactic group); (2) a group of mice (Iso Ther) treated with 100 μg of an isotype control antibody (purified Armenian hamster IgG1 (clone G235-2356; BD Pharmingen)) by i.p. injection on day 34 (therapeutic group); (3) a group of mice (Dex) treated with 50 μg of water-soluble dexamethasone in 100 μl of PBS by i.p. injection on days 34, 36, and 38 (i.e., every other day of the OVA challenge); (4) a group of mice (PBS) sensitized and challenged with PBS; and (5) a group of mice (Naïve) left untreated. All mice were terminated on day 40. FIG. 13 is a graph illustrating the percentage of lung area covered by cellular infiltrates (y-axis) for each treatment group (x-axis). The * represents a p value <0.05 and the ** represents a p<0.01.

FIGS. 14A-B are a series of graphs illustrating the effects of therapeutic administration of anti-BLyS antibody on the amount of eosinophil and mast cell lung infiltration in a murine model of asthma. OVA-sensitized and challenged mice were treated on day 34 with either 100 μg of monoclonal IgG1 hamster anti-mouse BLyS (clone 10F4) (labeled 10F4 Ther) or an isotype control antibody (labeled Isotype Ther) as described in FIG. 1. Another group of mice were sensitized and challenged with PBS (labeled PBS). FIG. 14A is a graph illustrating the number of eosinophils detected per mm² lung area (y-axis) for each treatment group (x-axis). FIG. 14B is a graph illustrating the number of mast cells detected per mm² lung area (y-axis) for each treatment group (x-axis).

FIGS. 15A-B are a series of graphs illustrating the effects of prophylactic and therapeutic administration of anti-BLyS antibody in a murine model of asthma. Female BALB/c mice (8-10 wk of age) were OVA-sensitized and treated as described in FIG. 1. FIG. 15A is a graph illustrating the percent of B220+ B cells in the spleen (y-axis) for each treatment group (x-axis). FIG. 15B is a graph illustrating the percent of B220+ B cells in the blood (y-axis) for each treatment group (x-axis).

FIGS. 16A-B are a series of graphs illustrating the effects of prophylactic and therapeutic administration of an anti-BLyS antibody and an anti-IL-13 antibody in a murine model of asthma. Female BALB/c mice (8-10 wk of age) were sensitized with intraperitoneal (i.p.) injections of 20 μg ovalbumin (OVA) (0.1 ml of 200 μg/ml in PBS) every other day from day 1 through 19. Mice were challenged by nebulization with 10 mg/ml OVA in PBS for 40 minutes on days 33, 35, 37, and 39. Prophylactic treatment groups (i.e., prior to challenge with OVA) included (1) mice treated with 100 μg of monoclonal IgG1 hamster anti-mouse BLyS (clone 10F4) by i.p. injection on days 20 and 27, labeled 10F4 prophylactic (Pro); (2) mice treated with an isotype control antibody (purified Armenian hamster IgG1 (clone G235-2356; BD Pharmingen)) by i.p. injection on days 20 and 27, labeled isotype (Iso) prophylactic (Pro); and (3) mice treated with 100 μg of anti-IL-13 antibody (R&D Systems; Cat. #MAB413) by i.p. injection on day 20 and 27, labeled anti-IL-13 prophylactic (Pro). Therapeutic treatment groups included (1) mice treated with monoclonal IgG1 hamster anti-mouse BLyS (clone 10F4) by i.p. injection on day 34, labeled 10F4 therapeutic (Ther); (2) mice treated with 100 μg of an isotype control antibody (purified Armenian hamster IgG1 (clone G235-2356; BD Pharmingen)) by i.p. injection on day 34, labeled isotype (Iso) therapeutic (Ther); (3) mice treated with 100 ug of anti-IL-13 antibody (R&D Systems; Cat. #MAB413) on day 34 by i.p. injection, labeled anti-IL-13 therapeutic (Ther); and (4) mice treated with 100 ug of anti-IgE antibody (BD Pharmingen; Cat. #553416) by i.p. injection on day 34, labeled anti-IgE therapeutic (Ther). In addition to the prophylactic and therapeutic isotype control groups, the following control groups also were included (1) a group of mice (Dex) treated with 50 μg of water-soluble dexamethasone in 100 μl of PBS by i.p. injection on days 34, 36, and 38 (i.e., every other day of the OVA challenge); (2) a group of mice (PBS) sensitized and challenged with PBS; and (3) a group of mice (Naïve) left untreated. All mice were terminated on day 40. FIG. 16A is a graph illustrating the total ng/ml of IgE detected in the serum (y-axis) for the isotype prophylactic, 10F4 prophylactic, anti-IL13 prophylactic, dexamethasone, PBS, and naïve treatment groups x-axis). FIG. 16B is a graph illustrating the total ng/ml of IgE detected in the serum (y-axis) for the isotype therapeutic, 10F4 therapeutic, anti-IL13 therapeutic, anti-IgE therapeutic, PBS, and naïve treatment groups (x-axis).

FIG. 17 is a graph illustrating the effects of prophylactic and therapeutic administration of an anti-BLyS antibody and an anti-IL-13 antibody in a murine model of asthma. Female BALB/c mice (8-10 wk of age) were OVA-sensitized and treated as described in FIG. 16. FIG. 17 is a graph illustrating the total ng/ml of BLyS protein detected in the serum (y-axis) for each treatment group (x-axis).

FIG. 18 is a graph illustrating the effects of prophylactic and therapeutic administration of an anti-BLyS antibody and an anti-IL-13 antibody in a murine model of asthma. Female BALB/c mice (8-10 wk of age) were OVA-sensitized and treated as described in FIG. 16. FIG. 18 is a graph illustrating the total pg/ml of IL-13 protein detected in the bronchoalveolar lavage fluid (y-axis) for each treatment group (x-axis).

FIGS. 19A-B are a series of graphs illustrating the effects of therapeutic administration of anti-BLyS antibody on the amount of eosinophil and neutrophil cell lung infiltration in a murine model of asthma. OVA-sensitized and challenged mice were treated on day 34 with either 100 μg of monoclonal IgG1 hamster anti-mouse BLyS (clone 10F4) (labeled 10F4 Ther) or an isotype control antibody (labeled Iso Ther) as described in FIG. 1. Another group of mice were sensitized and challenged with PBS (labeled PBS). FIG. 19A is a graph illustrating the number of eosinophils detected per mm² lung area (y-axis) for each treatment group (x-axis). FIG. 19B is a graph illustrating the number of neutrophils detected per mm² lung area (y-axis) for each treatment group (x-axis).

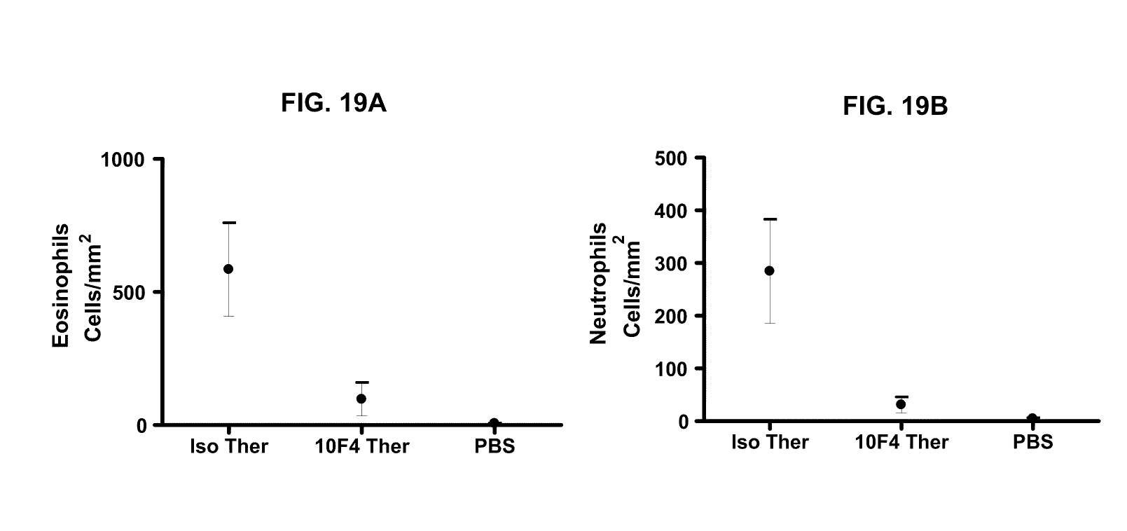

FIGS. 20A-C are a series of graphs illustrating the effects of administration of anti-BLyS antibody in a murine model of asthma. Female BALB/c mice (8-10 wk of age) were sensitized with intraperitoneal (i.p.) injections of 20 μg OVA (0.1 ml of 200 μg/ml in PBS) every other day from day 1 through 19. Mice were challenged by nebulization with 10 mg/ml OVA in PBS for 40 minutes on days 33, 35, 37, and 39. Mice were treated with either 100 μg of monoclonal IgG1 hamster anti-mouse BLyS (clone 10F4) or an isotype control antibody by i.p. injection on day 34, day 36, or day 38. All mice were terminated on day 40. FIG. 20A is a graph illustrating the total ng/ml of IgE detected in the serum (y-axis) for each treatment group (x-axis). FIG. 20B is a graph illustrating the percent of B220+ B cells in the spleen (y-axis) for each treatment group (x-axis). FIG. 20C is a graph illustrating the percent of B220+ B cells in the lung (y-axis) for each treatment group x-axis).

FIGS. 21A-B are a series of graphs illustrating the effects of prophylactic and therapeutic administration of an anti-BLyS antibody and an anti-IL-13 antibody in a murine model of asthma. Female BALB/c mice (8-10 wk of age) were treated as described in FIG. 16 and OVA-specific IgE levels were detected in serum isolated from each of the treatment groups. FIG. 21A is a graph illustrating the pg/ml of OVA-specific IgE detected in the serum (y-axis) for each prophylactic treatment group as compared to the dexamethasone (Dex), PBS, and naïve treatment groups x-axis). FIG. 21B is a graph illustrating the pg/ml of OVA-specific IgE detected in the serum (y-axis) for each therapeutic treatment group as compared to the dexamethasone (Dex), PBS, and naïve treatment groups x-axis).

FIG. 22 depicts photographs of lung sections from a murine model of asthma. OVA-sensitized and challenged mice were treated on day 34 with either 100 μg of an anti-IL-13 antibody (labeled anti-IL-13 therapeutic) or 100 μg of an anti-IgE antibody (labeled anti-IgE therapeutic) as described in FIG. 16. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 23 depicts photographs of lung sections from a murine model of asthma. OVA-sensitized and challenged mice were treated with either 100 ug of anti-IL-13 antibody on day 20 and 27 (labeled IL-13 prophylactic) or with 50 μg of water-soluble dexamethasone in 1000 of PBS on days 34, 36, and 38 (labeled Dexamethasone) as described in FIG. 16. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIGS. 24A-B are a series of graphs illustrating the effects of administration of anti-BLyS antibody in a murine model of chronic asthma. Female BALB/c mice (8-10 wk of age) were sensitized with intraperitoneal (i.p.) injections of 20 μg OVA (0.1 ml of 200 μg/ml in PBS) every other day from day 1 through 19. Mice were challenged by nebulization with 10 mg/ml OVA in PBS for 40 minutes on days 33, 35, 37, 39, 54, 56, 58, 60, 73, 75, 77, 79, 93, 95, 97, and 99. Mice were treated with either 100 μg of monoclonal IgG1 hamster anti-mouse BLyS (clone 10F4) or an isotype control antibody by i.p. injection on days 34 and 41 (d34/41), days 61 and 68 (d61/68), or days 80 and 87 (d80/87). In addition to the isotype control groups, the following control groups also were included (1) a group of mice sensitized and challenged with PBS (PBS); and (2) a group of mice sensitized and challenged with OVA and left untreated (No Rx). All mice were terminated on day 100. FIG. 24A is a graph illustrating the total ng/ml of IgE in the serum (y-axis) for each 10F4 treatment group, as well as the PBS and untreated control groups x-axis). FIG. 24B is a graph illustrating the total ng/ml of IgE in the BAL fluid (y-axis) for each 10F4 treatment group, as well as the untreated control group (x-axis).

FIG. 25 is a graph illustrating the effects of administration of anti-BLyS antibody in a murine model of chronic asthma. Female BALB/c mice (8-10 wk of age) were OVA-sensitized and treated as described in FIG. 24. FIG. 25 is a graph illustrating the percent of B220+ B cells in the lung (y-axis) for each treatment group, with the exception of the untreated group (x-axis).

FIGS. 26A-D are a series of graphs illustrating the effects of administration of anti-BLyS antibody in a murine model of chronic asthma. Female BALB/c mice (8-10 wk of age) were OVA-sensitized and treated as described in FIG. 24. FIG. 26A is a graph illustrating the total pg/ml of IL-13 protein detected in the bronchoalveolar lavage fluid (y-axis) for each 10F4 treatment group, as well as the untreated control group (x-axis). FIG. 26B is a graph illustrating the total pg/ml of IL-4 protein detected in the bronchoalveolar lavage fluid (y-axis) for each 10F4 treatment group, as well as the untreated and PBS control groups x-axis). FIG. 26C is a graph illustrating the total pg/ml of IL-5 protein detected in the bronchoalveolar lavage fluid (y-axis) for each 10F4 treatment group, as well as the untreated and PBS control groups x-axis). FIG. 26D is a graph illustrating the total pg/ml of TNF-α protein detected in the bronchoalveolar lavage fluid (y-axis) for each 10F4 treatment group, as well as the untreated and PBS control groups x-axis).

FIG. 27 depicts photographs of lung sections from a murine model of chronic asthma. OVA-sensitized and challenged mice were treated with either left untreated (labeled No Rx) or treated with 100 μg of monoclonal IgG1 hamster anti-mouse BLyS (clone 10F4) on days 34 and 41 (labeled d34/41 10F4Rx), on days 61 and 68 (labeled d61/68 10F4Rx), or on days 80 and 87 (labeled d80/87 10F4Rx) as described in FIG. 24. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 28 is a graph illustrating the effects of OVA sensitization and challenge on the circulating serum IgE levels over a period of time in a murine model of chronic asthma. Female BALB/c mice (8-10 wk of age) were sensitized with intraperitoneal (i.p.) injections of 20 μg OVA (0.1 ml of 200 μg/ml in PBS) every other day from day 1 through 19. Mice were challenged by nebulization with 10 mg/ml OVA in PBS for 40 minutes on days 33, 35, 37, 39, 47, 49, 51, 53, 61, 63, 65, 67, 120, 122, 124, 126, 152, 154, 156, 158, 249, 251, 253, and 255. Control mice were left untreated (naïve). Mice were terminated throughout the course of the experiment on days 40, 54, 68, 127, 159, and 256. FIG. 28 is a graph illustrating the total ng/ml of IgE in the serum (y-axis) for each treatment group (x-axis).

FIG. 29 is a graph illustrating the long-term effects of administration of anti-BLyS antibody in a murine model of chronic asthma. Female BALB/c mice (8-10 wk of age) were sensitized and challenged with OVA as described in FIG. 28. Mice were either treated with 100 μg of monoclonal IgG1 hamster anti-mouse BLyS (clone 10F4) by i.p. injection on days 34, 41, 48, and 55 (4× 10F4) or left untreated as a control (No Rx). Mice were terminated throughout the course of the experiment on days 54, 68, 127, and 256. FIG. 29 is a graph illustrating the total ng/ml of IgE in the serum (y-axis) for each treatment group (x-axis).

FIG. 30 is a graph illustrating the effects of administration of anti-BLyS antibody on the amount of cellular lung infiltration in a murine model of chronic asthma. Female BALB/c mice (8-10 wk of age) were sensitized and challenged with OVA as described in FIG. 28. Mice were treated with 100 μg of monoclonal IgG1 hamster anti-mouse BLyS (clone 10F4) by i.p. injection on days 34 and 41 (10F4 2×) or on days 34, 41, 48, and 55 (4× 10F4). Controls included (1) a group of mice sensitized and challenged with OVA and left untreated (UnRx) and (2) a group of mice left untreated (i.e., the mice were not sensitized, challenged, or treated) (naïve). Mice were terminated on day 68. FIG. 30 is a graph illustrating the percentage of lung area covered by cellular infiltrates (y-axis) for each treatment group (x-axis). The * represents a p value <0.05 as compared to the untreated mice (Unrx).

FIG. 31 depicts photographs of lung sections from a murine model of chronic asthma. Female BALB/c mice (8-10 wk of age) were OVA-sensitized and challenged as described in FIG. 28. Mice were either treated with 100 μg of monoclonal IgG1 hamster anti-mouse BLyS (clone 10F4) by i.p. injection on days 34, 41, 48, and 55 (4× 10F4) or left untreated as a control (No Rx). Mice were terminated on days 54 and 68. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 32 depicts photographs of lung sections from a murine model of chronic asthma. Female BALB/c mice (8-10 wk of age) were OVA-sensitized and challenged as described in FIG. 28. Mice were either treated with 100 μg of monoclonal IgG1 hamster anti-mouse BLyS (clone 10F4) by i.p. injection on days 34, 41, 48, and 55 (4× 10F4) or left untreated as a control (No Rx). Mice were terminated on days 127 and 159. Lung sections were isolated and stained with hematoxylin/eosin and alcian blue/periodic acid-Schiff (PAS) to detect mucus accumulation (red staining) and cellular infiltration (i.e., infiltration of cells of the immune system including, but not limited to, B cells, T cells, eosinophils, neutrophils and/or mast cells) (blue staining).

FIG. 33 is a graph illustrating the effects of administration of anti-BLyS antibody on B cell reconstitution in the spleen in a murine model of chronic asthma. Female BALB/c mice (8-10 wk of age) were OVA-sensitized and challenged as described in FIG. 28 and treated with 100 μg of monoclonal IgG1 hamster anti-mouse BLyS (clone 10F4) by i.p. injection on day 34 (10F4), on days 34 and 41 (2× 10F4), or on days 34, 41, 48, and 55 (4× 10F4). As a control, one group of mice was left untreated (i.e., the mice were not sensitized, challenged, or treated) (naïve). Mice were terminated throughout the course of the experiment on days 40, 68, 159, and 256. FIG. 33 is a graph illustrating the percentage of normalized B cells detected in the spleen (y-axis) for each treatment group (x-axis).

### DETAILED DESCRIPTION OF THE INVENTION

The invention relates to methods of using antagonists of B Lymphocyte Stimulator (BLyS) protein. In specific embodiments, the invention provides methods of using antagonists of B Lymphocyte Stimulator to treat, prevent, and/or ameliorate asthma, allergen-induced respiratory symptoms, or other allergic or inflammatory conditions of the respiratory system (e.g., the nostrils, nasopharynx, pharynx, trachea, bronchiole, bronchus, lungs, and/or alveoli) in a patient. B Lymphocyte Stimulator is also referred to in the art as Neutrokine-alpha, TALL-1, THANK, BAFF, zTNF4, or TNFSF13B. In a specific embodiment, the invention relates to the use of antibodies and related molecules that immunospecifically bind to B Lymphocyte Stimulator protein to treat, prevent, and/or ameliorate asthma, allergen-induced respiratory symptoms, or other allergic or inflammatory conditions of the respiratory system (e.g., the nostrils, nasopharynx, pharynx, trachea, bronchiole, bronchus, lungs, and/or alveoli) in a patient.

B Lymphocyte Stimulator protein is a member of the tumor necrosis factor (“TNF”) superfamily that induces both in vivo and in vitro B cell proliferation and differentiation (Moore et al., Science, 285: 260-263 (1999)). B Lymphocyte Stimulator protein shares amino acid sequence identity to a proliferation-inducing ligand (APRIL) (28.7%, SEQ ID NO:4), TNF-alpha (16.2%), and lymphotoxin-alpha (LT-alpha) (14.1%) (Moore, supra). The full length B Lymphocyte Stimulator gene encodes a 285 amino acid polypeptide that has a transmembrane spanning domain between amino acids 47 and 73 preceded by a non-hydrophobic sequence characteristic of type II membrane bound proteins. Like other members of the TNF family, B Lymphocyte Stimulator protein functions as a trimeric protein. Upon expression of B Lymphocyte Stimulator at the surface of the cell, the extracellular domain is cleaved off at amino acid 134 to release a biologically active trimer.

B Lymphocyte Stimulator protein is known to bind to three different receptors from the Tumor Necrosis Factor Receptor Superfamily. These receptors are transmembrane activator and CAML interactor (TACI, GenBank accession number AAC51790, SEQ ID NO:63), B-cell maturation antigen (BCMA, GenBank accession number NP 001183, SEQ ID NO:65), and B cell activating factor receptor (BAFF-R, GenBank accession number NP 443177, SEQ ID NO:67). (See, e.g., Gross, et al., (2000) Nature 404:995-999; Thompson et al., (2001) Science 293:2108-2111; and Yan et al., (2000) Nature Immunol. 1:252-256). Expression of the receptors is largely restricted to B lymphocytes (Moore, et al., (1999) Science 285:260-263).

B Lymphocyte Stimulator promotes B cell proliferation, differentiation, and survival. Circulating levels of B Lymphocyte Stimulator are an essential requirement for maturation of B cells (see, e.g., Treml et al., (2009) Cell Biochem. Biophys., 53(1): 1-16). Additionally, B Lymphocyte Stimulator has been shown to have some effect on T cells as well (see, e.g., MacKay et al., (1999) J. Exp. Med., 190:1697-1710; Huard et al., (2001) J. Immunol., 167:6225-6231; Huard et al., (2004) Int. Immunol., 16:467-475; Ng et al., (2004) J. Immunol., 173:807-817). As referred to herein, B Lymphocyte Stimulator protein encompasses full-length B Lymphocyte Stimulator protein, soluble B Lymphocyte Stimulator protein, membrane-bound B Lymphocyte Stimulator protein, fragments of B Lymphocyte Stimulator protein, derivatives of B Lymphocyte Stimulator protein, as well as splice variants of B Lymphocyte Stimulator protein. Methods of making, assaying, and using B Lymphocyte Stimulator protein and B Lymphocyte Stimulator antagonists have been described in U.S. Pat. No. 6,881,401 and U.S. Patent Application Publication 2009/0104189 A1, which are incorporated by reference herein.

Asthma is a disease of the lungs in which the airways become blocked or narrowed causing breathing difficulty. Asthma is commonly divided into two types: allergic (extrinsic) asthma and non-allergic (intrinsic) asthma.

Allergic (extrinsic) asthma is characterized by symptoms that are triggered by an allergic reaction. Allergic asthma is airway obstruction and inflammation that is partially reversible with medication. Allergic asthma is the most common form of asthma, affecting over 50% of people with asthma. Many of the symptoms of allergic and non-allergic asthma are the same (coughing, wheezing, shortness of breath or rapid breathing, and chest tightness). However, allergic asthma is triggered by inhaled allergens such as dust mite allergen, pet dander, pollen, mold, etc. resulting in asthma symptoms.

Non-Allergic (intrinsic) asthma is triggered by factors not related to allergies. Like allergic asthma, non-allergic asthma is characterized by airway obstruction and inflammation that is at least partially reversible with medication; however symptoms in this type of asthma are not associated with an allergic reaction. Although many of the symptoms of allergic and non-allergic asthma are the same, non-allergic asthma is triggered by other factors such as anxiety, stress, exercise, cold air, dry air, hyperventilation, smoke, viruses or other irritants. In non-allergic asthma, the immune system is not involved in the reaction.

Airway inflammation, airway obstruction, and airway hyper responsiveness are characteristic features of asthma. Airway hyper responsiveness is defined by an exaggerated response of the airways to nonspecific stimuli, which results in airway obstruction. A number of cytokines have been shown to be involved in airway inflammation, airway obstruction, and airway hyper responsiveness. See, e.g., Barnes, J Clin Invest., (2008) 118(11): 3546-3556. For example, IL-13 has been shown to induce many features of allergic lung disease, including airway hyper responsiveness, goblet cell metaplasia and mucus hypersecretion, which all contribute to airway obstruction. See, e.g., Wills-Karp et al., (1998) Science, 282(5397): 2258-2261.

The administration of B Lymphocyte Stimulator antagonists that inhibit an immune response, particularly the proliferation, differentiation, or survival of B-cells and/or T-cells, is an effective therapy in preventing and treating asthma or other allergic or inflammatory conditions of the nostrils, nasopharynx, pharynx, trachea, bronchiole, bronchus, lung, and/or alveoli. The administration of B Lymphocyte Stimulator antagonists also can be used to prevent and/or treat symptoms associated with asthma (e.g., symptoms associated with non-allergic or allergic asthma). The administration of B Lymphocyte Stimulator antagonists also can be used to prevent, ameliorate, and/or treat symptoms associated with other inflammatory conditions of the lung or respiratory system such as acute or chronic inflammation of the lung, pneumonia, emphysema, inflammatory lung injury, bronchiolitis obliterans, chronic bronchitis, pulmonary sarcoisosis, chronic obstructive pulmonary disease (COPD), interstitial lung disease, idiopathic pulmonary fibrosis, acute respiratory distress syndrome (ARDS), bronchiectasis, lung eosinophilia, interstitial fibrosis, and cystic fibrosis.

The administration of B Lymphocyte Stimulator antagonists also can be used to prevent, ameliorate, and/or treat symptoms associated with inflammatory conditions of the respiratory or nasal passages, such as chronic rhinosinusitis, since the same mechanisms underlie the inflammatory cascade in both the lung epithelium and the nasal epithelium. Chronic rhinosinusitis is characterized by inflammation of the mucosa of the nose and paranasal sinuses lasting more than 12 weeks. Clinical symptoms may include nasal blockage or congestion, nasal discharge, facial pain or pressure, reduction or loss of smell, endoscopic findings (polyps, mucopurulent discharge, and edema or obstruction), and/or CT scan abnormalities (mucosal changes within the ostiomeatal complex and/or sinuses). See, e.g., Fokkens et al., Allergy, 60: 583-601 (2005). Chronic rhinosinusitis often occurs concurrently with asthma or other inflammatory conditions of the lung.

The administration of B Lymphocyte Stimulator antagonists also can be used to prevent, ameliorate, and/or treat symptoms associated with an allergic condition of the lung or respiratory system, such as extrinsic allergic alveolitis (hypersensitivity pneumonitis) and eosinophilic lung disorders, including allergic bronchopulmonary aspergillosis, acute and chronic eosinophilic pneumonia, Churg-Strauss syndrome, and idiopathic hypereosinophilic syndrome.

For example, neutralization of B Lymphocyte Stimulator by administration of an antagonist can be used to treat asthma, including non-allergic and allergic asthma, to treat an allergic condition of the lung, to treat an allergic or inflammatory condition of the nostrils, nasopharynx, pharynx, trachea, bronchiole, bronchus, lung, and/or alveoli, to reduce total serum IgE or allergen-induced IgE in a patient suffering from asthma or an allergic or inflammatory condition of the nostrils, nasopharynx, pharynx, trachea, bronchiole, bronchus, lung and/or alveoli, to reduce cytokine levels in a patient suffering from asthma or an allergic or inflammatory condition of the nostrils, nasopharynx, pharynx, trachea, bronchiole, bronchus, lung, and/or alveoli, to prevent and/or treat allergen-induced airway hyper responsiveness, and to prevent and/or treat allergen-induced increase in mucus-containing cells in the airway epithelium.

In one embodiment, the invention provides a method of treating, preventing or ameliorating asthma (which includes non-allergic and allergic asthma) in a patient comprising administering to the patient an effective amount of a B Lymphocyte Stimulator antagonist, thereby treating, preventing or ameliorating asthma in the patient.

In another embodiment, the invention provides a method of treating, preventing or ameliorating an allergic condition of the lung or respiratory system, such as eosinophilic lung disorders, including allergic bronchopulmonary aspergillosis, acute and chronic eosinophilic pneumonia, Churg-Strauss syndrome, extrinsic allergic alveolitis (hypersensitivity pneumonitis), and idiopathic hypereosinophilic syndrome, in a patient comprising administering to the patient an effective amount of a B Lymphocyte Stimulator antagonist, thereby treating, preventing or ameliorating the allergic condition of the lung or respiratory system in the patient. The allergic condition of the lung or respiratory system can be characterized by an increase in allergen-induced airway hyper responsiveness and/or by an increase in mucus-containing cells.

In another embodiment, the invention provides a method of treating, preventing or ameliorating an inflammatory condition of the respiratory system (e.g., an inflammatory condition of the nostrils, nasopharynx, pharynx, trachea, bronchiole, bronchus, lungs, and/or alveoli) in a patient comprising administering to the patient an effective amount of a B Lymphocyte Stimulator antagonist, thereby treating, preventing or ameliorating respiratory system inflammation in the patient. The inflammatory condition of the respiratory system can be characterized by an increase in allergen-induced airway hyper responsiveness and/or by an increase in mucus-containing cells. In addition, the inflammatory condition of the respiratory system can be characterized by an increase in cellular infiltration of cells of the immune system such as B cells, T cells, eosinophils, neutrophils and/or mast cells or other leukocytes into the lung.

In one embodiment, the invention provides a method of treating, preventing or ameliorating chronic rhinosinusitis in a patient comprising administering to the patient an effective amount of a B Lymphocyte Stimulator antagonist, thereby treating, preventing or ameliorating chronic rhinosinusitis in the patient.

In another embodiment, the invention provides a method of treating, preventing or ameliorating an inflammatory condition of the lung in a patient comprising administering to the patient an effective amount of a B Lymphocyte Stimulator antagonist, thereby treating, preventing or ameliorating lung inflammation in the patient. The inflammatory condition of the lung can be characterized by an increase in allergen-induced airway hyper responsiveness and/or by an increase in mucus-containing cells. In addition, the inflammatory condition of the lung can be characterized by an increase in cellular infiltration of cells of the immune system such as B cells, T cells, eosinophils, neutrophils and/or mast cells or other leukocytes into the lung.

In another embodiment, the invention provides a method of reducing total serum IgE levels in a patient suffering from asthma or an allergic or inflammatory condition of the nostrils, nasopharynx, pharynx, trachea, bronchiole, bronchus, lung, and/or alveoli comprising administering to the patient an effective amount of a B Lymphocyte Stimulator antagonist, thereby reducing total serum IgE levels in the patient.

In another embodiment, the invention provides a method of reducing an allergen-induced increase in IgE levels in a patient suffering from asthma or an allergic or inflammatory condition of the nostrils, nasopharynx, pharynx, trachea, bronchiole, bronchus, lungs, and/or alveoli comprising administering to the patient an effective amount of a B Lymphocyte Stimulator antagonist, thereby reducing the allergen-induced increase in IgE levels in the patient.

In yet another embodiment, the invention provides a method of preventing allergen-induced airway hyper responsiveness in a patient comprising administering to the patient an effective amount of a B Lymphocyte Stimulator antagonist, thereby preventing allergen-induced airway hyper responsiveness in the patient. The invention also provides a method of treating allergen-induced airway hyper responsiveness in a patient comprising administering to the patient an effective amount of a B Lymphocyte Stimulator antagonist, thereby inhibiting allergen-induced airway hyper responsiveness in the patient.

In another embodiment, the invention provides a method of preventing an allergen-induced increase in mucus-containing cells or mucus accumulation or production in the airway epithelium of a patient comprising administering to the patient an effective amount of a B Lymphocyte Stimulator antagonist, thereby preventing an allergen-induced increase in mucus-containing cells or mucus accumulation or production in the airway epithelium of the patient. The invention also provides a method of treating allergen-induced increase in mucus-containing cells or mucus accumulation or production in the airway epithelium of a patient comprising administering to the patient an effective amount of a B Lymphocyte Stimulator antagonist, thereby inhibiting allergen-induced increase in mucus-containing cells or mucus accumulation or production in the airway epithelium of the patient. In another embodiment, the invention provides a method of preventing, treating and/or ameliorating an inflammatory condition of the lungs or respiratory system. Inflammatory conditions of the lungs or respiratory system include, but are not limited to, acute or chronic inflammation of the lung, pneumonia, emphysema, inflammatory lung injury, bronchiolitis obliterans, chronic bronchitis, pulmonary sarcoisosis, chronic obstructive pulmonary disease (COPD), interstitial lung disease, idiopathic pulmonary fibrosis, acute respiratory distress syndrome (ARDS), bronchiectasis, lung eosinophilia, interstitial fibrosis, and cystic fibrosis.

In another embodiment, the invention provides a method of reducing cytokine levels in a patient suffering from asthma or an allergic or inflammatory condition of the nostrils, nasopharynx, pharynx, trachea, bronchiole, bronchus, lung, and/or alveoli comprising administering to the patient an effective amount of a B Lymphocyte Stimulator antagonist, thereby reducing cytokine levels in the patient. In a further embodiment, the invention provides a method of treating, preventing or ameliorating an allergen-induced increase in cytokine production in the airway epithelium of a patient comprising administering to the patient an effective amount of an anti-B Lymphocyte Stimulator antagonist, thereby treating, preventing or ameliorating an allergen-induced increase in cytokine production in the airway epithelium of the patient. A number of cytokines, including lymphokines (cytokines that are secreted by T cells and regulate immune responses), proinflammatory cytokines (cytokines that amplify and perpetuate the inflammatory process), growth factors (cytokines that promote cell survival and result in structural changes in the airways), chemokines (cytokines that are chemotactic for inflammatory cells), and anti-inflammatory cytokines (cytokines that negatively modulate the inflammatory response) have been shown to be involved in airway inflammation, airway obstruction, and airway hyper responsiveness. See, e.g., Barnes, supra. Thus, in one embodiment, the invention provides a method of treating, preventing or ameliorating an allergen-induced increase in lymphokines, proinflammatory cytokines, growth factors, chemokines, and/or anti-inflammatory cytokines of a patient comprising administering to the patient an effective amount of an anti-B Lymphocyte Stimulator antagonist. Exemplary cytokines include, but are not limited to, IL-4, IL-5, IL-9, IL-12, IL-13, IL-17, IL-18, IL-25, IFN-γ, IL-1β, IL-6, TNF-α, TSLP, EGF, GM-CSF, NGF, SCF, TGF-β, VEGF, FGFs, TGF-α, CTGF, CCL2, CCL3, CCL4, CCL5, CCL9, CCL10, CCL11, CCL12, CCL13, CCL17, CCL22, CCL24, CCL26, CXCL1, CXCL5, CXCL8, CXCL9, CXCL10, CXCL11, and CXCL12.

In one embodiment, the invention provides a method of promoting tolerance to an allergen in a patient comprising administering to the patient an effective amount of a B Lymphocyte Stimulator antagonist before, during, or after exposure to the allergen, thereby ameliorating or preventing lung inflammation and promoting tolerance to the allergen in the patient.

In yet another embodiment, the invention provides the use of a B Lymphocyte Stimulator antagonist in the treatment, prevention, and/or amelioration of asthma, allergen-induced respiratory symptoms, or other allergic or inflammatory conditions of the lung or respiratory system. The invention also provides a B Lymphocyte Stimulator antagonist for use in the treatment, prevention, and/or amelioration of asthma, allergen-induced respiratory symptoms, or other allergic or inflammatory conditions of the lung or respiratory system. In addition, the invention provides the use of a B Lymphocyte Stimulator antagonist for the preparation of a medicament for the treatment, prevention, and/or amelioration of asthma, allergen-induced respiratory symptoms, or other allergic or inflammatory conditions of the lung or respiratory system.

In another embodiment, the invention provides the use of an anti-B Lymphocyte Stimulator antibody in the treatment, prevention, and/or amelioration of asthma, allergen-induced respiratory symptoms, or other allergic or inflammatory conditions of the lung or respiratory system. The invention also provides an anti-B Lymphocyte Stimulator antibody for use in the treatment, prevention, and/or amelioration of asthma, allergen-induced respiratory symptoms, or other allergic or inflammatory conditions of the lung or respiratory system. In addition, the invention provides the use of an anti-B Lymphocyte Stimulator antibody for the preparation of a medicament for the treatment, prevention, and/or amelioration of asthma, allergen-induced respiratory symptoms, or other allergic or inflammatory conditions of the lung or respiratory system.

B Lymphocyte Stimulator antagonists decrease or inhibit B Lymphocyte Stimulator-induced signal transduction. For example, antagonists of the invention may disrupt the interaction between B Lymphocyte Stimulator protein and its receptor to inhibit or downregulate B Lymphocyte Stimulator-induced signal transduction. Antagonists of the invention which do not prevent B Lymphocyte Stimulator from binding its receptor but inhibit or downregulate B Lymphocyte Stimulator-induced signal transduction also can be used in accordance with the invention set forth herein. In particular, antagonists of the invention which prevent B Lymphocyte Stimulator-induced signal transduction by specifically recognizing the unbound B Lymphocyte Stimulator protein, receptor-bound B Lymphocyte Stimulator protein, or both unbound and receptor-bound B Lymphocyte Stimulator protein can be used in accordance with the invention set forth herein.

In one embodiment, antagonists of the invention selectively target and inhibit the biological activity of soluble B Lymphocyte Stimulator (amino acids 134 to 285 of SEQ ID NO:2). In another embodiment, B Lymphocyte Stimulator antagonists of the invention induce autoreactive B-cell apoptosis when administered to a patient.

The ability of an antagonist of the invention to inhibit or downregulate B Lymphocyte Stimulator-induced signal transduction may be determined by techniques described herein or otherwise known in the art. For example, B Lymphocyte Stimulator-induced receptor activation and the activation of signaling molecules can be determined by detecting the phosphorylation (e.g., tyrosine or serine/threonine) of the receptor or a signaling molecule by immunoprecipitation followed by western blot analysis.

The B Lymphocyte Stimulator antagonist can be any B Lymphocyte Stimulator antagonist known to one of ordinary skill in the art, such as any B Lymphocyte Stimulator antagonist described herein including a protein comprising the B Lymphocyte Stimulator protein binding domain of TACI; a protein comprising the B Lymphocyte Stimulator protein domain of BCMA; a protein comprising the B Lymphocyte Stimulator binding domain of BAFF-R; a B Lymphocyte Stimulator binding peptide or polypeptide; a peptibody that binds B Lymphocyte Stimulator protein; a B Lymphocyte Stimulator protein variant; and/or an anti-B Lymphocyte Stimulator receptor antibody (e.g. TACI, BAFF-R, and/or BCMA).

Antagonists of B Lymphocyte Stimulator include binding and/or inhibitory antibodies, antisense nucleic acids, ribozymes, and inactive B Lymphocyte Stimulator polypeptides. These would be expected to find clinical or practical application, for example, as an immunosuppressive agent(s) or as an inhibitor of signaling pathways involving ERK1, COX2 and Cyclin D2 which have been associated with B Lymphocyte Stimulator induced B cell activation.

In one embodiment, the B Lymphocyte Stimulator antagonist is an anti-B Lymphocyte Stimulator antibody that binds to: (a) soluble B Lymphocyte Stimulator protein; (b) membrane-bound B Lymphocyte Stimulator protein, (c) the amino acid sequence of amino acid residues 1-285 of SEQ ID NO:2; (d) the amino acid sequence of amino acid residues 134-285 of SEQ ID NO:2; (e) a trimer of amino acid residues 134-285 of SEQ ID NO:2; (f) an amino acid sequence that is at least 90% identical to amino acid residues 1-285 of SEQ ID NO:2, wherein the amino acid sequence stimulates B cell proliferation, differentiation, or survival; (g) an amino acid sequence that is at least 90% identical to amino acid residues 134-285 of SEQ ID NO:2, wherein the amino acid sequence stimulates B cell proliferation, differentiation, or survival; (h) a trimer of an amino acid sequence that is at least 90% (e.g., at least 91%, at least 92%, at least 93%, at least 94%, at least 95%, at least 96%, at least 97%, at least 98%, at least 99%, or 100% identical) identical to amino acid residues 134-285 of SEQ ID NO:2; and (i) the amino acid sequence of a fragment of the polypeptide of SEQ ID NO:2; wherein the fragment is at least 30 amino acids in length and wherein the fragment is capable of stimulating B cell proliferation, differentiation, or survival.

In a preferred embodiment, the anti-B Lymphocyte Stimulator antibody comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to the VH and/or VL domains of SEQ ID NO: 60 or SEQ ID NO: 61. In another preferred embodiment, the anti-B Lymphocyte Stimulator antibody is BENLYSTA® (belimumab) from Human Genome Sciences, Inc. In another preferred embodiment, the anti-B Lymphocyte Stimulator antibody comprises an amino acid sequence that is at least 80%, at least 85%, at least 90%, at least 91%, at least 92%, at least 93%, at least 94%, at least 95%, at least 96%, at least 97%, at least 98%, at least 99%, or 100% identical to residues 1-126 of SEQ ID NO:72 and an amino acid sequence that is at least 85% identical to residues 143-251 of SEQ ID NO:73. In another preferred embodiment, the anti-B Lymphocyte Stimulator antibody comprises a first amino acid sequence that is at least 80%, at least 85%, at least 90%, at least 91%, at least 92%, at least 93%, at least 94%, at least 95%, at least 96%, at least 97%, at least 98%, at least 99%, or 100% identical to residues 1-126 of SEQ ID NO:72 and a second amino acid sequence that is at least 85% identical to residues 143-251 of SEQ ID NO:73. In another embodiment, the anti-B Lymphocyte Stimulator antibody is the antibody disclosed and claimed in U.S. Pat. No. 7,605,236, which is incorporated by reference herein.

In another embodiment, the anti-B Lymphocyte Stimulator antibody is LY2127399 (Eli Lilly and Co., Indianapolis, Ind.), which is a fully human IgG4 monoclonal antibody with neutralizing activity against both membrane-bound and soluble B Lymphocyte Stimulator. See Kikly et al., Characterization of LY2127399, A Neutralizing Antibody for BAFF (Arthritis & Rheumatism, Volume 60, October 2009 Abstract Supplement). In another embodiment, the anti-B Lymphocyte Stimulator antibody is the antibody disclosed and claimed in U.S. Pat. No. 7,317,089, which is incorporated by reference herein.

In one embodiment, the B Lymphocyte Stimulator antagonist comprises a B Lymphocyte Stimulator receptor (e.g. TACI, BAFF-R, and/or BCMA) or fragment thereof fused to a heterologous protein such as an Fc domain of an immunoglobulin (e.g., IgG, IgA, IgE, IgM, or IgD). For example, the B Lymphocyte Stimulator antagonist can comprise TACI fused to an immunoglobulin G1 Fc domain. In a specific embodiment, the B Lymphocyte Stimulator antagonist is ATACICEPT™ (CAS Registry Number 845264-92-8).

B Lymphocyte Stimulator receptor proteins, fragments and variants thereof, as well as antibodies there to have been described in, for example, PCT Publications WO03/014294, WO02/066516, WO02/024909 WO03/014294, WO03/024991, WO02/094852 and WO04/011611 and U.S. Patent Publication Nos. US20030148445, US20030099990, US2005070689, US2005043516 and US2003012783. Each of the aforementioned references is herein incorporated by reference in its entirety.

In preferred embodiments the B Lymphocyte Stimulator receptors are soluble. In other preferred embodiments the B Lymphocyte Stimulator receptors are fused to the Fc region of an immunoglobulin molecule (e.g., amino acid residues 1-154 of TACI (GenBank accession number AAC51790, SEQ ID NO:63), amino acids 1-48 of BCMA (GenBank accession number NP 001183, SEQ ID NO:65) or amino acids 1-81 of BAFF-R (GenBank accession number NP 443177, SEQ ID NO:67) fused to the Fc region of an IgG molecule).

In another embodiment, a BAFF-R protein that may be used in the methods of the present invention is an isolated polypeptide comprising amino acids 1 to 70 of SEQ ID NO:67 and/or the amino acid sequence of SEQ ID NO:71. SEQ ID NO:71 shows amino acids 1-70 of BAFF-R wherein amino acid 20 (valine) in BAFF-R is substituted with asparagine and amino acid 27 (leucine) in BAFF-R is substituted with proline. In another embodiment, a BAFF-R protein that may be used in the methods of the present invention is an isolated polypeptide comprising amino acids 2 to 70 of SEQ ID NO:71. Polypeptides which are at least 80% identical, more preferably at least 90% or 95% identical, still more preferably at least 96%, 97%, 98%, 99% or 100% identical to the polypeptides described above may also be used in the methods of the present invention.

In a one embodiment, the B Lymphocyte Stimulator antagonist is a B Lymphocyte Stimulator binding peptide or polypeptide. B Lymphocyte Stimulator binding peptides or polypeptides have been described in, for example International Patent Publication numbers WO05/005462, WO05/000351, WO02/092620, WO02/16412, WO02/02641 and WO02/16411 and US Patent Publication numbers US2006135430, US2006084608, US2003194743, US20030195156 and US2003091565, each of which is herein incorporated by reference in its entirety. B Lymphocyte Stimulator binding peptides or polypeptides also have been described in, for example Sun et al., (2006) Biochem. Biophys. Res. Commun. 346:1158-1162 which is herein incorporated by reference in its entirety. B Lymphocyte Stimulator binding peptides that may be used in the methods of the present invention include short polypeptides identified from random peptide sequences displayed by fusion with coat proteins of filamentous phage. For discussion of phage display peptide library technology see, for example, Scott et al. (1990), Science 249: 386; Devlin et al. (1990), Science 249: 404; U.S. Pat. No. 5,223,409, issued Jun. 29, 1993; U.S. Pat. No. 5,733,731, issued Mar. 31, 1998; U.S. Pat. No. 5,498,530, issued Mar. 12, 1996; U.S. Pat. No. 5,432,018, issued Jul. 11, 1995; U.S. Pat. No. 5,338,665, issued Aug. 16, 1994; U.S. Pat. No. 5,922,545, issued Jul. 13, 1999; WO 96/40987, published Dec. 19, 1996; and WO 98/15833, published Apr. 16, 1998 (each of which is incorporated by reference in its entirety).

In preferred embodiments, the B Lymphocyte Stimulator binding peptides that may be used in the methods of the invention include the amino acid sequence of SEQ ID NO:68, the amino acid sequence of SEQ ID NO:69 or the amino acid sequence of SEQ ID NO:70.

In one embodiment, the B Lymphocyte Stimulator antagonist comprises a B Lymphocyte Stimulator peptibody. Exemplary B Lymphocyte Stimulator peptibodies that may be used in the invention are described in U.S. Pat. No. 7,259,137, which is incorporated herein by reference. In a specific embodiment, the B Lymphocyte Stimulator antagonist is AGP3 peptibody, which is described in U.S. Pat. No. 7,259,137. In another embodiment, the B Lymphocyte Stimulator antagonist is A-623 peptibody from Anthera Pharmaceuticals. In another embodiment, the B Lymphocyte Stimulator antagonist is a peptibody comprising an amino acid sequence that is at least 85%, at least 90%, at least 91%, at least 92%, at least 93%, at least 94%, at least 95%, at least 96%, at least 97%, at least 98%, at least 99%, or 100% identical to SEQ ID NO:68. In another embodiment, the B Lymphocyte Stimulator antagonist is a peptibody consisting of SEQ ID NO:68.

B Lymphocyte Stimulator antagonists can be administered before, during, and/or after the onset of asthma symptoms or symptoms of an allergic or inflammatory condition of the lung or respiratory system. In one embodiment, the B Lymphocyte Stimulator antagonist is initially administered before the onset of symptoms of asthma (which includesallergic and non-allergic asthma) or an allergic or inflammatory condition of the lung or respiratory system. For example, B Lymphocyte Stimulator antagonists can be administered to a patient who has been diagnosed with asthma or an allergic or inflammatory condition of the lung or respiratory system but who is not experiencing any symptoms in order to prevent the recurrence of asthma symptoms or symptoms of an allergic or inflammatory condition of the lung or respiratory system in the patient. Alternatively, B Lymphocyte Stimulator antagonists can be administered to a patient who has been diagnosed with asthma or an allergic or inflammatory condition of the lung or respiratory system and who is experiencing one or more symptoms (e.g., nasal blockage, wheezing, coughing, chest tightness, airflow obstruction, bronchospasm, bronchoconstriction or shortness of breath) in order to treat or ameliorate the one or more asthma symptoms or symptoms of an allergic or inflammatory condition of the lung or respiratory system in the patient.

In one embodiment of the invention, a patient is treated with an initial dose of a B Lymphocyte Stimulator antagonist followed by periodic maintenance doses of the B Lymphocyte Stimulator antagonist. Such maintenance does can be administered as short as one year or as long as the life of the patient in order to treat and/or prevent asthma or an allergic or inflammatory condition of the lung. For example, maintenance doses of the B Lymphocyte Stimulator antagonist can be administered for 1, 2, 3, 4, 5, 6, 7, 8, 9, 10, 15, or years. Maintenance doses of the B Lymphocyte Stimulator antagonist can be administered, for example, about once per week, about once per every two weeks, about once per month, or as regularly administered by one skilled in the art. In one embodiment, the maintenance dose is administered about once per every two, three, four, five, six, seven, eight, nine, ten, eleven, or twelve months.

In some instances maintenance doses of the B Lymphocyte Stimulator antagonist are not required for the life of the patient. Thus, in one embodiment of the invention, maintenance doses of the B Lymphocyte Stimulator antagonist can be reduced, tapered off, and/or eventually discontinued, such that the B Lymphocyte Stimulator antagonist is administered for only about three months, six months, one year, eighteen months, or two years. In some instances, the B Lymphocyte Stimulator antagonist may be administered for only about one, two, three, four, five, six, seven, eight, nine, ten, eleven, or twelve weeks.

Effective reduction or tapering of the B Lymphocyte Stimulator maintenance dose may be accomplished either by reducing the frequency of administration of the maintenance dose or by reducing the concentration of the maintenance dose over time. For example, the concentration of the maintenance dose may be reduced by administering about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% less B Lymphocyte Stimulator antagonist per each maintenance dose. For example, the maintenance dose may be reduced by about 10% per each administration until the concentration of the maintenance dose reaches zero. Alternatively, the maintenance dose may be tapered by reducing the maintenance dose by 10%, then by 20%, then by 40%, etc., until the maintenance dose reaches zero. Additional tapering regimens can be determined by one of ordinary skill in the art based on the patient's response to the B Lymphocyte Stimulator antagonist tapering.

In some embodiments, the B Lymphocyte Stimulator antagonist is administered following a diagnosis of increased IgE antibody titer followed by doses of the B Lymphocyte Stimulator antagonist until IgE antibody titer decreases.

Preferably, treatment using B Lymphocyte Stimulator antagonists is accomplished by administering an effective amount of a B Lymphocyte Stimulator antagonist to the patient.

### Formulations and Administration

The B Lymphocyte Stimulator antagonists will be formulated and dosed in a fashion consistent with good medical practice, taking into account the clinical condition of the individual patient (especially the side effects of treatment with B Lymphocyte Stimulator antagonists alone), the site of delivery of the B Lymphocyte Stimulator antagonist, the method of administration, the scheduling of administration, and other factors known to practitioners.

The “effective amount” of B Lymphocyte Stimulator antagonist(s) for purposes herein is thus determined by such considerations. In particular, effective dosages of the B Lymphocyte Stimulator antagonist(s) to be administered may be determined through procedures well known to those in the art which address such parameters as biological half-life, bioavailability, and toxicity. Such determination is well within the capability of those skilled in the art, especially in light of the detailed disclosure provided herein.

As a general proposition, the total pharmaceutically effective amount of a B Lymphocyte Stimulator antagonist administered parenterally per dose will be in the range of about 1 microgram/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day.

In another embodiment, the B Lymphocyte Stimulator antagonist is administered to a human at a dose between 0.0001 and 0.045 mg/kg/day, preferably at a dose between 0.0045 and 0.045 mg/kg/day, and more preferably at a dose of about 45 microgram/kg/day in humans, and at a dose of about 3 mg/kg/day in mice.

If given continuously, the B Lymphocyte Stimulator antagonist is typically administered at a dose rate of about 1 microgram/kg/hour to about 50 micrograms/kg/hour, either by 1-4 injections per day or by continuous subcutaneous infusions, for example, using a mini-pump. An intravenous bag solution may also be employed.

The length of treatment needed to observe changes and the interval following treatment for responses to occur appears to vary depending on the desired effect.

In a specific embodiment, the total pharmaceutically effective amount of B Lymphocyte Stimulator antagonist administered parenterally per dose will be in the range of about 0.1 microgram/kg/day to 45 micrograms/kg/day of patient body weight, although, as noted above, this will be subject to therapeutic discretion. More preferably, this dose is at least 0.1 microgram/kg/day, and most preferably for humans between about 0.01 and 50 micrograms/kg/day for the protein. B Lymphocyte Stimulator antagonists may be administered as a continuous infusion, multiple discrete injections per day (e.g., three or more times daily, or twice daily), single injection per day, or as discrete injections given intermittently (e.g., twice daily, once daily, every other day, twice weekly, weekly, biweekly, monthly, bimonthly, and quarterly). If given continuously, the B Lymphocyte Stimulator antagonist is typically administered at a dose rate of about 0.001 to 10 microgram/kg/hour to about 50 micrograms/kg/hour, either by 1-4 injections per day or by continuous subcutaneous infusions, for example, using a mini-pump.

Bioexposure of an organism to B Lymphocyte Stimulator antagonists during therapy may also play an important role in determining a therapeutically and/or pharmacologically effective dosing regime. Variations of dosing such as repeated administrations of a relatively low dose of B Lymphocyte Stimulator antagonists for a relatively long period of time may have an effect which is therapeutically and/or pharmacologically distinguishable from that achieved with repeated administrations of a relatively high dose of B Lymphocyte Stimulator antagonists for a relatively short period of time.

Using the equivalent surface area dosage conversion factors supplied by Freireich, E. J., et al. (Cancer Chemotherapy Reports 50(4):219-44 (1966)), one of ordinary skill in the art is able to conveniently convert data obtained from the use of B Lymphocyte Stimulator antagonists in a given experimental system into an accurate estimation of a pharmaceutically effective amount of B Lymphocyte Stimulator antagonists to be administered per dose in another experimental system. Experimental data obtained through the administration of B Lymphocyte Stimulator antagonists in mice may be converted through the conversion factors supplied by Freireich, et al., to accurate estimates of pharmaceutically effective doses of B Lymphocyte Stimulator antagonists in rat, monkey, dog, and human. The following conversion table (Table 1) is a summary of the data provided by Freireich, et al. Table 1 gives approximate factors for converting doses expressed in terms of mg/kg from one species to an equivalent surface area dose expressed as mg/kg in another species tabulated.

TABLE 1
Equivalent Surface Area Dosage Conversion Factors.

| | TO | | | | |
|---|---|---|---|---|---|
| | Mouse | Rat | Monkey | Dog | Human |
| FROM | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | 5/3 | 1 | ½ |
| Human | 12 | 7 | 3 | 2 | 1 |

Thus, for example, using the conversion factors provided in Table 1, a dose of 50 mg/kg in the mouse converts to an appropriate dose of 12.5 mg/kg in the monkey because (50 mg/kg)×(¼)=12.5 mg/kg. As an additional example, doses of 0.02, 0.08, 0.8, 2, and 8 mg/kg in the mouse equate to effect doses of 1.667 micrograms/kg, 6.67 micrograms/kg, 66.7 micrograms/kg, 166.7 micrograms/kg, and 0.667 mg/kg, respectively, in the human.

In certain embodiments, administration of radiolabeled forms of B Lymphocyte Stimulator antagonists (e.g., antibodies) is contemplated. The radiometric dosage to be applied can vary substantially. The radiolabeled B Lymphocyte Stimulator antagonist composition can be administered at a dose of about 0.1 to about 100 mCi per 70 kg body weight. In another embodiment, the radiolabeled B Lymphocyte Stimulator antagonist composition can be administered at a dose of about 0.1 to about 50 mCi per 70 kg body weight. In another embodiment, the radiolabeled B Lymphocyte Stimulator antagonist composition can be administered at a dose of about 0.1, 0.5, 1, 5, 10, 15, 20, 25, 30, 35, 40, 50, 60, 70, 80, 90 or 100 mCi per 70 kg body weight.

The radiolabeled B Lymphocyte Stimulator antagonist composition can be administered at a dose of about 0.1 to about 10 mCi/kg body weight. In another embodiment, the radiolabeled B Lymphocyte Stimulator antibody antagonist can be administered at a dose of about 0.25 to about 5 mCi/kg body weight. In specific embodiments, the radiolabeled B Lymphocyte Stimulator antagonist composition can be administered at a dose of about 0.35, 0.70, 1.35, 1.70, 2.0, 2.5 or 3.0 mCi/kg.

The radiolabeled B Lymphocyte Stimulator antagonist composition can be administered at a dose of about 1 to about 50 mCi/m². In another embodiment, the radiolabeled B Lymphocyte Stimulator antagonist composition can be administered at a dose of about 10 to about 30 mCi/m². In specific embodiments, the radiolabeled B Lymphocyte Stimulator antagonist composition can be administered at a dose of about 10, 15, 20, 25, or mCi/m².

The concentration of total B Lymphocyte Stimulator antagonist in a radiolabeled B Lymphocyte Stimulator antagonist composition may also vary, for example from about 1 microgram/kg to about 1 mg/kg. In specific embodiments, the total concentration of B Lymphocyte Stimulator antagonist in a radiolabeled B Lymphocyte Stimulator antagonist composition may be about 5, 10, 15, 20, 25, 30, 35, 40, 45, 50, 55, 60, 65, 70, 75, 80, 85, 90, 95, or 100 micrograms/kg.

An amount of radioactivity which would provide approximately 500 cGy to the whole body of a human is estimated to be about 825 mCi of ¹³¹I. The amounts of radioactivity to be administered depend, in part, upon the isotope chosen. For ⁹⁰Y therapy, from about 1 to about 200 mCi amounts of radioactivity are considered appropriate, with preferable amounts being 1 to 150 mCi, and 1 to 100 mCi (e.g., 60 mCi) being most preferred. The preferred means of estimating tissue doses from the amount of administered radioactivity is to perform an imaging or other pharmacokinetic regimen with a tracer dose, so as to obtain estimates of predicted dosimetry. In determining the appropriate dosage of radiopharmaceutical to administer to an individual, it is necessary to consider the amount of radiation that individual organs will receive compared to the maximum tolerance for such organs. Such information is known to those skilled in the art, for example, see Emami et al., International Journal of Radiation Oncology, Biology, Physics 21:109-22 (1991); and Meredith, Cancer Biotherapy & Radiopharmaceuticals 17:83-99 (2002), both of which are hereby incorporated by reference in their entireties.

A “high-dose” protocol, for example in the range of 200 to 600 cGy (or higher) to the whole body, may require the support of a bone-marrow replacement protocol, as the bone-marrow is the tissue which limits the radiation dosage due to toxicity.

In one embodiment, compositions comprising iodinated forms of the B Lymphocyte Stimulator antagonist (e.g., antibody) may also comprise radioprotectants and plasma expanders such as sodium ascorbate, gentran-40, and glycerol. In specific embodiments, compositions comprising iodinated forms of B Lymphocyte Stimulator antagonists are formulated in 10.0 mM sodium citrate, 140.0 mM sodium chloride, 8.7 mM HEPES, 4% (w/v) sodium ascorbate, 3.3% (w/v) Genetran-40.

The B Lymphocyte Stimulator antagonist may be administered alone or in a composition (e.g., a pharmaceutical composition) comprising a carrier, such as a pharmaceutically acceptable carrier. In one embodiment, “pharmaceutically acceptable carrier” means a non-toxic solid, semisolid or liquid filler, diluent, encapsulating material or formulation auxiliary of any type. In a specific embodiment, “pharmaceutically acceptable” means approved by a regulatory agency of the federal or a state government or listed in the U.S. Pharmacopeia or other generally recognized pharmacopeia for use in animals, and more particularly humans. Nonlimiting examples of suitable pharmaceutical carriers according to this embodiment are provided in “Remington's Pharmaceutical Sciences” by E. W. Martin, and include sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The B Lymphocyte Stimulator antagonist may be administered alone or in combination with other therapeutic agents, including but not limited to, chemotherapeutic agents, antibiotics, antivirals, steroidal and non-steroidal anti-inflammatories, conventional immunotherapeutic agents and cytokines Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The B Lymphocyte Stimulator antagonist may be administered alone or in combination with one or more adjuvants. Adjuvants that may be administered with the B Lymphocyte Stimulator antagonist include, but are not limited to, alum, alum plus deoxycholate (ImmunoAg), MTP-PE (Biocine Corp.), QS21 (Genentech, Inc.), BCG, and MPL. In a specific embodiment, B Lymphocyte Stimulator antagonists are administered in combination with alum. In another specific embodiment, B Lymphocyte Stimulator antagonists are administered in combination with QS-21. Further adjuvants that may be administered with the B Lymphocyte Stimulator antagonists include, but are not limited to, monophosphoryl lipid immunomodulator, AdjuVax 100a, QS-21, QS-18, CRL1005, aluminum salts, MF-59, and virosomal adjuvant technology.

In a further embodiment, the B Lymphocyte Stimulator antagonists are administered in combination with an antibiotic agent. Antibiotic agents that may be administered include, but are not limited to, amoxicillin, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamethoxazole, and vancomycin.

Conventional nonspecific immunosuppressive agents that may be administered in combination with the B Lymphocyte Stimulator antagonists include, but are not limited to, steroids, cyclosporine, cyclosporine analogs, cyclophosphamide, cyclophosphamide IV, methylprednisolone, prednisolone, azathioprine, FK-506, 15-deoxyspergualin, and other immunosuppressive agents that act by suppressing the function of responding T cells. Other immunosuppressive agents, that may be administered in combination with the B Lymphocyte Stimulator antagonists include, but are not limited to, prednisolone, methotrexate, thalidomide, methoxsalen, rapamycin, leflunomide, mizoribine (BREDININ™), brequinar, deoxyspergualin, and azaspirane (SKF 105685).

In specific embodiments, B Lymphocyte Stimulator antagonists are administered in combination with immunosuppressants. Immunosuppressant preparations that may be administered include, but are not limited to, ORTHOCLONE OKT® 3 (muromonab-CD3), SANDIMMUNET™, NEORAL™, SANGDYA™ (cyclosporine), PROGRAF® (FK506, tacrolimus), CELLCEPT® (mycophenolate motefil, of which the active metabolite is mycophenolic acid), IMURAN™ (azathioprine), glucorticosteroids, adrenocortical steroids such as DELTASONE™ (prednisone) and HYDELTRASOL™ (prednisolone), FOLEX™ and MEXATE™ (methotrexate), OXSORALEN-ULTRA™ (methoxsalen), RITUXAN™ (rituximab), and RAPAMUNET™ (sirolimus).

In a preferred embodiment, the B Lymphocyte Stimulator antagonists are administered in combination with steroid therapy. Steroids that may be administered include, but are not limited to, oral corticosteroids, prednisone, and methylprednisolone (e.g., IV methylprednisolone). In a specific embodiment, the B Lymphocyte Stimulator antagonists are administered in combination with prednisone. In a further specific embodiment, the B Lymphocyte Stimulator antagonists are administered in combination with prednisone and an immunosuppressive agent. Immunosuppressive agents that may be administered with prednisone are those described herein, and include, but are not limited to, azathioprine, cyclophosphamide, and cyclophosphamide IV. In another specific embodiment, the B Lymphocyte Stimulator antagonists are administered in combination with methylprednisolone. In a further specific embodiment, the B Lymphocyte Stimulator antagonists are administered in combination with methylprednisolone and an immunosuppressive agent. Immunosuppressive agents that may be administered with methylprednisolone are those described herein, and include, but are not limited to, azathioprine, cyclophosphamide, and cyclophosphamide IV.

In a preferred embodiment, the B Lymphocyte Stimulator antagonists are administered in combination with an antimalarial. Antimalarials that may be administered include, but are not limited to, hydroxychloroquine, chloroquine, and/or quinacrine.

In a preferred embodiment, B Lymphocyte Stimulator antagonists are administered in combination with an NSAID.

In an additional embodiment, B Lymphocyte Stimulator antagonists are administered alone or in combination with one or more intravenous immune globulin preparations. Intravenous immune globulin preparations that may be administered include, but not limited to, GAMMAR™, IVEEGAM™, SANDOGLOBULIN™, GAMMAGARD S/D™, and GAMIMUNET™.

In an additional embodiment, B Lymphocyte Stimulator antagonists are administered alone or in combination with an anti-inflammatory agent. Anti-inflammatory agents that may be administered include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

Pharmaceutical compositions containing B Lymphocyte Stimulator antagonists may be administered orally, rectally, parenterally, subcutaneously, intracisternally, intravaginally, intraperitoneally, topically (as by powders, ointments, drops or transdermal patch), bucally, or as an oral or nasal spray (e.g., via inhalation of a vapor or powder). The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of a B Lymphocyte Stimulator antagonist, preferably in purified form, together with a suitable amount of carrier so as to provide the form for proper administration to the patient. The formulation should suit the mode of administration.

The term “parenteral” as used herein refers to modes of administration which include intravenous, intramuscular, intraperitoneal, intrasternal, subcutaneous and intraarticular injection and infusion.

In a preferred embodiment, B Lymphocyte Stimulator antagonists are administered subcutaneously. In another preferred embodiment, B Lymphocyte Stimulator antagonists are administered through the nasal cavity or nasal mucosa. Pharmaceutical compositions for intranasal administration are well known in the art.

In another preferred embodiment, B Lymphocyte Stimulator antagonists are administered intravenously as a pharmaceutical composition. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

B Lymphocyte Stimulator antagonists are also suitably administered by sustained-release systems. Suitable examples of sustained-release compositions include suitable polymeric materials (such as, for example, semi-permeable polymer matrices in the form of shaped articles, e.g., films, or microcapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, and EP 0058481), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 0133988).

In a preferred embodiment, B Lymphocyte Stimulator antagonists are formulated in a biodegradable, polymeric drug delivery system, for example as described in U.S. Pat. Nos. 4,938,763; 5,278,201; 5,278,202; 5,324,519; 5,340,849; and 5,487,897 and in International Patent Application Publications WO 01/35929, WO 00/24374, and WO 00/06117 which are hereby incorporated by reference in their entirety. In specific preferred embodiments the B Lymphocyte Stimulator antagonists are formulated using the ATRIGEL® Biodegradable System of Atrix Laboratories, Inc. (Fort Collins, Colo.). In other specific embodiments, B Lymphocyte Stimulator antagonists are formulated using the ProLease® sustained release system available from Alkermes, Inc. (Cambridge, Mass.).

Examples of biodegradable polymers which can be used in the formulation of B Lymphocyte Stimulator antagonists, include but are not limited to, polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), poly(methyl vinyl ether), poly(maleic anhydride), polyvinylpyrrolidone, polyethylene glycol, polyhydroxycellulose, chitin, chitosan, and copolymers, terpolymers, or combinations or mixtures of the above materials. The preferred polymers are those that have a lower degree of crystallization and are more hydrophobic. These polymers and copolymers are more soluble in the biocompatible solvents than the highly crystalline polymers such as polyglycolide and chitin which also have a high degree of hydrogen-bonding. Preferred materials with the desired solubility parameters are the polylactides, polycaprolactones, and copolymers of these with glycolide in which there are more amorphous regions to enhance solubility. In specific preferred embodiments, the biodegradable polymers which can be used in the formulation of B Lymphocyte Stimulator antagonists are poly(lactide-co-glycolides). Polymer properties such as molecular weight, hydrophobicity, and lactide/glycolide ratio may be modified to obtain the desired drug B Lymphocyte Stimulator antagonist release profile (See, e.g., Ravivarapu et al., Journal of Pharmaceutical Sciences 89:732-741 (2000), which is hereby incorporated by reference in its entirety).

It is also preferred that the solvent for the biodegradable polymer be non-toxic, water miscible, and otherwise biocompatible. Examples of such solvents include, but are not limited to, N-methyl-2-pyrrolidone, 2-pyrrolidone, C2 to C6 alkanols, C1 to C15 alcohols, diols, triols, and tetraols such as ethanol, glycerine propylene glycol, and butanol; C3 to C15 alkyl ketones such as acetone, diethyl ketone, and methyl ethyl ketone; C3 to C15 esters such as methyl acetate, ethyl acetate, and ethyl lactate; alkyl ketones such as methyl ethyl ketone; C1 to C15 amides such as dimethylformamide, dimethylacetamide, and caprolactam; C3 to C20 ethers such as tetrahydrofuran and solketal; tweens, triacetin, propylene carbonate, decylmethylsulfoxide, dimethyl sulfoxide, oleic acid, 1-dodecylazacycloheptan-2-one. Other preferred solvents are benzyl alchohol, benzyl benzoate, dipropylene glycol, tributyrin, ethyl oleate, glycerin, glycofural, isopropyl myristate, isopropyl palmitate, oleic acid, polyethylene glycol, propylene carbonate, and triethyl citrate. The most preferred solvents are N-methyl-2-pyrrolidone, 2-pyrrolidone, dimethyl sulfoxide, triacetin, and propylene carbonate because of the solvating ability and their compatibility.

Additionally, formulations comprising B Lymphocyte Stimulator antagonists and a biodegradable polymer may also include release-rate modification agents and/or pore-forming agents. Examples of release-rate modification agents include, but are not limited to, fatty acids, triglycerides, other like hydrophobic compounds, organic solvents, plasticizing compounds and hydrophilic compounds. Suitable release rate modification agents include, for example, esters of mono-, di-, and tricarboxylic acids, such as 2-ethoxyethyl acetate, methyl acetate, ethyl acetate, diethyl phthalate, dimethyl phthalate, dibutyl phthalate, dimethyl adipate, dimethyl succinate, dimethyl oxalate, dimethyl citrate, triethyl citrate, acetyl tributyl citrate, acetyl triethyl citrate, glycerol triacetate, di(n-butyl) sebecate, and the like; polyhydroxy alcohols, such as propylene glycol, polyethylene glycol, glycerin, sorbitol, and the like; fatty acids; triesters of glycerol, such as triglycerides, epoxidized soybean oil, and other epoxidized vegetable oils; sterols, such as cholesterol; alcohols, such as C.sub.6-C.sub.12 alkanols, 2-ethoxyethanol, and the like. The release rate modification agent may be used singly or in combination with other such agents. Suitable combinations of release rate modification agents include, but are not limited to, glycerin/propylene glycol, sorbitol/glycerine, ethylene oxide/propylene oxide, butylene glycol/adipic acid, and the like. Preferred release rate modification agents include, but are not limited to, dimethyl citrate, triethyl citrate, ethyl heptanoate, glycerin, and hexanediol. Suitable pore-forming agents that may be used in the polymer composition include, but are not limited to, sugars such as sucrose and dextrose, salts such as sodium chloride and sodium carbonate, polymers such as hydroxylpropylcellulose, carboxymethylcellulose, polyethylene glycol, and polyvinylpyrrolidone. Solid crystals that will provide a defined pore size, such as salt or sugar, are preferred.

In specific preferred embodiments the B Lymphocyte Stimulator antagonists are formulated using the BEMA™ BioErodible Mucoadhesive System, MCA™ MucoCutaneous Absorption System, SMP™ Solvent MicroParticle System, or BCP™ BioCompatible Polymer System of Atrix Laboratories, Inc. (Fort Collins, Colo.).

Sustained-release compositions also include liposomally entrapped compositions of the invention (see generally, Langer, Science 249:1527-1533 (1990); Treat et al., in Liposomes in the Therapy of Infectious Disease and Cancer, Lopez-Berestein and Fidler (eds.), Liss, New York, pp. 317-327 and 353-365 (1989)). Liposomes containing B Lymphocyte Stimulator antagonists my be prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 0052322; EP 0036676; EP 0088046; EP 0143949; EP 0142641; Japanese Patent Application 83-118008; U.S. Pat. Nos. 4,485,045 and 4,544,545; and EP 0102324. Ordinarily, the liposomes are of the small (about 200-800 Angstroms) unilamellar type in which the lipid content is greater than about 30 mol. percent cholesterol, the selected proportion being adjusted for the optimal B Lymphocyte Stimulator antagonist therapy.

In another embodiment sustained release compositions of the invention include crystal formulations known in the art.

In yet an additional embodiment, the B Lymphocyte Stimulator antagonists are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

For parenteral administration, in one embodiment, the B Lymphocyte Stimulator antagonist is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the B Lymphocyte Stimulator antagonist uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, sucrose, or dextrins; chelating agents such as EDTA; sugar alcohols such as mannitol or sorbitol; counterions such as sodium; preservatives, such as cresol, phenol, chlorobutanol, benzyl alcohol and parabens, and/or nonionic surfactants such as polysorbates, poloxamers, or PEG.

The B Lymphocyte Stimulator antagonist is typically formulated in such vehicles at a concentration of about 0.001 mg/ml to 250 mg/ml, or 0.1 mg/ml to 100 mg/ml, preferably 1-10 mg/ml or 1-10 mg/ml, at a pH of about 3 to 10, or 3 to 8, more preferably 5-8, most preferably 6-7. It will be understood that the use of certain of the foregoing excipients, carriers, or stabilizers will result in the formation of salts.

The B Lymphocyte Stimulator antagonists to be used for therapeutic administration must be sterile. Sterility is readily accomplished by filtration through sterile filtration membranes (e.g., 0.2 micron membranes). Therapeutic B Lymphocyte Stimulator antagonists generally are placed into a container having a sterile access port, for example, an intravenous solution bag or vial having a stopper pierceable by a hypodermic injection needle.

B Lymphocyte Stimulator antagonists ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous B Lymphocyte Stimulator antagonist solution, and the resulting mixture is lyophilized. The infusion solution is prepared by reconstituting the lyophilized B Lymphocyte Stimulator antagonist using bacteriostatic Water-for-Injection.

Alternatively, the B Lymphocyte Stimulator antagonist is stored in single dose containers in lyophilized form. The infusion selection is reconstituted using a sterile carrier for injection.

The invention also provides a pharmaceutical pack or kit comprising one or more containers filled with one or more of the ingredients of the pharmaceutical compositions of the invention, e.g., a B Lymphocyte Stimulator antagonist. Optionally, associated with such container(s) is a notice in the form prescribed by a governmental agency regulating the manufacture, use or sale of pharmaceuticals or biological products, which notice reflects approval by the agency of manufacture, use or sale for human administration.

Therapeutic and/or Prophylactic Administration and Composition

The invention provides methods of treatment, inhibition and prophylaxis by administration to a subject an effective amount of the B Lymphocyte Stimulator antagonist, preferably an anti-B Lymphocyte Stimulator antibody, typically in a pharmaceutical composition. In a preferred embodiment, the B Lymphocyte Stimulator antagonist is substantially purified (e.g., substantially free from substances that limit its effect or produce undesired side effects). The subject is preferably an animal, including but not limited to, a mammal, such as a rabbit, goat, guinea pig, camel, horse, mouse, rat, hamster, pig, micro-pig, chicken, goat, cow, sheep, dog, cat, non-human primate, and human. In most preferred embodiments, the subject is a human.

Various delivery systems are known and can be used to administer a B Lymphocyte Stimulator antagonist, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce B Lymphocyte Stimulator antagonists into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer B Lymphocyte Stimulator antagonists locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, care must be taken to use materials to which the protein does not absorb.

In another embodiment, B Lymphocyte Stimulator antagonists can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, B Lymphocyte Stimulator antagonists can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Press, Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the B Lymphocyte Stimulator antagonist is a nucleic acid encoding a protein or antibody, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The dosage of the B Lymphocyte Stimulator antagonist (e.g., anti-B Lymphocyte Stimulator antibody) administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Since human antibodies generally have a longer half-life within the human body than antibodies from other species due to the immune response to the foreign polypeptides, when the antagonist is an antibody, lower dosages of human antibodies and less frequent administration is often possible. The dosage and frequency of administration of the B Lymphocyte Stimulator antagonist (e.g., anti-B Lymphocyte Stimulator antibody) may be reduced by enhancing uptake and tissue penetration (e.g., into the brain) of the antagonist by modifications such as, for example, lipidation.

The B lymphocyte stimulator antagonists can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Antagonists—Assays and Molecules

B Lymphocyte Stimulator antagonists also can be small organic molecules, peptides, polypeptides (such as proteins sharing significant similarity to a B Lymphocyte Stimulator protein), antibodies (including fragments, analogs, and derivatives thereof as described herein), and nucleic acids encoding antibodies that bind to a B Lymphocyte Stimulator protein and reduce, inhibit, or extinguish B Lymphocyte Stimulator activity (e.g., the proliferation, differentiation, or survival of B cells, or the ability of a B Lymphocyte Stimulator protein to bind a B Lymphocyte Stimulator protein-binding molecule, such as a B Lymphocyte Stimulator receptor molecule).

Other B Lymphocyte Stimulator antagonists include antisense molecules. Antisense technology can be used to control gene expression through antisense DNA or RNA or through triple-helix formation. Antisense techniques are discussed, for example, in Okano, J. Neurochem. 56: 560 (1991); “Oligodeoxynucleotides as Antisense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, J., Neurochem. 56:560 (1991); Oligodeoxynucleotides as Antisense Inhibitors of Gene Expression, CRC Press, Boca Raton, Fla. (1988). Triple helix formation is discussed in, for instance Lee et al., Nucleic Acids Research 6: 3073 (1979); Cooney et al., Science 241: 456 (1988); and Dervan et al., Science 251: 1360 (1991). The methods are based on binding of a polynucleotide to a complementary DNA or RNA. For example, the 5′ coding portion of a polynucleotide that encodes the extracellular domain of the polypeptide of SEQ ID NO:2 may be used to design an antisense RNA oligonucleotide of from about 10 to 40 base pairs in length. A DNA oligonucleotide is designed to be complementary to a region of the gene involved in transcription thereby preventing transcription and the production of B Lymphocyte Stimulator. The antisense RNA oligonucleotide hybridizes to the mRNA in vivo and blocks translation of the mRNA molecule into B Lymphocyte Stimulator polypeptide. The oligonucleotides described above can also be delivered to cells such that the antisense RNA or DNA may be expressed in vivo to inhibit production of B Lymphocyte Stimulator.

In one embodiment, the B Lymphocyte Stimulator antisense nucleic acid of the invention is produced intracellularly by transcription from an exogenous sequence. For example, a vector or a portion thereof, is transcribed, producing an antisense nucleic acid (RNA) of the B Lymphocyte Stimulator. Such a vector would contain a sequence encoding the B Lymphocyte Stimulator antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the sequence encoding B Lymphocyte Stimulator, or fragments thereof, can be by any promoter known in the art to act in vertebrate, preferably human, cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980)), the herpes thymidine promoter (Wagner et al., Proc. Natl. Acad. Sci. U.S.A. 78:1441-1445 (1981)), the regulatory sequences of the metallothionein gene (Brinster, et al., Nature 296:39-42 (1982)), etc.

The antisense nucleic acids comprise a sequence complementary to at least a portion of an RNA transcript of a B Lymphocyte Stimulator gene. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded B Lymphocyte Stimulator antisense nucleic acids, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with a B Lymphocyte Stimulator RNA it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′-non-translated, non-coding regions of the B Lymphocyte Stimulator gene, could be used in an antisense approach to inhibit translation of endogenous B Lymphocyte Stimulator mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of B Lymphocyte Stimulator mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)_{w}, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an alpha-anomeric oligonucleotide. An alpha-anomeric oligonucleotide forms specific double-stranded hybrids with complementary RNA in which, contrary to the usual beta-units, the strands run parallel to each other (Gautier et al., Nucl. Acids Res. 15:6625-6641 (1987)). The oligonucleotide is a 2-O-methylribonucleotide (Inoue et al., Nucl. Acids Res. 15:6131-6148 (1987)), or a chimeric RNA-DNA analogue (Inoue et al., FEBS Lett. 215:327-330 (1997)).

Polynucleotides may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (Nucl. Acids Res. 16:3209 (1988)), methylphosphonate oligonucleotides can be prepared by use of controlled pore glass polymer supports (Sarin et al., Proc. Natl. Acad. Sci. U.S.A. 85:7448-7451 (1988)), etc.

While antisense nucleotides complementary to the B Lymphocyte Stimulator coding region sequence could be used, those complementary to the transcribed untranslated region are most preferred.

B Lymphocyte Stimulator antagonists also include antibodies specific for B Lymphocyte Stimulator receptors or the B Lymphocyte Stimulator. Antagonistic antibodies may be prepared by any of a variety of standard methods using B Lymphocyte Stimulator or B Lymphocyte Stimulator receptor immunogens. B Lymphocyte Stimulator immunogens include the complete B Lymphocyte Stimulator polypeptide sequence (SEQ ID NO:2) and B Lymphocyte Stimulator polypeptide fragments comprising, for example, the ligand binding domain, TNF-conserved domain, extracellular domain, transmembrane domain, and/or intracellular domain, or any combination thereof.

Polyclonal and monoclonal antibody antagonists can be raised according to the methods disclosed in Tartaglia and Goeddel, J. Biol. Chem. 267(7):4304-4307 (1992)); Tartaglia et al., Cell 73:213-216 (1993)), and International Patent Application Publication WO 94/09137 and are preferably specific to (i.e., bind to B Lymphocyte Stimulator protein or fragments thereof). The term “antibody” (Ab) or “monoclonal antibody” (mAb) as used herein is meant to include intact molecules as well as fragments thereof (such as, for example, Fab and F(ab′) fragments) which are capable of binding an antigen. Fab, Fab′ and F(ab′) fragments lack the Fc fragment of an intact antibody, clear more rapidly from the circulation, and may have less non-specific tissue binding of an intact antibody (Wahl et al., J. Nucl. Med., 24:316-325 (1983)).

In a preferred method, antagonistic antibodies are mAbs. Such mAbs can be prepared using hybridoma technology (Kohler and Millstein, Nature 256:495-497 (1975) and U.S. Pat. No. 4,376,110; Harlow et al., Antibodies: A Laboratory Manual, Cold Spring Harbor Laboratory Press, Cold Spring Harbor, N.Y., 1988; Monoclonal Antibodies and Hybridomas: A New Dimension in Biological Analyses, Plenum Press, New York, N.Y., 1980; Campbell, “Monoclonal Antibody Technology,” In: Laboratory Techniques in Biochemistry and Molecular Biology, Volume 13 (Burdon et al., eds.), Elsevier, Amsterdam (1984)).

Proteins and other compounds which bind B Lymphocyte Stimulator are also candidate antagonists useful in the context of the invention. Such binding compounds can be “captured” using the yeast two-hybrid system (Fields and Song, Nature 340:245-246 (1989)). A modified version of the yeast two-hybrid system has been described by Roger Brent and his colleagues (Gyuris, Cell 75:791-803 (1993); Zervos et al., Cell 72:223-232 (1993)). Preferably, the yeast two-hybrid system is used to capture compounds which bind to B Lymphocyte Stimulator protein or fragments thereof, including the ligand binding domain, TNF-conserved domain, extracellular domain, transmembrane domain, or intracellular domain, or any combination thereof. Such compounds are good candidate antagonists.

In addition, using the two-hybrid assay described above, the extracellular or intracellular domain of the B Lymphocyte Stimulator receptor, or a portion thereof, may also be used to identify cellular proteins which interact with the B Lymphocyte Stimulator receptor in vivo. Such an assay may also be used to identify ligands with potential antagonistic activity of B Lymphocyte Stimulator receptor function. This screening assay has previously been used to identify protein which interact with the cytoplasmic domain of the murine TNF-RII and led to the identification of two receptor associated proteins. Rothe et al., Cell 78:681 (1994). Such proteins and amino acid sequences which bind to the cytoplasmic domain of the B Lymphocyte Stimulator receptors are good candidate antagonists of the invention.

Other screening techniques include the use of cells which express a B Lymphocyte Stimulator receptor (for example, transfected CHO cells) in a system which measures extracellular pH changes caused by receptor activation, for example, as described in Science, 246:181-296 (1989). In another example, potential antagonists may be contacted with a cell which expresses a B Lymphocyte Stimulator receptor and a second messenger response, e.g., signal transduction may be measured to determine whether the potential antagonist is effective.

### Antibodies

In one embodiment, the B Lymphocyte Stimulator antagonist is an anti-B Lymphocyte Stimulator antibody which neutralizes B Lymphocyte Stimulator biological activity. Anti-B Lymphocyte Stimulator antibodies may bind, for example, soluble B Lymphocyte Stimulator protein, membrane-bound B Lymphocyte Stimulator protein, recombinant B Lymphocyte Stimulator protein purified from a cell culture wherein said recombinant B Lymphocyte Stimulator protein is encoded by a polynucleotide encoding at least amino acids 134 to 285 of SEQ ID NO:2, and/or recombinant B Lymphocyte Stimulator protein encoded by a polynucleotide encoding at least amino acids 134 to 285 of SEQ ID NO:2.

The B Lymphocyte Stimulator antagonist also includes antibodies which disrupt either partially or fully the interactions between B Lymphocyte Stimulator protein and at least one of its receptors. The B Lymphocyte Stimulator antagonist includes both receptor-specific antibodies and ligand-specific antibodies (i.e., anti-B Lymphocyte Stimulator antibodies). Included are receptor-specific antibodies which do not prevent ligand binding but prevent receptor activation. Receptor activation (i.e., signaling) may be determined by techniques described herein or otherwise known in the art. Also included are receptor-specific antibodies which both prevent ligand binding and receptor activation. Likewise, included are neutralizing antibodies which bind the ligand and prevent binding of the ligand to the receptor, as well as antibodies which bind the ligand, thereby preventing receptor activation, but do not prevent the ligand from binding the receptor. Further included are antibodies that bind to B Lymphocyte Stimulator irrespective of whether B Lymphocyte Stimulator is bound to a B Lymphocyte Stimulator receptor.

Several antagonistic monoclonal antibodies have been generated against B Lymphocyte Stimulator protein, as previously described and hereby incorporated by reference (see, e.g., U.S. Pat. No. 7,317,089 and U.S. Patent Application Publications 2009/0104189, 2008/0050381, and 2005/0255532). In one embodiment, the B Lymphocyte Stimulator antagonist is any of the anti-B Lymphocyte Stimulator antibodies described in U.S. Pat. No. 7,317,089 and U.S. Patent Application Publications 2009/0104189, 2008/0050381, and 2005/0255532. In a preferred embodiment, the anti-B Lymphocyte Stimulator antibody is BENLYSTA® (belimumab) from Human Genome Sciences, Inc. In another embodiment, the anti-B Lymphocyte Stimulator antibody is LY2127399 (Eli Lilly and Co., Indianapolis, Ind.). In another embodiment, the anti-B Lymphocyte Stimulator antibody is the antibody disclosed/claimed in U.S. Pat. No. 7,317,089.

In another embodiment, B Lymphocyte Stimulator antagonist is any of the anti-B Lymphocyte Stimulator antibodies having one or more of the same biological characteristics as one or more of the antibodies described in U.S. Pat. No. 7,317,089 and U.S. Patent Application Publications 2009/0104189, 2008/0050381, and 2005/0255532, or the anti-B Lymphocyte Stimulator antibody BENLYSTA® (belimumab), from Human Genome Sciences, Inc., or the anti-B Lymphocyte Stimulator antibody LY2127399 (from Eli Lilly and Co., Indianapolis, Ind.). By “biological characteristics” is meant, the in vitro or in vivo activities or properties of these previously described antibodies, such as, for example, the ability to bind to B Lymphocyte Stimulator protein (e.g., the polypeptide of SEQ ID NO:2, the mature form of B Lymphocyte Stimulator protein, the membrane-bound form of B Lymphocyte Stimulator protein, the soluble form of B Lymphocyte Stimulator protein (amino acids 134 to 285 of SEQ ID NO:2), and an antigenic and/or epitope region of B Lymphocyte Stimulator protein), the ability to substantially block B Lymphocyte Stimulator/B Lymphocyte Stimulator receptor binding, or the ability to block B Lymphocyte Stimulator mediated biological activity (e.g., stimulation of B cell proliferation and immunoglobulin production). Optionally, the B Lymphocyte Stimulator antagonist is any anti-B Lymphocyte Stimulator antibody that will bind to the same epitope as at least one of the antibodies previously described in U.S. Pat. No. 7,317,089 and U.S. Patent Application Publications 2009/0104189, 2008/0050381, and 2005/0255532 or specifically referred to herein, including the anti-B Lymphocyte Stimulator antibody BENLYSTA® (belimumab), from Human Genome Sciences, Inc., and the anti-B Lymphocyte Stimulator antibody LY2127399 (Eli Lilly and Co., Indianapolis, Ind.). Such epitope binding can be routinely determined using assays known in the art.

In another embodiment, anti-B Lymphocyte Stimulator antibodies specifically bind only the soluble form of B Lymphocyte Stimulator protein.

Anti-B Lymphocyte Stimulator antibodies may also bind both the membrane-bound and soluble form of B Lymphocyte Stimulator.

As described above, anti-B Lymphocyte Stimulator antibodies include antibodies that inhibit or reduce the ability of B Lymphocyte Stimulator to bind B Lymphocyte Stimulator receptor in vitro and/or in vivo. In a specific embodiment, anti-B Lymphocyte Stimulator antibodies inhibit or reduce the ability of B Lymphocyte Stimulator to bind B Lymphocyte Stimulator receptor in vitro. In another nonexclusive specific embodiment, anti-B Lymphocyte Stimulator antibodies inhibit or reduce the ability of B Lymphocyte Stimulator to bind B Lymphocyte Stimulator receptor in vivo. Such inhibition can be assayed using techniques described herein or otherwise known in the art.

As described above, anti-B Lymphocyte Stimulator antibodies include antibodies that inhibit or reduce a B Lymphocyte Stimulator-mediated biological activity in vitro and/or in vivo. In a specific embodiment, anti-B Lymphocyte Stimulator antibodies inhibit or reduce B Lymphocyte Stimulator-mediated B cell proliferation in vitro. Such inhibition can be assayed by routinely modifying B cell proliferation assays known in the art. In another nonexclusive specific embodiment, anti-B Lymphocyte Stimulator antibodies inhibit or reduce B Lymphocyte Stimulator-mediated B cell proliferation in vivo. In a specific embodiment, the anti-B Lymphocyte Stimulator antibody is 15C10, as described in U.S. Patent Application Publication 2009/0104189 or a humanized form thereof. In another preferred specific embodiment, the anti-B Lymphocyte Stimulator antibody is 16C9, as described in U.S. Patent Application Publication 2009/0104189, or a humanized form thereof. Thus, in specific embodiments of the invention, a 16C9 and/or 15C10 antibody, or humanized forms thereof, are used to bind soluble B Lymphocyte Stimulator and thereby inhibit (either partially or completely) B cell proliferation. In another preferred specific embodiment, the anti-B Lymphocyte Stimulator antibody is BENLYSTA® (belimumab) from Human Genome Sciences, Inc. In another preferred specific embodiment, the anti-B Lymphocyte Stimulator antibody is LY2127399 (Eli Lilly and Co., Indianapolis, Ind.). In another preferred specific embodiment, the anti-B Lymphocyte Stimulator antibody is 4H4, as described in U.S. Patent Application Publication 2008/0050381.

As described above, the B Lymphocyte Stimulator antagonist includes anti-B Lymphocyte Stimulator antibodies that specifically bind to the same epitope as at least one of the antibodies specifically referred to herein, in vitro and/or in vivo, including the anti-B Lymphocyte Stimulator antibody BENLYSTA® (belimumab), from Human Genome Sciences, Inc., and the anti-B Lymphocyte Stimulator antibody LY2127399 (Eli Lilly and Co., Indianapolis, Ind.).

In a specific embodiment, the B Lymphocyte Stimulator antagonist includes anti-B Lymphocyte Stimulator antibodies that specifically bind to an amino acid sequence contained in amino acid residues from about Ala-134 to about Cys-146 of SEQ ID NO:2, in vitro. In another specific, non-exclusive embodiment, the B Lymphocyte Stimulator antagonist includes anti-B Lymphocyte Stimulator antibodies that bind to an amino acid sequence contained in amino acid residues from about Ala-134 to about Cys-146 of SEQ ID NO:2, in vivo. In a specific embodiment, the B Lymphocyte Stimulator antagonist includes anti-B Lymphocyte Stimulator antibodies that specifically bind to an amino acid sequence contained in amino acid residues from about Ser-171 to about Phe-194 of SEQ ID NO:2, in vitro. In another specific, non-exclusive embodiment, the B Lymphocyte Stimulator antagonist includes anti-B Lymphocyte Stimulator antibodies that bind to an amino acid sequence contained in amino acid residues from about Ser-171 to about Phe-194 of SEQ ID NO:2, in vivo. In another specific, non-exclusive embodiment, the B Lymphocyte Stimulator antagonist includes anti-B Lymphocyte Stimulator antibodies that bind to an amino acid sequence contained in amino acid residues from Lys-173 to Lys-188 of SEQ ID NO:2, in vitro. In another specific, non-exclusive embodiment, the B Lymphocyte Stimulator antagonist includes anti-B Lymphocyte Stimulator antibodies that specifically bind to an amino acid sequence contained in amino acid residues from Lys-173 to Lys-188 of SEQ ID NO:2, in vivo.

In an additional specific embodiment, the B Lymphocyte Stimulator antagonist includes anti-B Lymphocyte Stimulator antibodies that specifically bind to an amino acid sequence contained in amino acid residues from about Glu-223 to about Tyr-246 of SEQ ID NO:2, in vitro. In another specific, non-exclusive embodiment, the B Lymphocyte Stimulator antagonist includes anti-B Lymphocyte Stimulator antibodies that specifically bind to an amino acid sequence contained in amino acid residues from about Glu-223 to about Tyr-246 of SEQ ID NO:2, in vivo. In another specific, non-exclusive embodiment, the B Lymphocyte Stimulator antagonist includes anti-B Lymphocyte Stimulator antibodies that specifically bind to an amino acid sequence contained in amino acid residues from Val-227 to Asn-242 of SEQ ID NO:2, in vitro. In another specific, non-exclusive embodiment, the B Lymphocyte Stimulator antagonist includes anti-B Lymphocyte Stimulator antibodies that specifically bind to an amino acid sequence contained in amino acid residues from Val-227 to Asn-242 of SEQ ID NO:2, in vivo. In another specific, non-exclusive embodiment, the B Lymphocyte Stimulator antagonist includes anti-B Lymphocyte Stimulator antibodies that specifically bind to an amino acid sequence contained in amino acid residues from Phe-230 to Cys-245 of SEQ ID NO:2, in vitro. In another specific, non-exclusive embodiment, the B Lymphocyte Stimulator antagonist includes anti-B Lymphocyte Stimulator antibodies that specifically bind to an amino acid sequence contained in amino acid residues from Phe-230 to Cys-245 of SEQ ID NO:2, in vivo.

The B Lymphocyte Stimulator antagonist also includes anti-B Lymphocyte Stimulator antibodies that competitively inhibit the binding of any of the anti-B Lymphocyte Stimulator antibodies previously described in U.S. Pat. No. 7,317,089 and U.S. Patent Application Publications 2009/0104189, 2008/0050381, and 2005/0255532, or specifically referred to herein, including the anti-B Lymphocyte Stimulator antibody BENLYSTA® (belimumab), from Human Genome Sciences, Inc., and the anti-B Lymphocyte Stimulator antibody LY2127399 (Eli Lilly and Co., Indianapolis, Ind.). Competitive inhibition can be determined by any method known in the art, for example, using the competitive binding assays described herein. In preferred embodiments, the antibody competitively inhibits the binding of any of the anti-B Lymphocyte Stimulator antibodies previously described in U.S. Pat. No. 7,317,089 and U.S. Patent Application Publications 2009/0104189, 2008/0050381, and 2005/0255532, or specifically referred to herein, including the anti-B Lymphocyte Stimulator antibody BENLYSTA® (belimumab), from Human Genome Sciences, Inc., and the anti-B Lymphocyte Stimulator antibody LY2127399 (Eli Lilly and Co., Indianapolis, Ind.) by at least 95%, at least 90%, at least 85%, at least 80%, at least 75%, at least 70%, at least 60%, at least 50%, to the polypeptide of SEQ ID NO:2, or a polypeptide comprising amino acid residues 134-285 of SEQ ID NO:2.

The B Lymphocyte Stimulator antagonist also includes anti-B Lymphocyte Stimulator antibodies comprising the VH and VL domains of scFvs described in U.S. Patent Application Publication 2005/0255532, which is hereby incorporated by reference.

Anti-B Lymphocyte Stimulator antibodies (including molecules comprising, or alternatively consisting of, antibody fragments or variants thereof) that immunospecifically bind to a polypeptide or a polypeptide fragment of B Lymphocyte Stimulator can comprise, or alternatively consist of, a polypeptide having the amino acid sequence of any one, two, three or more of the VH complementarity determining regions (“CDRs”) (i.e., VH CDR1, VH CDR2, or VH CDR3) described in U.S. Pat. No. 7,220,840 and/or any one, two, three or more of the VL CDRs (i.e., VL CDR1, VL CDR2, or VL CDR3) described in U.S. Pat. No. 7,220,840. In one embodiment, the antibodies comprise, or alternatively consist of, a polypeptide having the amino acid sequence of any one of the VH CDR1s described in U.S. Pat. No. 7,220,840 and/or any one of the VL CDR1s described in U.S. Pat. No. 7,220,840. In another embodiment, the antibodies comprise, or alternatively consist of, a polypeptide having the amino acid sequence of any one of the VH CDR2s described in U.S. Pat. No. 7,220,840 and/or any one of the VL CDR2s described in U.S. Pat. No. 7,220,840. In a preferred embodiment, the antibodies comprise, or alternatively consist of, a polypeptide having the amino acid sequence of any one of the VH CDR3s described in U.S. Pat. No. 7,220,840 and/or any one of the VL CDR3s described in U.S. Pat. No. 7,220,840. Molecules comprising, or alternatively consisting of, fragments or variants of these antibodies (e.g., including VH domains, VH CDRs, VL domains, or VL CDRs having an amino acid sequence of any one of those described in U.S. Pat. No. 7,220,840), that immunospecifically bind the soluble form of B Lymphocyte Stimulator, the membrane-bound form of B Lymphocyte Stimulator, and/or both the soluble form and membrane-bound form of B Lymphocyte Stimulator, are also encompassed by the invention, as are nucleic acid molecules that encode these antibodies, and/or molecules.

In another embodiment, anti-B Lymphocyte Stimulator antibodies (including molecules comprising, or alternatively consisting of, antibody fragments or variants thereof) immunospecifically bind to a polypeptide or polypeptide fragment of B Lymphocyte Stimulator, and comprise, or alternatively consist of, a polypeptide having the amino acid sequence of any one of the VH CDR1s described in U.S. Pat. No. 7,220,840, any one of the VH CDR2s described in U.S. Pat. No. 7,220,840, and/or any one of the VH CDR3s described in U.S. Pat. No. 7,220,840. In another embodiment, the antibodies comprise, or alternatively consist of, a polypeptide having the amino acid sequence of any one of the VL CDR1s described in U.S. Pat. No. 7,220,840, any one of the VL CDR2s described in U.S. Pat. No. 7,220,840, and/or any one of the VL CDR3s described in U.S. Pat. No. 7,220,840. In a preferred embodiment, the antibodies comprise, or alternatively consist of, at least one, two, three, four, five, six, or more CDRs that correspond to the same scFv described in U.S. Pat. No. 7,220,840, more preferably where CDR1, CDR2, and CDR3 of the VL domain correspond to the same scFv or where CDR1, CDR2, and CDR3 of the VH domain correspond to the same scFv, and most preferably where all six CDRs correspond to the same scFv described in U.S. Pat. No. 7,220,840. Molecules comprising, or alternatively consisting of, fragments or variants of these antibodies (e.g., including VH domains, VH CDRs, VL domains, or VL CDRs having an amino acid sequence of any one of those described in U.S. Pat. No. 7,220,840), that immunospecifically bind the soluble form of B Lymphocyte Stimulator, the membrane-bound form of B Lymphocyte Stimulator, and/or both the soluble form and membrane-bound form of B Lymphocyte Stimulator, are also encompassed by the invention, as are nucleic acid molecules that encode these antibodies, and/or molecules.

Anti-B Lymphocyte Stimulator antibodies (including molecules comprising, or alternatively consisting of, antibody fragments or variants thereof) that immunospecifically bind to a polypeptide or a polypeptide fragment of B Lymphocyte Stimulator can comprise or alternatively consist of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of an anti-B Lymphocyte Stimulator antibody or antibody fragment thereof, including a VH domain, VHCDR, VL domain, or VLCDR, described in U.S. Pat. No. 7,220,840. Nucleic acid molecules encoding these antibodies are also encompassed by the invention.

In another embodiment, an anti-B Lymphocyte Stimulator antibody (including a molecule comprising, or alternatively consisting of, an antibody fragment or variant thereof), that immunospecifically binds to B Lymphocyte Stimulator comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VH domains described in U.S. Pat. No. 7,220,840. In another embodiment, the antibody that immunospecifically binds to B Lymphocyte Stimulator comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VH CDRs described in U.S. Pat. No. 7,220,840. In another embodiment, the antibody that immunospecifically binds to B Lymphocyte Stimulator comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to any one of the VH CDR3s described in U.S. Pat. No. 7,220,840. Nucleic acid molecules encoding these antibodies are also encompassed by the invention.

In another embodiment, an anti-B Lymphocyte Stimulator antibody (including a molecule comprising, or alternatively consisting of, an antibody fragment or variant thereof) that immunospecifically binds to B Lymphocyte Stimulator comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VL domains described in U.S. Pat. No. 7,220,840. In another embodiment, the antibody that immunospecifically binds to B Lymphocyte Stimulator comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VL CDRs described in U.S. Pat. No. 7,220,840. In another embodiment, the antibody that immunospecifically binds to B Lymphocyte Stimulator comprises, or alternatively consists of, a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to any one of the VL CDR3s described in U.S. Pat. No. 7,220,840. Nucleic acid molecules encoding these antibodies are also encompassed by the invention.

In other preferred embodiments, an anti-B Lymphocyte Stimulator antibody competitively inhibits binding of an antibody comprising a fragment (e.g., VH domain, VL domain, VHCDR1, VHCDR2, VHCDR3, VLCDR1, VLCDR2, or VLCDR3) or variant of an scFv described in U.S. Pat. No. 7,220,840 to a B Lymphocyte Stimulator polypeptide. In preferred embodiments, the anti-B Lymphocyte Stimulator antibody reduces the binding of an antibody comprising a fragment (e.g., VH domain, VL domain, VHCDR1, VHCDR2, VHCDR3, VLCDR1, VLCDR2, or VLCDR3) or variant of an scFv described in U.S. Pat. No. 7,220,840 to a B Lymphocyte Stimulator polypeptide by between 1% and 10% in a competitive inhibition assay. In preferred embodiments, the anti-B Lymphocyte Stimulator antibody reduces the binding of an antibody comprising a fragment (e.g., VH domain, VL domain, VHCDR1, VHCDR2, VHCDR3, VLCDR1, VLCDR2, or VLCDR3) or variant of an scFv described in U.S. Pat. No. 7,220,840 to a B Lymphocyte Stimulator polypeptide by between 1% and 10% in a competitive inhibition assay.

Cell lines that express anti-B Lymphocyte Stimulator antibodies that comprise the VH and VL domains of scFvs, as described in U.S. Patent Application Publication 2005/0255532, have been deposited with the American Type Culture Collection (“ATCC™”) on the dates listed in Table 2 and given the ATCC™ Deposit Numbers identified in Table 2. The ATCC™ is located at 10801 University Boulevard, Manassas, Va. 20110-2209, USA. The ATCC™ deposit was made pursuant to the terms of the Budapest Treaty on the international recognition of the deposit of microorganisms for purposes of patent procedure.

TABLE 2
ATCC ™ Deposit Information.

| | | ATCC ™ | |
|---|---|---|---|
| | Corresponding | Deposit | ATCC ™ |
| Cell Line | scFv | Number | Deposit Date |
| NSO-B11-15 | I050B11-15 | PTA-3238 | Mar. 27, 2001 |
| NSO-anti-B | | | |
| Lymphocyte | I006D08 | PTA-3239 | Mar. 27, 2001 |
| Stimulator-6D08-18 | | | |
| NSO-anti-B | | | |
| Lymphocyte | I116A01 | PTA-3240 | Mar. 27, 2001 |
| Stimulator-116A01-60 | | | |
| IO26C04K | I026C04-K | PTA-3241 | Mar. 27, 2001 |
| IO50A12 | I050A12 | PTA-3242 | Mar. 27, 2001 |
| IO50-B11 | I050B11 | PTA-3243 | Mar. 27, 2001 |

Accordingly, in one embodiment, the B Lymphocyte Stimulator antagonist is an anti-B Lymphocyte Stimulator antibody that comprises the VH and VL domains of an scFv disclosed in U.S. Patent Application Publication 2005/0255532.

In a preferred embodiment, the B Lymphocyte Stimulator antagonist is the anti-B Lymphocyte Stimulator antibody expressed by cell line NSo-B11-15.

In a preferred embodiment, the B Lymphocyte Stimulator antagonist is the anti-B Lymphocyte Stimulator antibody expressed by cell line NSO-anti-B Lymphocyte Stimulator-6D08-18.

In a preferred embodiment, the B Lymphocyte Stimulator antagonist is the anti-B Lymphocyte Stimulator antibody expressed by cell line NSO-anti-B Lymphocyte Stimulator-116A01-60.

In a preferred embodiment, the B Lymphocyte Stimulator antagonist is the anti-B Lymphocyte Stimulator antibody expressed by cell line IO26C04K.

In a preferred embodiment, the B Lymphocyte Stimulator antagonist is the anti-B Lymphocyte Stimulator antibody expressed by cell line IO50A12.

In a preferred embodiment, the B Lymphocyte Stimulator antagonist is the anti-B Lymphocyte Stimulator antibody expressed by cell line NSO-B11.

In a specific embodiment, the specific antibodies described above are humanized using techniques described herein or otherwise known in the art and then used as therapeutics as described herein.

In another specific embodiment, any of the antibodies listed above are used in a soluble form.

In another specific embodiment, any of the antibodies listed above are conjugated to a toxin or a label (as described infra). Such conjugated antibodies are used to kill a particular population of cells or to quantitate a particular population of cells. In a preferred embodiment, such conjugated antibodies are used to kill B cells expressing B Lymphocyte Stimulator receptor on their surface.

As discussed above, antibodies to B Lymphocyte Stimulator can, in turn, be utilized to generate anti-idiotype antibodies that “mimic” B Lymphocyte Stimulator, using techniques well known to those skilled in the art. (See, e.g., Greenspan & Bona, FASEB J. 7(5):437-444 (1989), and Nissinoff, J. Immunol. 147(8):2429-2438 (1991)). For example, antibodies which bind to B Lymphocyte Stimulator and competitively inhibit the B Lymphocyte Stimulator multimerization and/or binding to ligand can be used to generate anti-idiotypes that “mimic” the B Lymphocyte Stimulator TNF multimerization and/or binding domain and, as a consequence, bind to and neutralize B Lymphocyte Stimulator and/or its ligand. Such neutralizing anti-idiotypes or Fab fragments of such anti-idiotypes can be used in therapeutic regimens to neutralize B Lymphocyte Stimulator ligand. For example, such anti-idiotypic antibodies can be used to bind B Lymphocyte Stimulator, or to bind B Lymphocyte Stimulator receptors on the surface of cells of B cell lineage, and thereby block B Lymphocyte Stimulator mediated B cell activation, proliferation, and/or differentiation.

In a preferred embodiment, the B Lymphocyte Stimulator antagonist is an antagonistic antibody that binds B Lymphocyte Stimulator polypeptides comprising, or alternatively, consisting of, a contiguous sequence of amino acid residues at least 80%, 85%, 90%, 92%, 95%, 96%, 97%, 98% or 99% identical to the amino acid sequence of SEQ ID NO:2. In specific embodiments, the antagonistic antibody binds homomeric, especially homotrimeric, B Lymphocyte Stimulator polypeptides. In other specific embodiments, the antagonistic antibodies immunospecifically bind heteromeric, especially heterotrimeric, B Lymphocyte Stimulator polypeptides such as a heterotrimer containing two B Lymphocyte Stimulator polypeptides and one APRIL polypeptide (e.g., SEQ ID NO:20 or SEQ ID NO:47) or a heterotrimer containing one B Lymphocyte Stimulator polypeptide and two APRIL polypeptides.

Immunospecific binding excludes non-specific binding but does not necessarily exclude cross-reactivity with other antigens. Antigenic epitopes need not necessarily be immunogenic. The antagonistic antibodies useful in the invention may be assayed for immunospecific binding to B Lymphocyte Stimulator and cross-reactivity with other antigens by any method known in the art. In particular, the ability of an antibody to immunospecifically bind to the soluble form or membrane-bound form of B Lymphocyte Stimulator and the specificity of the antibody, fragment, or variant for B Lymphocyte Stimulator polypeptide from a particular species (e.g., murine, monkey or human, preferably human) may be determined using or routinely modifying techniques described herein or otherwise known in art.

Immunoassays which can be used to analyze immunospecific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Unless otherwise defined in the specification, specific binding or immunospecific binding by an anti-B Lymphocyte Stimulator antibody means that the anti-B Lymphocyte Stimulator antibody binds B Lymphocyte Stimulator but does not significantly bind to (i.e., cross react with) proteins other than B Lymphocyte Stimulator, such as other proteins in the same family of proteins, e.g., other TNF family ligands. An antibody that binds B Lymphocyte Stimulator protein and does not cross-react with other proteins is not necessarily an antibody that does not bind said other proteins in all conditions; rather, the B Lymphocyte Stimulator-specific antibody preferentially binds B Lymphocyte Stimulator compared to its ability to bind said other proteins such that it will be suitable for use in treatment, i.e., result in no unreasonable adverse effects in treatment. It is well known that the portion of a protein bound by an antibody is known as the epitope. An epitope may either be linear (i.e., comprised of sequential amino acids residues in a protein sequences) or conformational (i.e., comprised of one or more amino acid residues that are not contiguous in the primary structure of the protein but that are brought together by the secondary, tertiary or quaternary structure of a protein). Given that B Lymphocyte Stimulator-specific antibodies bind to epitopes of B Lymphocyte Stimulator, an antibody that specifically binds B Lymphocyte Stimulator may or may not bind fragments of B Lymphocyte Stimulator and/or variants of B Lymphocyte Stimulator (e.g., proteins that are at least 90% identical to B Lymphocyte Stimulator) depending on the presence or absence of the epitope bound by a given B Lymphocyte Stimulator-specific antibody in the B Lymphocyte Stimulator fragment or variant. Likewise, B Lymphocyte Stimulator-specific antibodies of the invention may bind species orthologues of B Lymphocyte Stimulator (including fragments thereof) depending on the presence or absence of the epitope recognized by the antibody in the orthologue. Additionally, B Lymphocyte Stimulator-specific antibodies of the invention may bind modified forms of B Lymphocyte Stimulator, for example, B Lymphocyte Stimulator fusion proteins. In such a case when antibodies of the invention bind B Lymphocyte Stimulator fusion proteins, the antibody must make binding contact with the B Lymphocyte Stimulator moiety of the fusion protein in order for the binding to be specific. Antibodies that specifically bind to B Lymphocyte Stimulator can be identified, for example, by immunoassays or other techniques known to those of skill in the art.

In particularly preferred embodiments, antagonistic antibodies useful in the context of the invention immunospecifically bind homomeric, especially homotrimeric, B Lymphocyte Stimulator, wherein the individual protein components of the multimers consist of the mature form of B Lymphocyte Stimulator (e.g., amino acids residues 134-285 of SEQ ID NO:2). In other specific embodiments, antagonistic antibodies useful in the context of the invention bind heteromeric, especially heterotrimeric, B Lymphocyte Stimulator polypeptides such as a heterotrimer containing two B Lymphocyte Stimulator polypeptides and one APRIL polypeptide or a heterotrimer containing one B Lymphocyte Stimulator polypeptide and two APRIL polypeptides, and wherein the individual protein components of the B Lymphocyte Stimulator heteromer consist of the mature extracellular soluble portion of either B Lymphocyte Stimulator or the mature extracellular soluble portion of APRIL.

In specific embodiments, the antagonistic antibodies bind conformational epitopes of a B Lymphocyte Stimulator monomeric protein. In specific embodiments, the antagonistic antibodies of the invention bind conformational epitopes of a B Lymphocyte Stimulator multimeric, especially trimeric, protein. In other embodiments, antagonistic antibodies bind conformational epitopes that arise from the juxtaposition of B Lymphocyte Stimulator with a heterologous polypeptide, such as might be present when B Lymphocyte Stimulator forms heterotrimers (e.g., with APRIL polypeptides (e.g., SEQ ID NO:20 or SEQ ID NO:47)), or in fusion proteins between B Lymphocyte Stimulator and a heterologous polypeptide.

In one embodiment, antagonistic antibodies immunospecifically bind a B Lymphocyte Stimulator polypeptide having the amino acid sequence of SEQ ID NO:2 or as encoded by the cDNA clone contained in ATCC™ Deposit No. 97768, or a polypeptide comprising a portion (i.e., a fragment) of the above polypeptides. In another embodiment, antagonistic antibodies bind an isolated B Lymphocyte Stimulator polypeptide having the amino acid sequence of SEQ ID NO:19 or the amino acid sequence encoded by the cDNA clone contained in ATCC™ Deposit No. 203518, or an antibody that binds polypeptide comprising a portion (i.e., fragment) of the above polypeptides.

Antagonistic antibodies useful in the context of the invention bind B Lymphocyte Stimulator polypeptides as isolated polypeptides, in their naturally occurring state and/or their native conformation. By “isolated polypeptide” is intended a polypeptide removed from its native environment. Thus, a polypeptide produced by and/or contained within a recombinant host cell is considered isolated for purposes of the invention. Also intended as an “isolated polypeptide” are polypeptides that have been purified, partially or substantially, from a recombinant host cell. Thus, antagonistic antibodies useful in the context of the invention may bind recombinantly produced B Lymphocyte Stimulator polypeptides.

Antagonistic antibodies useful in the context of the invention may also bind B Lymphocyte Stimulator expressed on the surface of a cell, wherein said B Lymphocyte Stimulator polypeptide is encoded by a polynucleotide encoding amino acids 1 to 285 of SEQ ID NO:2 operably associated with a regulatory sequence that controls expression of said polynucleotide. In certain embodiments, said B Lymphocyte Stimulator polypeptide expressed on the surface of a cell is a recombinant B Lymphocyte Stimulator polypeptide. In other embodiments, said B Lymphocyte Stimulator polypeptide expressed on the surface of the cell is a naturally occurring B Lymphocyte Stimulator polypeptide. As a non-limiting example, an antagonistic antibody useful in the context of the invention may bind a B Lymphocyte Stimulator expressed on the surface of the cell wherein Lys 132 and/or Arg-133 of the B Lymphocyte Stimulator sequence shown in SEQ ID NO:2 is mutated to another amino acid residue, or deleted altogether, thereby preventing or diminishing release of the soluble form of B Lymphocyte Stimulator from cells expressing B Lymphocyte Stimulator.

Antagonistic antibodies useful in the context of the invention may also bind B Lymphocyte Stimulator secreted by a cell, wherein said B Lymphocyte Stimulator polypeptide is encoded by a polynucleotide encoding amino acids 1 to 285 of SEQ ID NO:2 operably associated with a regulatory sequence that controls expression of said polynucleotide. In certain embodiments, said B Lymphocyte Stimulator polypeptide secreted by a cell is a recombinant B Lymphocyte Stimulator polypeptide. In other embodiments, said B Lymphocyte Stimulator polypeptide secreted by a cell is a naturally occurring B Lymphocyte Stimulator polypeptide.

Antagonistic antibodies useful in the context of the invention immunospecifically bind to polypeptides comprising or alternatively, consisting of, the amino acid sequence of SEQ ID NO:2, encoded by the cDNA contained in the plasmid having ATCC™ accession number 97768, or encoded by nucleic acids which hybridize (e.g., under stringent hybridization conditions) to the nucleotide sequence contained in the deposited clone. Antagonistic antibodies useful in the context of the invention also bind to fragments of the amino acid sequence of SEQ ID NO:2, encoded by the cDNA contained in the plasmid having ATCC™ accession number 97768, or encoded by nucleic acids which hybridize (e.g., under stringent hybridization conditions) to the nucleotide sequence contained in the deposited clone.

Additionally, antagonistic antibodies useful in the context of the invention bind polypeptides comprising or alternatively, consisting of, the amino acid sequence of SEQ ID NO:19, encoded by the cDNA contained in the plasmid having ATCC™ accession number 203518, or encoded by nucleic acids which hybridize (e.g., under stringent hybridization conditions) to the nucleotide sequence contained in the deposited clone. Antagonistic antibodies useful in the context of the invention also bind to fragments of the amino acid sequence of SEQ ID NO:19, encoded by the cDNA contained in the plasmid having ATCC™ accession number 203518, or encoded by nucleic acids which hybridize (e.g., under stringent hybridization conditions) to the nucleotide sequence contained in the deposited clone.

In specific embodiments, the antagonistic antibodies useful in the context of the invention immunospecifically bind polypeptide fragments including polypeptides comprising or alternatively, consisting of, an amino acid sequence contained in SEQ ID NO:2, encoded by the cDNA contained in the deposited clone, or encoded by nucleic acids which hybridize (e.g., under stringent hybridization conditions) to the nucleotide sequence contained in the deposited clone. Protein fragments may be “free-standing,” or comprised within a larger polypeptide of which the fragment forms a part or region, most preferably as a single continuous region. Representative examples of polypeptide fragments that may be bound by the antagonistic antibodies useful in the context of the invention, include, for example, fragments that comprise, or alternatively consist of, from about amino acid residues: 1-50, 51-100, 101-150, 151-200, 201-250, and/or 251-285 of SEQ ID NO:2. Moreover, polypeptide fragments can be at least 10, 20, 30, 40, 50, 60, 70, 80, 90, 100, 110, 120, 130, 140, 150, 175 or 200 amino acids in length.

In specific embodiments, antagonistic antibodies useful in the context of the invention bind polypeptide fragments comprising, or alternatively consisting of, amino acid residues: 1-46, 31-44, 47-72, 73-285, 73-83, 94-102, 148-152, 166-181, 185-209, 210-221, 226-237, 244-249, 253-265, and/or 277-285 of SEQ ID NO:2. In a specific embodiment, antagonistic antibodies useful in the context of the invention bind an epitope comprising amino acids 165-171 of SEQ ID NO:2.

Antagonistic antibodies useful in the context of the invention bind polypeptide fragments comprising, or alternatively consisting of, the intracellular domain of B Lymphocyte Stimulator protein (e.g., amino acid residues 1-46 of SEQ ID NO:2), the transmembrane domain of B Lymphocyte Stimulator protein (e.g., amino acid residues 47-72 of SEQ ID NO:2), the extracellular domain of B Lymphocyte Stimulator protein (e.g., amino acid residues 73-285 of SEQ ID NO:2), the mature soluble extracellular domain of B Lymphocyte Stimulator protein (e.g., amino acids residues 134-285 of SEQ ID NO:2), the TNF conserved domain of B Lymphocyte Stimulator protein (e.g., amino acids 191-284 of SEQ ID NO:2), and a polypeptide comprising, or alternatively, consisting of the intracellular domain fused to the extracellular domain of B Lymphocyte Stimulator protein (amino acid residues 1-46 fused to amino acid residues 73-285 of SEQ ID NO:2).

Antagonistic antibodies useful in the context of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. Immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule. In preferred embodiments, the immunoglobulin is an IgG1 or an IgG4 isotype. Immunoglobulins may have both a heavy and light chain. An array of IgG, IgE, IgM, IgD, IgA, and IgY heavy chains may be paired with a light chain of the kappa or lambda forms.

Most preferably, the antagonist antibodies useful in the context of the invention are human antigen-binding antibody fragments and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Antigen-binding fragments also comprise any combination of variable region(s) with a hinge region, CH1, CH2, and CH3 domains. The antagonistic antibodies useful in the context of the invention may be from any animal origin including birds and mammals. Preferably, the antibodies are human, murine (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example, in U.S. Pat. No. 5,939,598 by Kucherlapati et al.

The antagonistic antibodies useful in the context of the invention may be monospecific, bispecific, trispecific or of greater multispecificity. Multispecific antibodies may be specific for different epitopes of a polypeptide of the invention or may be specific for both B Lymphocyte Stimulator protein as well as for a heterologous epitope, such as a heterologous polypeptide or solid support material. See, e.g., International Patent Application Publications WO 93/17715; WO 92/08802; WO91/00360; and WO 92/05793; Tuft, et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Anti-B Lymphocyte Stimulator antibodies may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

Anti-B Lymphocyte Stimulator antibodies may be administered alone or in combination with other types of treatments (e.g., radiation therapy, chemotherapy, hormonal therapy, immunotherapy, anti-tumor agents, antibiotics, and immunoglobulins). Generally, administration of products of a species origin or species reactivity (in the case of antibodies) that is the same species as that of the patient is preferred. Thus, in a preferred embodiment, human antibodies, fragments, derivatives, analogs, or nucleic acids are administered to a human patient. In another embodiment, chimeric, humanized, or non-human monoclonal antibodies are administered to a human patient.

The antibodies useful in the context of the invention may be generated by any suitable method known in the art. Polyclonal antibodies can be produced by various procedures well known in the art. For example, a polypeptide can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

A “monoclonal antibody” may comprise, or alternatively consist of, two proteins, i.e., a heavy and a light chain.

Methods for producing and screening for specific antibodies using hybridoma technology are routine and well known in the art. In a non-limiting example, mice can be immunized with a polypeptide or a cell expressing a polypeptide. Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC™. Hybridomas are selected and cloned by limited dilution. The hybridoma clones are then assayed by methods known in the art for cells that secrete antibodies capable of binding the polypeptide. Ascites fluid, which generally contains high levels of antibodies, can be generated by immunizing mice with positive hybridoma clones.

Antibody fragments which recognize specific epitopes may be generated by known techniques. For example, Fab and F(ab′)2 fragments may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

For example, the antagonistic antibodies useful in the context of the invention can also be generated using various phage display methods known in the art. In phage display methods, functional antibody domains are displayed on the surface of phage particles which carry the polynucleotide sequences encoding them. In a particular embodiment, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); International Patent Application Publication WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in International Patent Application Publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entirety. Humanized antibodies are antibody molecules from non-human species antibody that binds the desired antigen having one or more complementarity determining regions (CDRs) from the non-human species and a framework region from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 0239400; International Patent Application Publication WO 91/09967; U.S. Pat. Nos. 5,225,539; 5,530,101; and 5,585,089), veneering or resurfacing (EP 0592106; EP 0519596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

Using phage display technology, single chain antibody molecules (“scFvs”) that immunospecifically bind to B Lymphocyte Stimulator have been identified, as described in U.S. Pat. No. 7,220,840, which is incorporated by reference herein, including scFvs that immunospecifically bind to soluble B Lymphocyte Stimulator, scFvs that immunospecifically bind the membrane-bound form of B Lymphocyte Stimulator, and scFvs that immunospecifically bind to both the soluble form and the membrane-bound form of B Lymphocyte Stimulator. Molecules comprising, or alternatively consisting of, fragments or variants of the scFvs described in U.S. Pat. No. 7,220,840 (e.g., including VH domains, VH CDRs, VL domains, or VL CDRs having an amino acid sequence of any one of those referred to in Table 1 of U.S. Pat. No. 7,220,840), that immunospecifically bind the soluble form of B Lymphocyte Stimulator, the membrane-bound form of B Lymphocyte Stimulator, and/or both the soluble form and membrane-bound form of B Lymphocyte Stimulator, are also encompassed by the invention, as are nucleic acid molecules that encode these scFvs, and/or molecules.

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and International Patent Application Publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., International Patent Application Publications WO 98/24893; WO 92/01047; WO 96/34096; WO 96/33735; European Patent 0598877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach, a selected non-human monoclonal antibody, e.g., a mouse antibody, is used to guide the selection of a completely human antibody recognizing the same epitope. (Jespers et al., Bio/technology 12:899-903 (1988)).

In a preferred embodiment, antibodies useful in the context of the invention immunospecifically bind to the soluble form of B Lymphocyte Stimulator and comprise, or alternatively consist of, a VH domain, VH CDR1, VH CDR2, VH CDR3, VL domain, VL CDR1, VL CDR2, and/or VL CDR3 corresponding to one or more scFvs described in U.S. Patent Application Publication 2005/0255532, that immunospecifically bind to the soluble form of B Lymphocyte Stimulator. In another preferred embodiment, antibodies useful in the context of the invention immunospecifically bind to the membrane-bound form of B Lymphocyte Stimulator and comprise, or alternatively consist of, a VH domain, VH CDR1, VH CDR2, VH CDR3, VL domain, VL CDR1, VL CDR2, and/or VL CDR3 corresponding to one or more scFvs described in U.S. Patent Application Publication 2005/0255532, that immunospecifically bind to the membrane-bound form of B Lymphocyte Stimulator. In yet another preferred embodiment, antibodies useful in the context of the invention immunospecifically bind to the soluble form and membrane-bound form of B Lymphocyte Stimulator and comprise, or alternatively consist of, a VH domain, VH CDR1, VH CDR2, VH CDR3, VL domain, VL CDR1, VL CDR2, and/or VL CDR3 corresponding to one or more scFvs described in U.S. Patent Application Publication 2005/0255532, that immunospecifically binds to the soluble form and membrane-bound form of B Lymphocyte Stimulator. Nucleic acid molecules encoding these antibodies are also encompassed by the invention.

The invention also provides antibodies (including molecules comprising or alternatively consisting of, antibody fragments or variants thereof) that immunospecifically bind to a heterotrimeric protein comprising at least one B Lymphocyte Stimulator polypeptide (preferably amino acids 134-285 of SEQ ID NO:2), said antibodies comprising, or alternatively consisting of, a polypeptide having the amino acid sequence of any one of the VH domains and any one of the VL domains referred to in U.S. Pat. No. 7,220,840. Molecules comprising, or alternatively consisting of, fragments or variants of these antibodies (e.g., including VH domains, VH CDRs, VL domains, or VL CDRs having an amino acid sequence of any one of those referred to in U.S. Pat. No. 7,220,840), that immunospecifically bind a heterotrimeric protein comprising at least one B Lymphocyte Stimulator polypeptide, are also encompassed by the invention, as are nucleic acid molecules that encode these antibodies, and/or molecules.

The invention provides antibodies (including molecules comprising, or alternatively consisting of, antibody fragments or variants thereof (including derivatives)) that immunospecifically bind to B Lymphocyte Stimulator (e.g., soluble B Lymphocyte Stimulator and membrane-bound B Lymphocyte Stimulator) and can be routinely assayed for immunospecific binding to B Lymphocyte Stimulator using methods known in the art. Antibodies and antibody fragments or variants (including derivatives) of the invention may include, for example, one or more amino acid sequence alterations (addition, deletion, substitution and/or insertion of an amino acid residue). These alterations may be made in one or more framework regions and/or one or more CDR's. The antibodies of the invention (including antibody fragments, and variants and derivative thereof) can be routinely made by methods known in the art. Molecules comprising, or alternatively consisting of, fragments or variants of any of the VH domains, VH CDRs, VL domains, and VL CDRs whose sequences are specifically disclosed herein may be employed in accordance with the invention. Nucleic acid molecules encoding these antibodies and molecules (including fragments, variants, and derivatives) are also encompassed by the invention.

In specific embodiments, the invention encompasses a single chain Fv (scFv) having an amino acid sequence of SEQ ID NO: 60 or SEQ ID NO: 61.

In specific embodiments, the invention encompasses an antibody or fragment thereof comprising a VH domain from an scFv having an amino acid sequence of SEQ ID NO: 60 or SEQ ID NO: 61, wherein said antibody or fragment thereof immunospecifically binds B Lymphocyte Stimulator.

In specific embodiments, the invention encompasses an antibody or fragment thereof comprising a VL domain from an scFv having an amino acid sequence of SEQ ID NO: 60 or SEQ ID NO: 61, wherein said antibody or fragment thereof immunospecifically binds B Lymphocyte Stimulator.

In specific embodiments, the invention encompasses an antibody or fragment thereof comprising a VL domain from an scFv having an amino acid sequence of SEQ ID NO: 60 or SEQ ID NO: 61, wherein said antibody or fragment thereof immunospecifically binds B Lymphocyte Stimulator and which also comprises a VH domain from an scFv having an amino acid sequence of SEQ ID NO: 60 or SEQ ID NO: 61.

In specific embodiments, the invention encompasses an antibody or fragment thereof comprising a VL domain from an scFv having an amino acid sequence of SEQ ID NO: 60, wherein said antibody or fragment thereof immunospecifically binds B Lymphocyte Stimulator and which also comprises a VH domain from an scFv having an amino acid sequence of SEQ ID NO: 60.

In specific embodiments, the invention encompasses an antibody or fragment thereof comprising a VL domain from an scFv having an amino acid sequence of SEQ ID NO: 61, wherein said antibody or fragment thereof immunospecifically binds B Lymphocyte Stimulator and which also comprises a VH domain from an scFv having an amino acid sequence of SEQ ID NO: 61.

In specific embodiments, the antibody or fragment thereof of the invention is a whole immunoglobulin molecule.

In specific embodiments, the antibody or fragment thereof of the invention is a Fab fragment.

In specific embodiments, the antibody or fragment thereof of the invention is a Fv fragment.

In specific embodiments, the invention encompasses a chimeric protein comprising the antibody or fragment thereof of the invention covalently linked to a heterologous polypeptide.

In specific embodiments, the invention encompasses a composition comprising two or more types of antibodies or fragments or variants thereof, each of which type immunospecifically binds to B Lymphocyte Stimulator, and each of which type of antibody or fragment thereof comprises a VH domain from a different scFv having an amino acid sequence of SEQ ID NO: 60 or SEQ ID NO: 61.

In specific embodiments, the invention encompasses a composition comprising two or more types of antibodies or fragments or variants thereof, each of which type immunospecifically binds to B Lymphocyte Stimulator, and each of which type of antibody or fragment thereof comprises a VL domain from a different scFv having an amino acid sequence of SEQ ID NO: 60 or SEQ ID NO: 61.

In specific embodiments, the invention encompasses a composition comprising two or more types of antibodies or fragments or variants thereof, each of which type immunospecifically binds to B Lymphocyte Stimulator, and each of which type of antibody or fragment thereof comprises a VL domain from a different scFv having an amino acid sequence of one SEQ ID NO: 60 or SEQ ID NO: 61 and wherein each type of antibody or fragment thereof further comprises a VH domain from a different scFv having an amino acid sequence of SEQ ID NO: 60 or SEQ ID NO: 61.

In specific embodiments, the invention encompasses a panel of two or more types of antibodies or fragments or variants thereof, each of which type immunospecifically binds to B Lymphocyte Stimulator, and each of which type of antibody or fragment thereof comprises a VH domain from a different scFv having an amino acid sequence of SEQ ID NO: 60 or SEQ ID NO: 61.

In specific embodiments, the invention encompasses a panel of two or more types of antibodies or fragments or variants thereof, each of which type immunospecifically binds to B Lymphocyte Stimulator, and each of which type of antibody or fragment thereof comprises a VL domain from a different scFv having an amino acid sequence of SEQ ID NO: 60 or SEQ ID NO: 61.

In specific embodiments, the invention encompasses a panel of two or more types of antibodies or fragments or variants thereof, each of which type immunospecifically binds to B Lymphocyte Stimulator, and each of which type of antibody or fragment thereof comprises a VL domain from a different scFv having an amino acid sequence of SEQ ID NO: 60 or SEQ ID NO: 61 and wherein each type of antibody or fragment further comprises a VH domain from a different scFv having an amino acid sequence of SEQ ID NO: 60 or SEQ ID NO: 61.

The VL domain of SEQ ID NO: 60 comprises amino acid residues 1-123 of SEQ ID NO: 60 and the VH domain of SEQ ID NO: 60 comprises amino acid residues 141-249 of SEQ ID NO: 60. Thus, in one embodiment, the invention encompasses an anti-BLyS antibody comprising a first amino acid sequence comprising amino acid residues 1-123 of SEQ ID NO: 60 and a second amino acid sequence comprising amino acid residues 141-249 of SEQ ID NO: 60.

The VL domain of SEQ ID NO: 61 comprises amino acid residues 1-123 of SEQ ID NO: 61 and the VH domain of SEQ ID NO: 61 comprises amino acid residues 141-249 of SEQ ID NO: 61. Thus, in one embodiment, the invention encompasses an anti-BLyS antibody comprising a first amino acid sequence comprising amino acid residues 1-123 of SEQ ID NO: 61 and a second amino acid sequence comprising amino acid residues 141-249 of SEQ ID NO: 61.

The VLCDR1, VLCDR2, and VLCDR3 regions of SEQ ID NO: 60 comprise amino acid residues 163-173, 189-195, and 228-238, respectively. The VHCDR1, VHCDR2, and VHCDR3 regions of SEQ ID NO: 60 comprise amino acid residues 26-35, 50-66, and 99-112, respectively. Thus, in one embodiment, the invention encompasses an anti-BLyS antibody comprising amino acid residues 26-35, 50-66, 99-112, 163-173, 189-195, and 228-238 of SEQ ID NO: 60.

The VLCDR1, VLCDR2, and VLCDR3 regions of SEQ ID NO: 61 comprise amino acid residues 163-173, 189-195, and 228-238, respectively. The VHCDR1, VHCDR2, and VHCDR3 regions of SEQ ID NO: 61 comprise amino acid residues 26-35, 50-66, and 99-112, respectively. Thus, in one embodiment, the invention encompasses an anti-BLyS antibody comprising amino acid residues 26-35, 50-66, 99-112, 163-173, 189-195, and 228-238 of SEQ ID NO: 61.

In specific embodiments, the invention encompasses the antibody BENLYSTA® (belimumab) from Human Genome Sciences, Inc.

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of SEQ ID NO:2. In another preferred embodiment, the antibody binds specifically to a polypeptide having the amino acid sequence of SEQ ID NO:19. In another preferred embodiment, the antibody binds specifically to a polypeptide having the amino acid sequence of SEQ ID NO:23. In another preferred embodiment, the antibody binds specifically to a polypeptide having the amino acid sequence of SEQ ID NO:28. In another preferred embodiment, the antibody binds specifically to a polypeptide having the amino acid sequence of SEQ ID NO:30. In another preferred embodiment, the antibody binds specifically to a polypeptide having the amino acid sequence of SEQ ID NO:39. In another preferred embodiment, the antibody binds specifically to a polypeptide having the amino acid sequence of SEQ ID NO:40. In another embodiment, the antibody binds specifically to a polypeptide having the amino acid sequence of SEQ ID NO:41. In another embodiment, the antibody binds specifically to a polypeptide having the amino acid sequence of SEQ ID NO:42. In another embodiment, the antibody binds specifically to a polypeptide having the amino acid sequence of SEQ ID NO:43. In another embodiment, the antibody binds specifically to a polypeptide having the amino acid sequence of SEQ ID NO:44.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example, to create amino acid substitutions, deletions, and/or insertions.

In a specific embodiment, the amino acid sequence of the heavy and/or light chain variable domains may be inspected to identify the sequences of the complementarity determining regions (CDRs) by methods that are well known in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

### Methods of Producing Antibodies

The antibodies of the invention can be produced by any method known in the art for the synthesis of antibodies, in particular, by chemical synthesis or preferably, by recombinant expression techniques.

Recombinant expression of an antibody of the invention, or fragment, derivative or analog thereof (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., International Patent Application Publications WO 86/05807 and WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO) in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (E.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5):155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the invention or fragments thereof can be fused to heterologous polypeptide sequences described herein or otherwise known in the art, to facilitate purification.

The invention encompasses antibodies recombinantly fused or chemically conjugated (including both covalent and non-covalent conjugations) to a polypeptide (or portion thereof, preferably at least 10, 20, 30, 40, 50, 60, 70, 80, 90 or 100 amino acids of the polypeptide) of the invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the invention. For example, antibodies may be used to target the polypeptides of the invention to particular cell types, either in vitro or in vivo, by fusing or conjugating the polypeptides of the invention to antibodies specific for particular cell surface receptors. Antibodies fused or conjugated to the polypeptides of the invention may also be used in in vitro immunoassays and purification methods using methods known in the art. See e.g., Harbor et al., supra, and International Patent Application Publication WO 93/21232; EP 0439095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452 (1991), which are incorporated by reference in their entireties.

The invention further includes compositions comprising the polypeptides of the invention fused or conjugated to antibody domains other than the variable regions. For example, the polypeptides of the invention may be fused or conjugated to an antibody Fc region, or portion thereof. The antibody portion fused to a polypeptide of the invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the invention can form dimers through disulfide bonding between the Fc portions. Higher multimeric forms can be made by fusing the polypeptides to portions of IgA and IgM. Methods for fusing or conjugating the polypeptides of the invention to antibody portions are known in the art. See, e.g., U.S. Pat. Nos. 5,336,603; 5,622,929; 5,359,046; 5,349,053; 5,447,851; and 5,112,946; EP 0307434; EP 0367166; International Patent Application Publications WO 96/04388 and WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341 (1992) (said references incorporated by reference in their entireties).

As discussed, supra, the polypeptides corresponding to a polypeptide, polypeptide fragment, or a variant of SEQ ID NO:2 may be fused or conjugated to the above antibody portions to increase the in vivo half life of the polypeptides or for use in immunoassays using methods known in the art. Further, the polypeptides corresponding to SEQ ID NO:2 may be fused or conjugated to the above antibody portions to facilitate purification. Also as discussed, supra, the polypeptides corresponding to a polypeptide, polypeptide fragment, or a variant of SEQ ID NO:19 may be fused or conjugated to the above antibody portions to increase the in vivo half life of the polypeptides or for use in immunoassays using methods known in the art. Moreover, the polypeptides corresponding to SEQ ID NO:19 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins (EP 0394827; Traunecker et al., Nature 331:84-86 (1988). The polypeptides of the invention fused or conjugated to an antibody having disulfide-linked dimeric structures (due to the IgG) may also be more efficient in binding and neutralizing other molecules than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties (EP 0232262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5 (See, Bennett et al., J. Molecular Recognition 8:52-58 (1995); Johanson et al., J. Biol. Chem. 270:9459-9471 (1995).

Moreover, the antibodies or fragments thereof of the invention can be fused to marker sequences, such as a peptide, to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include iodine (¹³¹I, ¹²⁵I, ¹²³I, ¹²¹I), carbon (¹⁴C), sulfur (³⁵S), tritium (³H), indium (¹¹⁵mIn, ¹¹³mIn, ¹¹²_{In,} ¹¹¹In), and technetium (⁹⁹Tc, ⁹⁹ mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Se, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin.

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, ²¹³Bi. In specific embodiments, antibodies of the invention are attached to macrocyclic chelators useful for conjugating radiometal ions, including but not limited to, ¹¹¹In, ¹⁷⁷Lu, ⁹⁰Y, ¹⁶⁶Ho, and ¹⁵³Sm, to polypeptides. In preferred embodiments, the radiometal ion associated with the macrocyclic chelators attached to antibodies of the invention is ¹¹¹In. In preferred embodiments, the radiometal ion associated with the macrocyclic chelators attached to antibodies of the invention is ⁹⁰Y. In specific embodiments, the macrocyclic chelator is 1,4,7,10-tetraazacyclododecane-N,N′,N″,N″′-tetraacetic acid (DOTA). In other specific embodiments, the DOTA is attached to the B Lymphocyte Stimulator polypeptide of the invention via a linker molecule. Examples of linker molecules useful for conjugating DOTA to a polypeptide are commonly known in the art—see, for example, DeNardo et al., Clin Cancer Res. 4(10):2483-90 (1998); Peterson et al., Bioconjug. Chem. 10(4):553-7 (1999); and Zimmerman et al, Nucl. Med. Biol. 26(8):943-50 (1999) which are hereby incorporated by reference in their entirety. In addition, U.S. Pat. Nos. 5,652,361 and 5,756,065, which disclose chelating agents that may be conjugated to antibodies and methods for making and using them, are hereby incorporated by reference in their entireties.

A cytotoxin or cytotoxic agent includes any agent that is detrimental to cells and includes such molecules as small molecule toxins and enzymatically active toxins of bacterial, fungal, plant, or animal origin, or fragments thereof. Examples include paclitaxol, cytochalasin B, gramicidin D, ethidium bromide, emetine, mitomycin, etoposide (VP-16), tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine), improsulfan, piposulfan, benzodopa, carboquone, meturedopa, uredopa, altretamine, triethylenemelamine, trietylenephosphoramide, triethylenethiophosphaoramide, trimethylolomelamine, chlornaphazine, cholophosphamide, estramustine, ifosfamide, novembichin, phenesterine, prednimustine, trofosfamide, uracil mustard, chlorozotocin, fotemustine, nimustine, ranimustine, aclacinomysins, azaserine, cactinomycin, calichearnicin, carabicin, caminomycin, carzinophilin, chromomycins, detorubicin, 6-diazo-5-oxo-L-norleucine, epirubicin, esorubicin, idarubicin, marcellomycin, mycophenolic acid, nogalamycin, olivomycins, peplomycin, potfiromycin, quelamycin, rodorubicin, streptonigrin, tubercidin, ubenimex, zinostatin, zorubicin, denopterin, pteropterin, trimetrexate, fludarabine, thiamiprine, ancitabine, azacitidine, 6-azauridine, carmofur, dideoxyuridine, doxifluridine, enocitabine, floxuridine, 5-FU, calusterone, dromostanolone propionate, epitiostanol, mepitiostane, testolactone, aminoglutethimide, mitotane, trilostane, frolinic acid, aceglatone, aldophosphamide glycoside, aminol evulinic acid, amsacrine, bestrabucil, bisantrene, edatraxate, defofamine, dernecolcine, diaziquone, elfornithine, elliptiniurn acetate, etoglucid, gallium nitrate, hydroxyurea, lentinan, lonidamine, mitoguazone, mopidamol, nitracrine, pentostatin, phenamet, pirarubicin, podophyllinic acid, 2-ethylhydrazide, procarbazine, PSKO, razoxane, sizofuran, spirogermanium, tenuazonic acid, triaziquone, 2, 2′,2″-trichlorotriethylamine, urethan, vindesine, dacarbazine, mannomustine, mitobronitol, mitolactol, pipobroman, gacytosine, arabinoside (“Ara-C”), taxoids, e.g. paclitaxel (TAXOL®, Bristol-Myers Squibb Oncology, Princeton, N.J.) doxetaxel (TAXOTERE®, Rh6ne-Poulenc Rorer, Antony, France), gemcitabine, ifosfamide, vinorelbine, navelbine, novantrone, teniposide, aminopterin, xeloda, ibandronate, CPT-I 1, topoisomerase inhibitor RFS 2000, difluoromethylornithine (DMFO), retinoic acid, esperamicins, capecitabine, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4 hydroxytamoxifen, trioxifene, keoxifene, LY 117018, onapristone, toremifene (Fareston), and anti-androgens such as flutamide, nilutamide, bicalutamide, leuprolide, and goserelin, and pharmaceutically acceptable salts, acids or derivatives of any of the above.

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I (see International Patent Application Publication WO 97/33899), AIM II (see International Patent Application Publication WO 97/34911), Fas Ligand (Takahashi et al., Int. Immunol., 6:1567-1574 (1994)), VEGI (see International Patent Application Publication WO 99/23105), CD40 Ligand, a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody in Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

### Assays For Antibody Binding

The antibodies of the invention may be assayed for immunospecific binding by any method known in the art. The immunoassays which can be used include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

### Demonstration of Therapeutic or Prophylactic Activity

The B Lymphocyte Stimulator antagonists or pharmaceutical compositions thereof preferably are tested in vitro, and then in vivo, for the desired therapeutic or prophylactic activity, prior to use in humans. For example, in vitro assays to demonstrate the therapeutic or prophylactic utility of a B Lymphocyte Stimulator antagonist or pharmaceutical composition thereof include evaluating the effectiveness on a cell line or a patient tissue sample. The effect on the cell line and/or tissue sample can be determined utilizing techniques known to those of skill in the art including, but not limited to, rosette formation assays and cell lysis assays. In vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

### EXAMPLES

Having generally described the invention, the same will be more readily understood by reference to the following examples, which are provided by way of illustration and are not intended as limiting. One of ordinary skill in the art would easily be able to direct the following examples.

### Example 1

This example demonstrates the isolation of antibody fragments directed against B Lymphocyte Stimulator protein from a library of scFvs, as previously described in U.S. Pat. No. 7,220,840 which is herein incorporated by reference.

Naturally occurring V-genes isolated from human PBLs are constructed into a large library of antibody fragments which contain reactivities against B Lymphocyte Stimulator protein to which the donor may or may not have been exposed (see e.g., U.S. Pat. No. 5,885,793 incorporated herein in its entirety by reference).

### Rescue of the Library

A library of scFvs is constructed from the RNA of human PBLs as described in WO92/01047 (which is hereby incorporated by reference in its entirety). To rescue phage displaying antibody fragments, approximately 10⁹ E. coli harboring the phagemid are used to inoculate 50 ml of 2×TY containing 1% glucose and 100 micrograms/ml of ampicillin (2×TY-AMP-GLU) and grown to an O.D. of 0.8 with shaking Five ml of this culture is used to inoculate 50 ml of 2×TY-AMP-GLU, 2× 10⁸ TU of delta gene 3 helper (M13 delta gene III, see WO92/01047) are added and the culture incubated at 37° C. for 45 minutes without shaking and then at 37° C. for 45 minutes with shaking The culture is centrifuged at 4000 r.p.m. for 10 min. and the pellet resuspended in 2 liters of 2×TY containing 100 micrograms/ml ampicillin and 50 micrograms/ml kanamycin and grown overnight. Phage are prepared as described in WO92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking Cells were spun down (IEC-Centra 8, 4000 revs/min for 10 min), resuspended in 300 ml 2×TY broth containing 100 micrograms ampicillin/ml and 25 micrograms kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 micrometer filter (Minisart NML; Sartorius) to give a final concentration of approximately 10¹³ transducing units/ml (ampicillin-resistant clones).

### Panning the Library

Immunotubes (Nunc) are coated overnight in PBS with 4 ml of either 100 micrograms/ml or 10 micrograms/ml of a polypeptide of the invention. Tubes are blocked with 2% Marvel-PBS for 2 hours at 37° C. and then washed 3 times in PBS. Approximately 10¹³ TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 micrograms/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

### Characterization of Binders

Eluted phage from the third and fourth rounds of selection are used to infect E. coli HB 2151 and soluble scFv is produced (Marks, et al., 1991) from single colonies for assay. ELISAs are performed with microtiter plates coated with either 10 picograms/ml of the polypeptide of the invention in 50 mM bicarbonate pH 9.6. Clones positive in ELISA are further characterized by PCR fingerprinting (see e.g., WO92/01047) and then by sequencing.

### Example 2

This example demonstrates the neutralization of B Lymphocyte Stimulator protein receptor interaction with an anti-B Lymphocyte Stimulator protein monoclonal antibody, as previously described U.S. Pat. No. 6,881,401 and U.S. Patent Application Publication 2009/0104189 A1, which are incorporated by reference herein.

Monoclonal antibodies were generated against B Lymphocyte Stimulator protein according to the following method. Briefly, mice were given a subcutaneous injection (front part of the dorsum) of 50 micrograms of His-tagged B Lymphocyte Stimulator protein in 100 microliters of PBS emulsified in 100 microliters of complete Freunds adjuvant. Three additional subcutaneous injections of 25 micrograms of B Lymphocyte Stimulator protein in incomplete Freunds adjuvant were given at 2-week intervals. The animals were rested for a month before they received the final intraperitoneal boost of 25 micrograms of B Lymphocyte Stimulator protein in PBS. Four days later mice were sacrificed and splenocytes taken for fusion.

The process of “Fusion” was accomplished by fusing splenocytes from one spleen with 2× 10⁷ P3X63Ag8.653 plasmacytoma cells using PEG 1500 (Boehringer Mannheim), according to the manufacturer's modifications of an earlier described method. (See, Gefter, M. L., et al. Somatic Cell Genet. 3:231-36 (1977); Boehringer Mannheim, PEG 1500 (Cat.No. 783641), product description.)

After fusion, the cells were resuspended in 400 ml of HAT medium supplemented with 20% FBS and 4% Hybridoma Supplement (Boehringer Mannheim) and distributed to 96 well plates at a density of 200 microliters per well. At day 7 post-fusion, 100 microliters of medium was aspirated and replaced with 100 microliters of fresh medium. At day 14 post-fusion, the hybridomas were screened for antibody production.

Hybridoma supernatants were screened by ELISA for binding to B Lymphocyte Stimulator protein immobilized on plates. Plates were coated with B Lymphocyte Stimulator protein by overnight incubation of 100 microliters per well of B Lymphocyte Stimulator protein in PBS at a concentration of 2 micrograms per ml. Hybridoma supernatants were diluted 1:10 with PBS were placed in individual wells of B Lymphocyte Stimulator protein-coated plates and incubated overnight at 4° C. On the following day, the plates were washed 3 times with PBS containing 0.1% Tween-20 and developed using the anti-mouse IgG ABC system (Vector Laboratories). The color development reaction was stopped with the addition of 25 ml/well of 2M H₂SO₄. The plates were then read at 450 nm.

Hybridoma supernatants were checked for Ig isotype using Isostrips. Cloning was done by the method of limiting dilutions on HT medium. About 3×10⁶ cells in 0.9 ml of HBSS were injected in pristane-primed mice. After 7-9 days, ascitic fluid was collected using a 19 g needle. All antibodies were purified by protein G affinity chromatography using the Acta FPLC system (Pharmacia).

After primary and two consecutive subcutaneous injections, all three mice developed a strong immune response; the serum titer was 10⁻⁷ as assessed by ELISA on B Lymphocyte Stimulator protein-coated plates.

In one experiment, using the splenocytes from the positive mouse more than 1000 primary hybridomas were generated. 917 of them were screened for producing anti-B Lymphocyte Stimulator protein antibody. Screening was performed using 1:1 diluted supernatants in order to detect all positive clones. Of 917 hybridomas screened, 76 were found to be positive and 17 of those were found to be IgG producers. After affinity testing and cloning, 9 of them were chosen for further expansion and purification.

All purified monoclonal antibodies were able to bind different forms of B Lymphocyte Stimulator protein (including His-tagged and protein produced from a baculoviral system in both Western blot analysis and ELISA. Six of nine clones were also able to bind B Lymphocyte Stimulator protein on the surface of THP-1 cells. However, none of the antibodies tested were able to capture B Lymphocyte Stimulator protein from solution.

High affinity anti-B Lymphocyte Stimulator monoclonal antibodies were generated that recognize B Lymphocyte Stimulator protein expressed on the cell surface but not in solution can be used for neutralization studies in vivo and in monocyte and B cell assays in vitro. These antibodies are also useful for sensitive detection of B Lymphocyte Stimulator protein on Western blots.

In an independent experiment, using the splenocytes from the positive mouse, more than 1000 primary hybridomas were generated. 729 of the primary hybridomas were then screened for the production of an anti-B Lymphocyte Stimulator protein antibody. Screening was performed under stringent conditions using 1:10 diluted supernatants in order to pick up only clones of higher affinity. Of 729 hybridomas screened, 23 were positive, including 16 IgM and 7 IgG producers (among the latter, 4 gave a strong IgM background). In this experiment, the isotype distribution of IgG antibodies was biased towards the IgG2 subclasses. Three of seven IgG hybridomas produced antibodies of IgG2a subclass and two produced an antibody of IgG2b subclass, while the remaining two were IgG1 producers.

Supernatants from all positive hybridomas generated in the second experiment were tested for the ability to inhibit B Lymphocyte Stimulator protein-mediated proliferation of B cells. In the first screening experiment, two hybridomas producing IgG-neutralizing antibodies were detected (these are antibodies 16C9 and 12C5). In additional experiments, the IgG-neutralizing activity of the hybridomas (i.e., 16C9 and 12C5) were confirmed and two additional strongly neutralizing supernatants from hybridomas 15C10 and 4A6 were identified.

Three clones were subsequently expanded in vivo (a single clone, i.e., 15C10, was also expanded in a hollow fiber system), and the antibody purified by affinity chromatography. All three of the clones were able to bind B Lymphocyte Stimulator protein on the surface of THP-1 cells and were also able to bind (i.e., “capture”) B Lymphocyte Stimulator protein from solution.

Specifically, experiments were performed using the anti-B Lymphocyte Stimulator protein monoclonal antibodies described in the second experiment above to determine whether the antibodies neutralize B Lymphocyte Stimulator protein/B Lymphocyte Stimulator protein receptor binding. Briefly, B Lymphocyte Stimulator protein was biotinylated using the EZ-link T NHS-biotin reagent (Pierce, Rockford, Ill.). Biotinylated B Lymphocyte Stimulator protein was then used to identify cell surface proteins that bind B Lymphocyte Stimulator protein. Preliminary experiments demonstrated that B Lymphocyte Stimulator protein binds to a receptor on B lymphoid cells.

The inclusion of anti-B Lymphocyte Stimulator protein antibodies generated in the second experiment described above neutralized binding of B Lymphocyte Stimulator protein to a B Lymphocyte Stimulator receptor. In a specific embodiment, anti-B Lymphocyte Stimulator antibody 15C10 neutralizes binding of B Lymphocyte Stimulator protein to a B Lymphocyte Stimulator Receptor.

Thus, the anti-B Lymphocyte Stimulator monoclonal antibodies generated in the second experiment described above (in particular, antibody 15C10) recognize and bind to both membrane-bound and soluble B Lymphocyte Stimulator protein and neutralize B Lymphocyte Stimulator protein/B Lymphocyte Stimulator Receptor binding in vitro.

### Example 3

This example demonstrates competitive binding studies between antibody 15C10 and 3D4.

To determine if antibodies 15C10 and 3D4 bind similar or distinct epitopes, competitive binding studies were performed.

Soluble B Lymphocyte Stimulator protein was preincubated with 15C10 or 3D4 antibodies. Hereinafter in this example, the antibody with which B Lymphocyte Stimulator protein was preincubated will be referred to as the “competing antibody”. After preincubation, soluble B Lymphocyte Stimulator protein-competing antibody complexes were captured on an ELISA plate coated with either 3D4 or 15C10. Hereinafter in this example, the antibody coated on the ELISA plate will be referred to as the “capture antibody.” After binding, and wash steps, soluble B Lymphocyte Stimulator protein-competing antibody complexes captured on the 3D4 or 15C10-coated ELISA plates was detected using a biotinylated polyclonal anti-B Lymphocyte Stimulator protein antibody followed by a streptavidin-coupled detection agent such as horse radish peroxidase or alkaline phosphatase.

If there is no competition between the competing antibody and the capture antibody on the ELISA plate (i.e., if the two antibodies bind non-overlapping epitopes), soluble B Lymphocyte Stimulator protein will be not prevented from binding to the capture antibody on the ELISA plate and the ELISA will give a positive signal. On the other hand, if there is competition between the competing antibody and the capture antibody on the ELISA plate (i.e., if the two antibodies bind overlapping or identical epitopes), a decreased (or no) amount of soluble B Lymphocyte Stimulator protein will be bound to the ELISA plate and the ELISA will give a decreased signal, compared to the signal given in the absence of competition between the two antibodies.

When an assay similar to that described above was performed using monoclonal antibodies 15C10 and 3D4, it was found that the two antibodies competed with each other, irrespective of which antibody was the competing antibody and which antibody was the capture antibody. These results indicate that 15C10 and 3D4 at least have overlapping epitopes. Isotype matched controls of irrelevant specificity (non-B Lymphocyte Stimulator protein binding) were not able to compete for binding.

### Example 4

This example demonstrates that in vivo B Lymphocyte Stimulator (BLyS) protein neutralization can prevent, ameliorate and/or treat allergen-induced allergic or inflammatory conditions of the lung by reducing total IgE levels, mucus accumulation and infiltration of cells in the lung. In particular, administration of a neutralizing monoclonal IgG1 hamster anti-mouse BLyS (clone 10F4) in a murine model of asthma prevented the onset of bronchial or lung inflammation when administered before or after exposure to an allergen known to induce lung inflammation.

BLyS is a key protein that drives the maturation and survival of B cells. A well-characterized murine model of allergic asthma, in which allergen exposure leads to airway hyper responsiveness, pulmonary eosinophilia, elevations in antigen-specific serum IgE levels, and increases in airway epithelial mucus content, was used to investigate the use of an anti-BLyS mAb as a potential therapeutic for the treatment and prevention of an allergic or inflammatory condition of the lung such as asthma.

### Ovalbumin Sensitization and Challenge

Female BALB/c mice (8-10 weeks of age) were sensitized with intraperitoneal (i.p.) injections of 20 μg (0.1 ml of 200 μg/ml in PBS) ovalbumin (OVA) (Sigma-Aldrich) every other day from day 1 through 19. Starting at day 33, the mice were challenged by nebulization with 10 mg/ml ovalbumin in PBS for 40 minutes every other day for four days, (i.e., the mice were challenged on days 33, 35, 37, and 39). The mice were terminated on day 40, after the last ovalbumin challenge. Control mice were either sensitized and challenged with PBS or left untreated (naïve) and terminated on day 40. Upon termination, blood, lung, bronchoalveolar lavage (BAL) fluid, and spleen were removed from the animals. The blood was centrifuged and the serum was removed.

### 10F4 and Dexamethasone Treatment in OVA-Sensitized Mice

To assess the ability of a B Lymphocyte Stimulator antagonist to prevent the onset of lung inflammation, one group of OVA-sensitized mice was treated with 100 μg of monoclonal IgG1 hamster anti-mouse BLyS (clone 10F4) by i.p. injection on days 20 and 27, prior to challenge with OVA (prophylactic group). 10F4 is a monoclonal IgG1 hamster anti-murine BLyS antibody that neutralizes murine BLyS as previously described in Schoz et al. Proc Natl Acad Sci USA. 2008 Oct. 7; 105(40): 15517-15522, which is herein incorporated by reference. As a control, one group of OVA-sensitized mice was treated with 100 μg of an isotype control antibody (purified Armenian hamster IgG1 (clone G235-2356; BD Biosciences)) by i.p. injection on days 20 and 27, prior to challenge with OVA (isotype control prophylactic (Iso Pro) group).

To assess the ability of a B Lymphocyte Stimulator antagonist to prevent, ameliorate or treat lung inflammation, one group of OVA-sensitized mice was treated with 100 μg of monoclonal IgG1 hamster anti-mouse BLyS (clone 10F4) by i.p. injection on day 34 (i.e., during OVA challenge) (therapeutic group). As a control, one group of OVA-sensitized mice was treated with 100 μg of an isotype control antibody (purified Armenian hamster IgG1 (clone G235-2356; BD Biosciences)) by i.p. injection on day 34 (isotype control therapeutic (Iso Ther or Iso Cont) group).

To compare the efficacy of 10F4 to classical treatment with glucocorticoids, one group of OVA-sensitized mice was given water soluble dexamethasone (DEX) (Sigma-Aldrich) (50 μg in 100 μl of PBS) by i.p. injection every other day of OVA challenge (i.e., dexamethasone was administered on days 34, 36, and 38).

The PBS and naïve groups of mice were left untreated.

All mice were terminated on day 40.

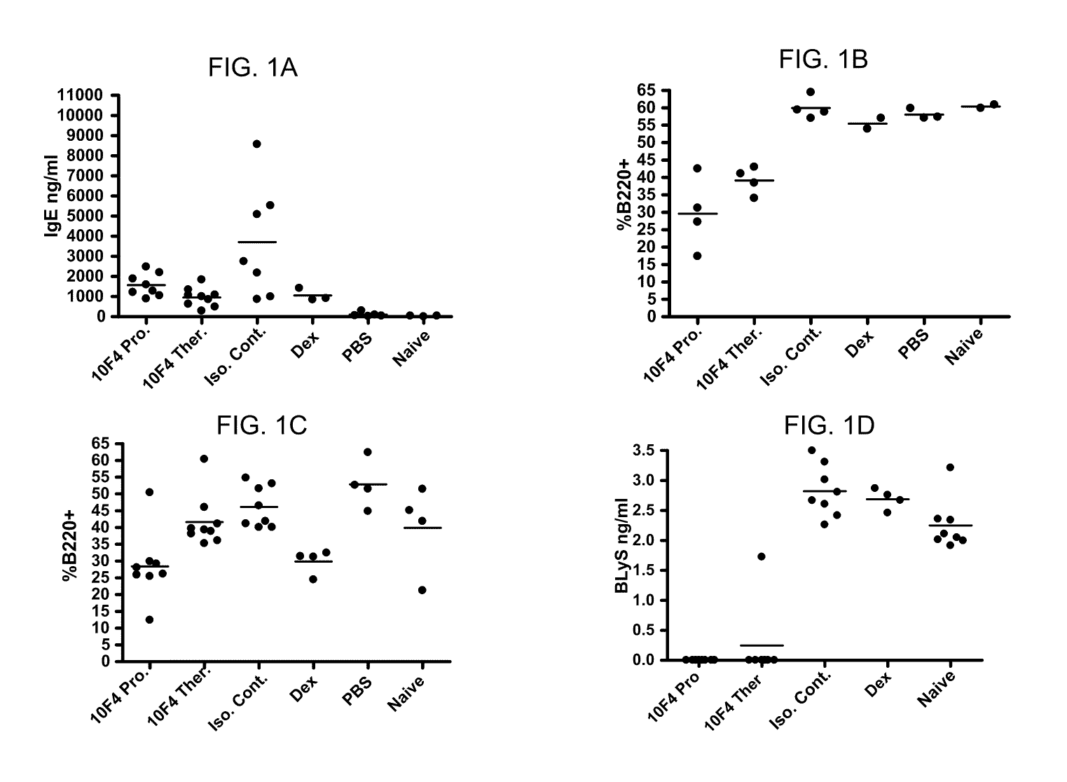

As shown in FIG. 1D, there was a significant reduction in serum BLyS levels in the prophylactic group (10F4 Pro) or the therapeutic group (10F4 Ther) as compared to mice treated with isotype control antibody (Iso. Cont.) or dexamethasone (Dex) and untreated (naïve) mice. These data confirm that treatment with anti-BLyS mAb 10F4 led to in vivo BLyS neutralization.

### Total IgE ELISA

Serum IgE was detected using BD Biosciences Mouse OptEIA Total IgE ELISA Kit. Briefly, plates were coated with Capture Antibody, blocked with 2% BSA in PBS, and serum dilutions were incubated for 2 hours. Detection antibody and Sav-HRP reagent was added and incubated for 1 hour. TMB substrate solution was added and allowed to incubate for 30 minutes. Washes were performed by using PBS plus 0.05% Tween 20.

As shown in FIG. 1A, total IgE levels were significantly elevated in OVA-sensitized and challenged mice treated with the isotype control antibody (Iso. Cont.) (mean IgE levels=3708 ng/ml). The allergen-induced increase in total IgE levels observed in the isotype control mice was significantly reduced in mice treated with either dexamethasone (DEX) (mean IgE levels=1058 ng/ml) or 10F4 (10F4 Ther.) (mean IgE levels=955.1 ng/ml) (FIG. 1A). These data demonstrate that a single dose of a BLyS antagonist (therapeutic group) effectively reduced the allergen-induced increase in total IgE levels within 6 days. Moreover, these data demonstrate that a BLyS antagonist is as effective as classical corticosteroid treatment in reducing the allergen-induced increase in total IgE levels.

Surprisingly, total IgE levels were significantly reduced in the prophylactic group (mice treated with the 10F4 anti-BLyS antibody prior to OVA challenge) (10F4 Pro.) (mean IgE levels=1573 ng/ml) (FIG. 1A). These data demonstrate that treatment with a BLyS antagonist prior to allergen challenge prevented an allergen-induced increase in total IgE levels.

As expected, total IgE levels were very low in mice sensitized and challenged with PBS (mean IgE levels=103.8 ng/ml) and in untreated (naïve) mice (mean IgE levels=26.17 ng/ml) (FIG. 1A).

### OVA-Specific IgE ELISA

OVA-specific serum IgE was detected using MD Bioproducts OVA-IgE ELISA kit (Catalog Number M036005) according to the manufacturer's protocol.

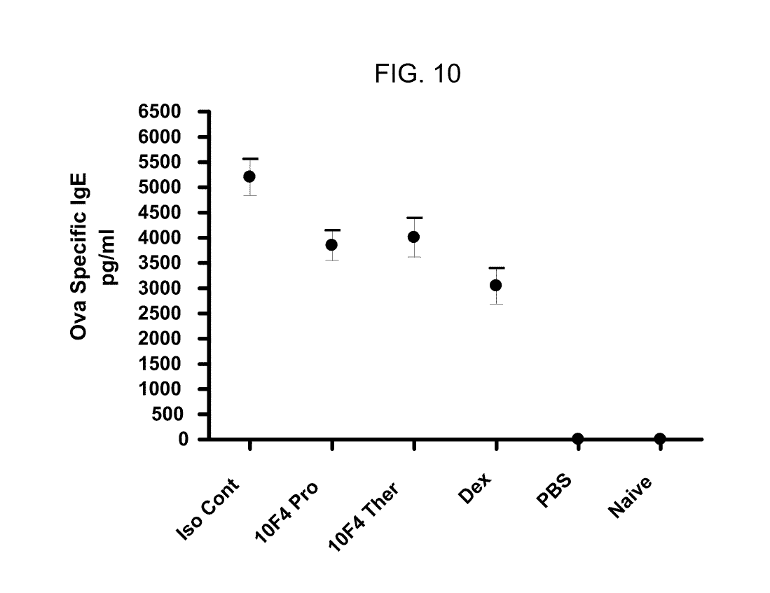

As shown in FIG. 10, OVA-specific IgE levels were significantly elevated in OVA-sensitized and challenged mice treated with the isotype control antibody (Iso. Cont.). The allergen-induced increase in OVA-specific IgE levels observed in the isotype control mice was significantly reduced in mice treated with either dexamethasone (DEX) or 10F4 (10F4 Ther.) (FIG. 10). These data demonstrate that a single dose of a BLyS antagonist (therapeutic group) effectively reduced the allergen-induced increase in OVA-specific (i.e., allergen-specific) IgE levels within 6 days. Moreover, these data demonstrate that a BLyS antagonist is as effective as classical corticosteroid treatment in reducing the allergen-induced increase in allergen-specific IgE levels.

Surprisingly, OVA-specific IgE levels were significantly reduced in the prophylactic group (mice treated with the 10F4 anti-BLyS antibody prior to OVA challenge) (10F4 Pro.) (FIG. 10). These data demonstrate that treatment with a BLyS antagonist prior to allergen challenge prevented an allergen-induced increase in allergen-specific IgE levels.

### Flow Cytometry

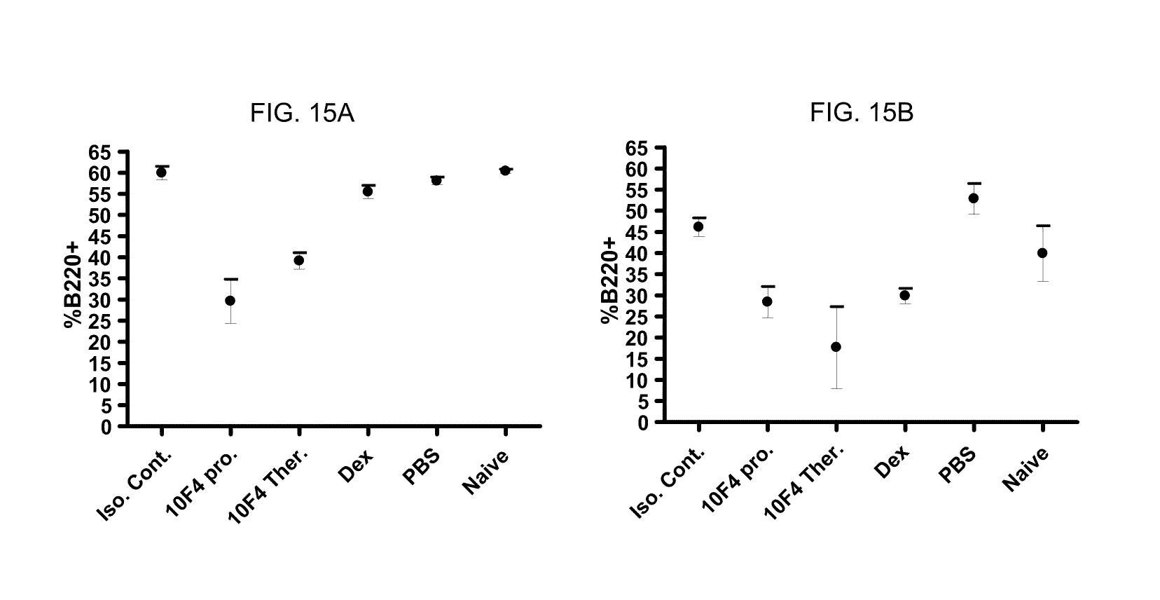

To determine 10F4 modulation of B cells, blood and splenocytes were stained with anti-CD45R/B220 conjugated to Pacific Blue or PE (BD Biosciences). Cells were gated on splenocytes and CD45R/B220+ cells were a percentage of the lymphocyte gate. Data were collected on either a FACs Calibur or a Facs Canto flow cytometer and analyzed using FlowJo (Tree Star). As shown in FIGS. 1B and 15A, the percent of B220+ B cells in the spleen was significantly reduced in the prophylactic (10F4 Pro.) and therapeutic (10F4 Ther.) groups, confirming that treatment with anti-BLyS mAb 10F4 led to in vivo reduction of B220+ B cells in the spleen as expected. Similarly, a decrease in the percent of B220+ B cells was detected in the blood of mice from the prophylactic (10F4 Pro.) and therapeutic (10F4 Ther.) groups (FIGS. 1C and 15B). Similar amounts of B220+ B cells were detected in the spleen of the isotype control, dexamethasone, PBS and naïve groups of mice (FIGS. 1B and 15A), although a decrease in the percent of B220+ B cells was detected in the blood of mice treated with dexamethasone as compared to the isotype control, PBS, and naïve groups of mice (FIGS. 1C and 15B).

### Lung Histology

Lungs were analyzed by histology 24 hours after the last nebulization with either OVA or PBS to determine the effect of 10F4 treatment. Lungs were inflated with 10% formaline, fixed in 10% formaline, dehydrated, mounted in paraffin, sectioned, and stained with hematoxylin/eosin. Airway mucus was identified by the alcian blue/periodic acid-Schiff (PAS) reaction by using a standard protocol.

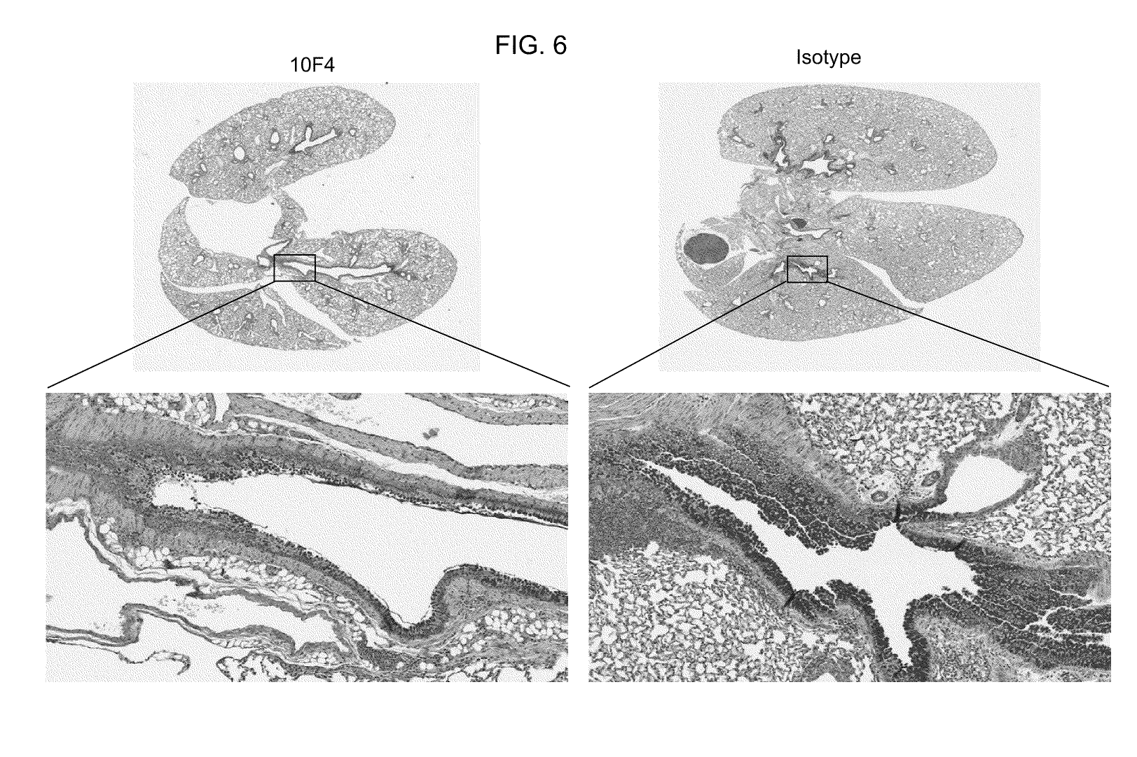

As shown in FIG. 6, mucus accumulation (red staining) and cellular infiltration (blue staining) was significantly reduced in mice treated with 10F4 on day 34 (10F4 therapeutic group) as compared to mice treated with the isotype control antibody on day 34. These data demonstrate that treatment with a B Lymphocyte Stimulator antagonist inhibited allergen-induced increase in mucus accumulation, mucus-containing cells, or general cellular infiltration of immune cells in the lung.

### Lung Infiltrate Analysis

In order to quantify the amount of cellular infiltration in the lung samples, a specific algorithm, Genie, from Aperio, was utilized. Briefly, lung samples were stained with Periodic Acid Shiff staining and hematoxylin using a standard protocol. The stained lung tissues were scanned at 20× magnification using Aperio CS system in order to acquire microscopic digital images. The images were then analyzed using Genie to identify and measure the areas of interest as classified by the investigator. Using positive control slides, Genie was programmed to detect the area of infiltrates based on cell density and morphology. After Genie was tuned for infiltrate measure, each lung tissue was outlined to obtain the total area of lung, and then the area of infiltrates was measured. The percentage of infiltrates in the total area of lung was used for the comparison among groups. Each group had an n=4 except for the PBS and naïve control groups, which had an n=2. The total area of each lung analyzed was 94+2.1 mm² (mean±SEM). An unpaired t-test was used for statistical analysis.

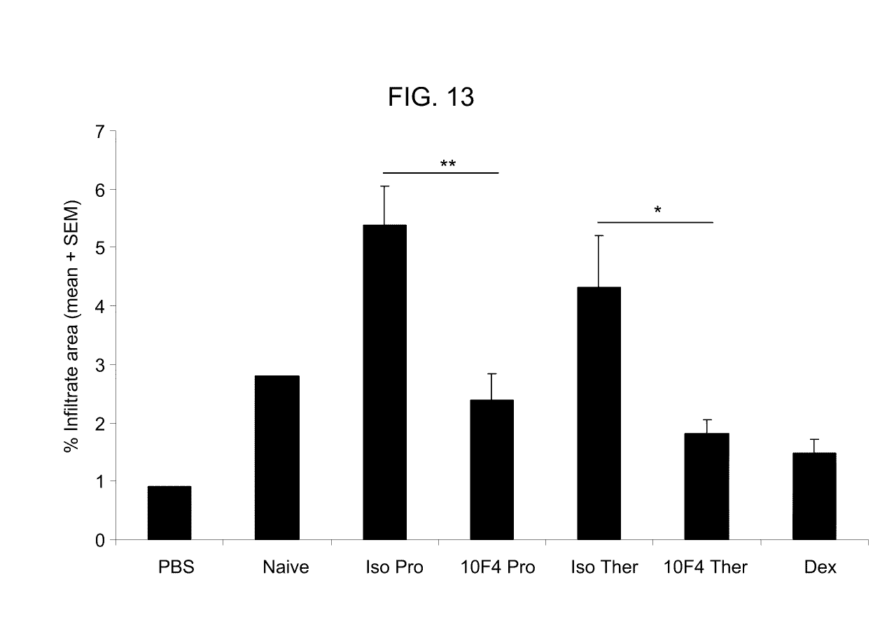

As shown in FIG. 13, cellular infiltration in the lungs was significantly elevated in OVA-sensitized and challenged mice treated with the isotype control antibody either on days 20 and 27 (prophylactic group) (Iso. Pro.) or on day 34 (therapeutic group) (Iso. Ther.) as compared to mice sensitized and challenged with PBS and untreated (naïve) mice (FIG. 13). The * represents a p value <0.05 and the ** represents a p<0.01 (FIG. 13).

The allergen-induced increase in cellular infiltration observed in mice treated with the isotype control antibody on day 34 (Iso. Ther.) was significantly reduced in mice treated with either 10F4 on day 34 (10F4 Ther.) (therapeutic group) or dexamethasone (Dex) (FIG. 13). These data demonstrate that a single dose of a BLyS antagonist (therapeutic group) effectively reduced the allergen-induced increase in cellular infiltration in the lungs within 6 days. Moreover, these data demonstrate that a BLyS antagonist is as effective as classical corticosteroid treatment in reducing the allergen-induced increase in cellular infiltration of immune cells in the lungs.

Cellular infiltration was significantly reduced in the prophylactic group (mice treated with the 10F4 anti-BLyS antibody prior to OVA challenge on days 20 and 27) (10F4 Prophylactic) as compared to the isotype control prophylactic group (mice treated with the isotype control antibody prior to OVA challenge on days 20 and 27) (FIG. 13). These data demonstrate that treatment with a BLyS antagonist prior to allergen challenge prevented allergen-induced increase in cellular infiltration of immune cells in the lung.

### Neutrophil, Eosinophil, and Mast Cell Counts

Upon termination, bronchoalveolar lavage (BAL) fluid was collected from the lungs and then the lungs were inflated with 10% formalin. The lungs were then placed in formalin and fixed, and stained with hematoxylin/eosin. Hematoxylin/eosin stained lung sections from (1) mice sensitized and challenged with PBS, (2) animals treated with 10F4 on day 34 (10F4 Ther) (therapeutic group), and (3) animals treated with isotype control antibody on day 34 (Isotype Ther) were sent out to Charles River Laboratories, Pathology Associates (Frederick, Md.) for neutrophil, eosinophil, and mast cell counts. All counts were performed by Dr. Julia Baker, BVMS, DipRCPath, MRCVS. The counts were performed as using the following procedure. Ten fields of lung parenchyma per section were examined as 40× magnification and the number of neutrophils, eosinophils, and mast cells in each field were counted. The mean number of cells per field was then calculated. The diameter (D) was measured as 200 μm and the area (A) calculated as 31416 μm², using the formula A-π(D/2)². The number of cells/mm² was derived using the formula (MEAN/31416)×1000000.

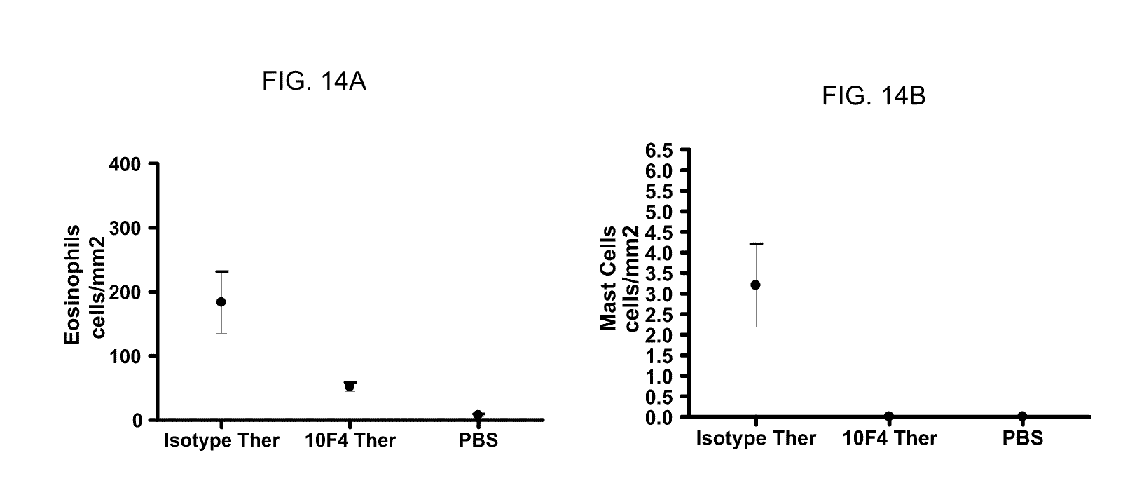

As shown in FIGS. 14A-B and 19A-B, mice therapeutically treated with 10F4 on day 34 had significantly less neutrophils, eosinophils, and mast cells in the lungs as compared to mice treated with the isotype control. These data demonstrate that a single dose of a BLyS antagonist (therapeutic group) effectively reduced the allergen-induced increase in eosinophil and mast cell migration into the lungs within 6 days.

The results of the experiments reflected in this example demonstrate that in vivo BLyS neutralization can ameliorate and/or treat allergen-induced allergic or inflammatory conditions of the lung by reducing total IgE levels, mucus accumulation and infiltration of cells in the lung. In particular, administration of a neutralizing anti-BLyS monoclonal antibody (mAb) in a murine asthma model prevented the onset of lung inflammation when administered before exposure to an allergen known to induce lung inflammation and prevented, ameliorated or treated lung inflammation when administered after exposure to an allergen known to induce lung inflammation. The results of the experiment reflected in this example demonstrate that use of a BLyS antagonist such as an anti-BLyS mAb is a potential therapeutic for the treatment or prevention of an allergic or inflammatory condition of the lung such as asthma.

### Example 5

This example demonstrates that repeated administration of a neutralizing anti-BLyS mAb in a murine model of asthma decreased, treated, prevented and/or ameliorated lung inflammation by reducing total IgE levels, mucus accumulation and infiltration of cells in the lung.

### Ovalbumin Sensitization and Challenge

Female BALB/c mice (8-10 weeks of age) were sensitized with intraperitoneal (i.p.) injections of 20 μg (0.1 ml of 200 μg/ml in PBS) ovalbumin (OVA) (Sigma-Aldrich) every other day from day 1 through 19. Mice were challenged by nebulization with 10 mg/ml OVA in PBS for 40 minutes on days 33, 35, 37, 39, 47, 49, 51, 53, 61, 63, 65, and 67. The mice were terminated either on day 54 or day 68, after the last ovalbumin challenge.

### 10F4 Treatment in OVA-Challenged Mice

To assess the ability of a B Lymphocyte Stimulator antagonist to decrease, treat, prevent and/or ameliorate lung inflammation when administered concomitantly with allergen challenge, mice were treated with either 100 μg of monoclonal IgG1 hamster anti-mouse BLyS (clone 10F4) or an isotype control antibody by i.p. injection on days 34, 41, 48, and 55 (i.e., during OVA challenge). Thus, mice terminated on day 54 received a total of three doses of 10F4 and mice terminated on day 68 received a total of four doses of 10F4.

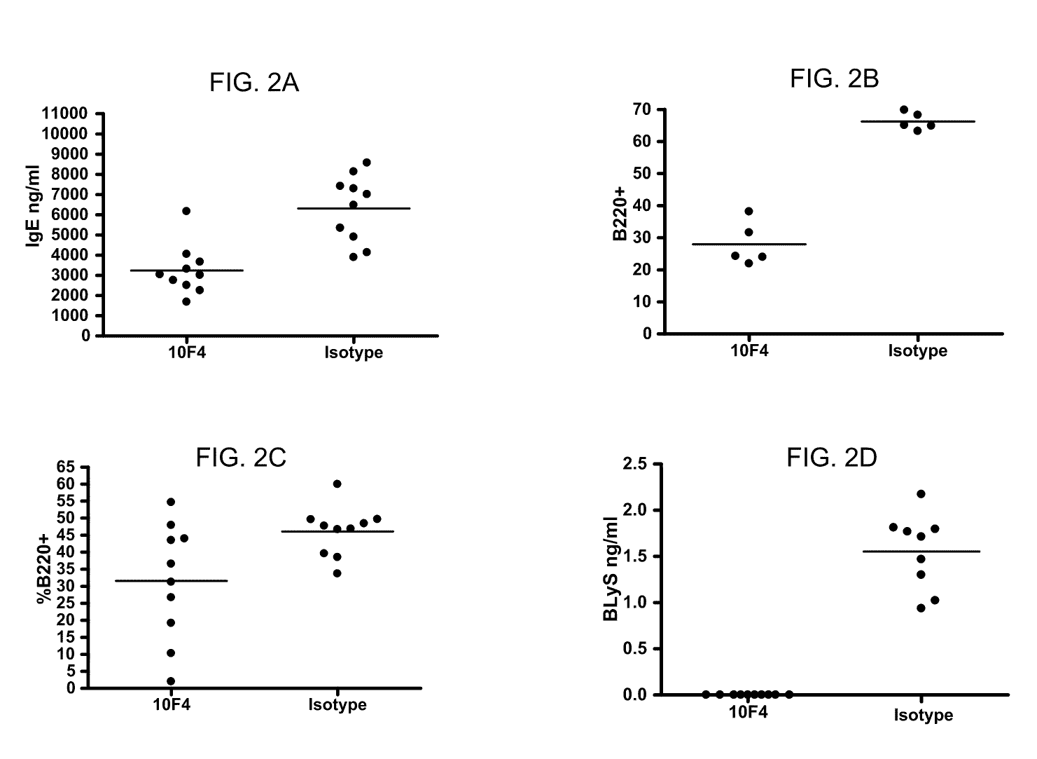

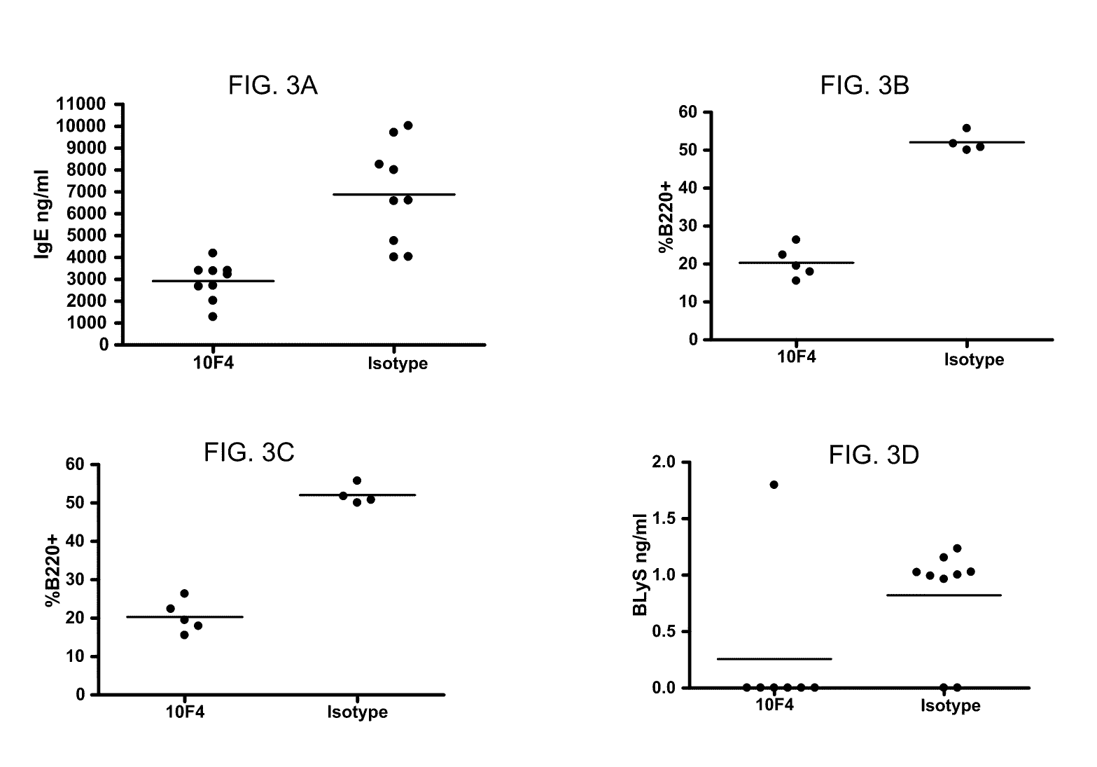

As expected, there was a significant reduction in serum BLyS levels in mice treated either three (FIG. 2D) or four (FIG. 3D) times with 10F4 as compared to mice treated with isotype control antibody. These data confirm that treatment with anti-BLyS mAb 10F4 led to in vivo BLyS neutralization.

### Total IgE ELISA

Serum IgE was detected as described in Example 4 using BD Biosciences Mouse OptEIA Total IgE ELISA Kit. As expected, total IgE levels were significantly elevated in mice treated three times with the isotype control antibody (Isotype) (mean IgE levels=6313 ng/ml) (FIG. 2A). In contrast, total IgE levels were significantly reduced in mice treated three times with 10F4 (10F4) (mean IgE levels=3243 ng/ml) (FIG. 2A). Similarly, total IgE levels were significantly reduced in mice treated four times with 10F4 (10F4) (mean IgE levels=2917 ng/ml) as compared to mice treated four times with the isotype control antibody (Isotype) (mean IgE levels=6882 ng/ml) (FIG. 3A). These data demonstrate that repeated administration of a BLyS antagonist during several rounds of allergen challenge effectively reduced the allergen-induced increase in total IgE levels.

### Flow Cytometry

CD45R/B220+ cells were detected by flow cytometry as described in Example 4. The percent of B220+ B cells in the spleen (FIG. 2B) and blood (FIG. 2C) was significantly reduced in mice treated three times with 10F4 (10F4) as compared to mice treated three times with the isotype control antibody (Isotype). Similarly, the percent of B220+ B cells in the spleen (FIG. 3B) and blood (FIG. 3C) was significantly reduced in mice treated four times with 10F4 (10F4) as compared to mice treated four times with the isotype control antibody (Isotype). These results confirm that treatment with anti-BLyS mAb 10F4 led to a reduction of B-cells in vivo.

### Lung Histology

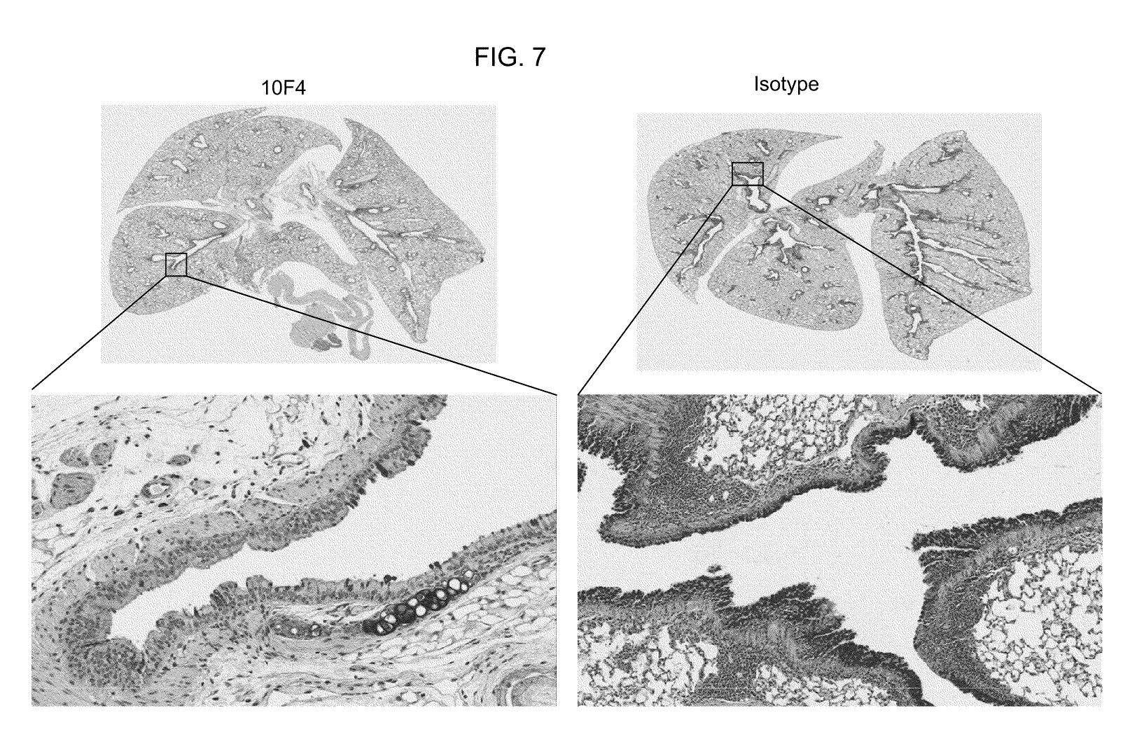

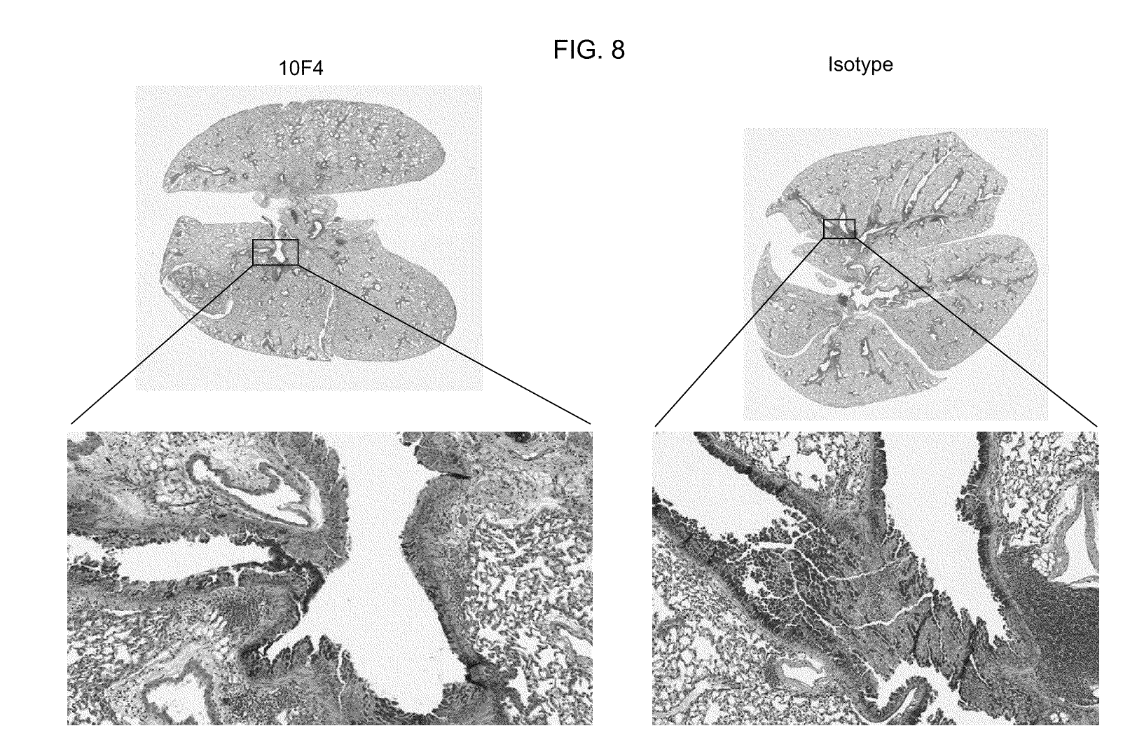

Lungs were analyzed by histology as described in Example 4. As shown in FIG. 7, mucus accumulation (red staining) and cellular infiltration (blue staining) was significantly reduced in mice treated three times with 10F4 (10F4) as compared to mice treated with the isotype control antibody (Isotype). Similar results were observed in mice treated four times with 10F4 (FIG. 8). These data demonstrate that repeated treatment with a B Lymphocyte Stimulator antagonist during several rounds of allergen challenge inhibited allergen-induced increase in mucus accumulation, mucus-containing cells, or general cellular infiltration of immune cells in the lung.

The results of the experiments reflected in this example demonstrate that repeated therapeutic administration of a neutralizing anti-BLyS monoclonal antibody (mAb) in a murine model of asthma decreased, prevented, ameliorated and/or treated allergen-induced allergic or inflammatory conditions of the lung by reducing total IgE levels, mucus accumulation and infiltration of cells in the lung. The results of the experiment reflected in this example demonstrate that use of a BLyS antagonist such as an anti-BLyS mAb is a potential therapeutic for the treatment or prevention of an allergic or inflammatory condition of the lung such as asthma.

### Example 6

This example demonstrates that therapeutic administration of a neutralizing anti-BLyS mAb in a murine model of asthma reduced total IgE levels, mucus accumulation and infiltration of cells in the lung 72 days after the last administration of the neutralizing anti-BLyS mAb.

### Ovalbumin Sensitization and Challenge

Female BALB/c mice (8-10 weeks of age) were sensitized with intraperitoneal (i.p.) injections of 20 μg (0.1 ml of 200 μg/ml in PBS) ovalbumin (OVA) (Sigma-Aldrich) every other day from day 1 through 19. Mice were challenged by nebulization with 10 mg/ml OVA in PBS for 40 minutes on days 33, 35, 37, 39, 47, 49, 51, 53, 61, 63, 65, 67, 120, 122, 124, and 126. The mice were terminated on day 127, after the last ovalbumin challenge. Control mice were either sensitized and challenged with PBS or left untreated (naïve) and terminated on day 127.

### 10F4 Treatment in OVA-Challenged Mice

To assess the ability of a B Lymphocyte Stimulator antagonist to decrease, treat, prevent and/or ameliorate chronic allergen-induced lung inflammation, mice were treated with either 100 μg of monoclonal IgG1 hamster anti-mouse BLyS (clone 10F4) or an isotype control antibody by i.p. injection on days 34, 41, 48, and 55 (i.e., during OVA challenge). Thus, the mice received a total of four doses of 10F4 wherein the last administration of 10F4 was given 72 days prior to termination.

### Total IgE ELISA

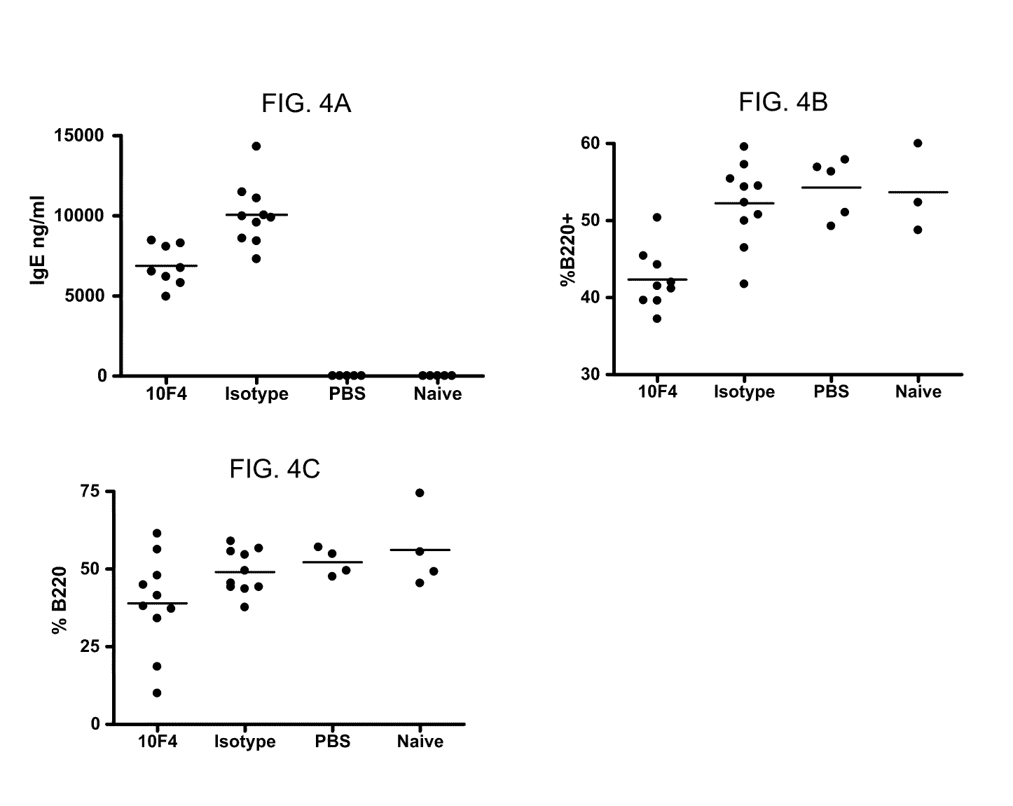

Serum IgE was detected as described in Example 4 using BD Biosciences Mouse OptEIA Total IgE ELISA Kit. Total IgE levels were significantly elevated in mice treated with the isotype control antibody on day 127 (Isotype) (mean IgE levels=10062 ng/ml) (FIG. 4A). Surprisingly, the total IgE levels were significantly reduced in mice treated with 10F4 (10F4) (mean IgE levels=6878 ng/ml) (FIG. 4A) on day 127 despite the discontinuation of the anti-BLyS antibody treatment on day 55. These data demonstrate that repeated treatment with a BLyS antagonist effectively protected against the allergen-induced increase in IgE levels up to 72 days after the last administration of the BLyS antagonist. The reduction in allergen-induced increase in total IgE levels were observed even though mice were challenged 8 more times with OVA following the final administration of an anti-BLyS antibody.

As shown in FIG. 4A, total IgE was undetectable in mice sensitized and challenged with PBS (mean IgE levels=Ong/ml) and in untreated (naïve) mice (mean IgE levels=Ong/ml), as expected.

### Flow Cytometry

CD45R/B220+ cells were detected by flow cytometry as described in Example 4. The percent of B220+ B cells in the spleen (FIG. 4B) and blood (FIG. 4C) was significantly reduced in mice treated with 10F4 (10F4) as compared to mice treated with the isotype control antibody (Isotype), mice sensitized and challenged with PBS (PBS), and untreated mice (naïve). These results confirm that treatment with anti-BLyS mAb 10F4 led to a reduction of B-cells in vivo.

### Lung Histology

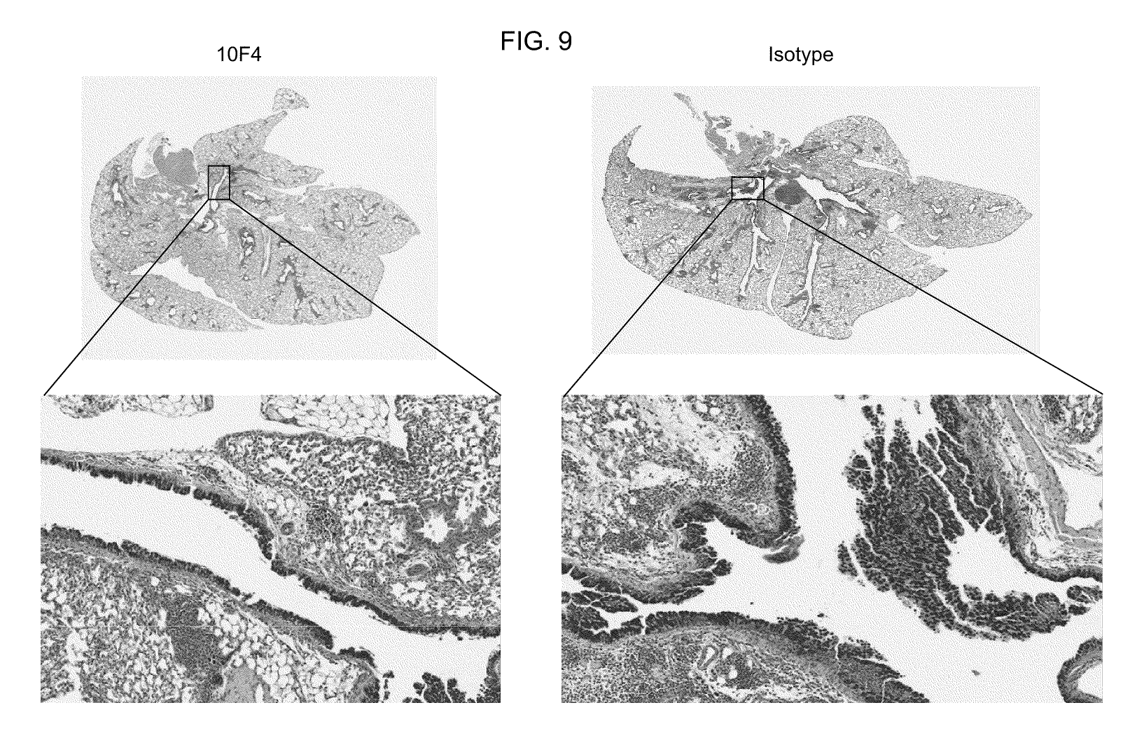

Lungs were analyzed by histology as described in Example 4. As shown in FIG. 9, mucus accumulation (red staining) and cellular infiltration (blue staining) was significantly reduced in mice treated with 10F4 (10F4) as compared to mice treated with the isotype control antibody (Isotype). These data demonstrate that repeated treatment with a B Lymphocyte Stimulator antagonist inhibited allergen-induced increase in mucus accumulation, mucus-containing cells in the lung, or general cellular infiltration of immune cells in the lung up to 72 days after the last administration of the BLyS antagonist.

The results of the experiments reflected in this example demonstrate that repeated therapeutic administration of a neutralizing anti-BLyS monoclonal antibody (mAb) in a murine model of asthma reduced total IgE levels, mucus accumulation and infiltration of cells in the lung 72 days after the last administration of the neutralizing anti-BLyS mAb. The results of the experiment reflected in this example demonstrate that use of a BLyS antagonist such as an anti-BLyS mAb is a potential therapeutic for the treatment, prevention and/or amelioration of an allergic or inflammatory condition of the lung such as asthma. Furthermore, results of the experiments reflected in this example demonstrate that repeated therapeutic administration of a neutralizing anti-BLyS monoclonal antibody (mAb) significantly delays onset of allergen-induced lung inflammation and is therefore a potential therapeutic for the treatment or prevention of chronic allergic or inflammatory conditions of the lung.

### Example 7

This example demonstrates that in vivo B Lymphocyte Stimulator (BLyS) protein neutralization has a long-lasting effect in reducing total IgE levels in a murine model of asthma up to 104 days after administration.

### Ovalbumin Sensitization and Challenge

Female BALB/c mice (8-10 weeks of age) were sensitized with intraperitoneal (i.p.) injections of 20 μg (0.1 ml of 200 m/ml in PBS) ovalbumin (OVA) (Sigma-Aldrich) every other day from day 1 through 19. Mice were challenged by nebulization with 10 mg/ml OVA in PBS for 40 minutes on days 33, 35, 37, 39, 47, 49, 51, 53, 61, 63, 65, 67, 120, 122, 124, 126, 152, 154, 156, and 158. The mice were terminated on day 159, after the last ovalbumin challenge.

### 10F4 Treatment in OVA-Challenged Mice

To assess the long-term ability of a B Lymphocyte Stimulator antagonist to prevent, treat or ameliorate chronic lung inflammation, mice were treated with 100 μg of monoclonal IgG1 hamster anti-mouse BLyS (clone 10F4) by i.p. injection on days 34 and 41 (2× 10F4) or on days 34, 41, 48, and 55 (4× 10F4). Control mice were left untreated (No Rx).

### Total IgE ELISA

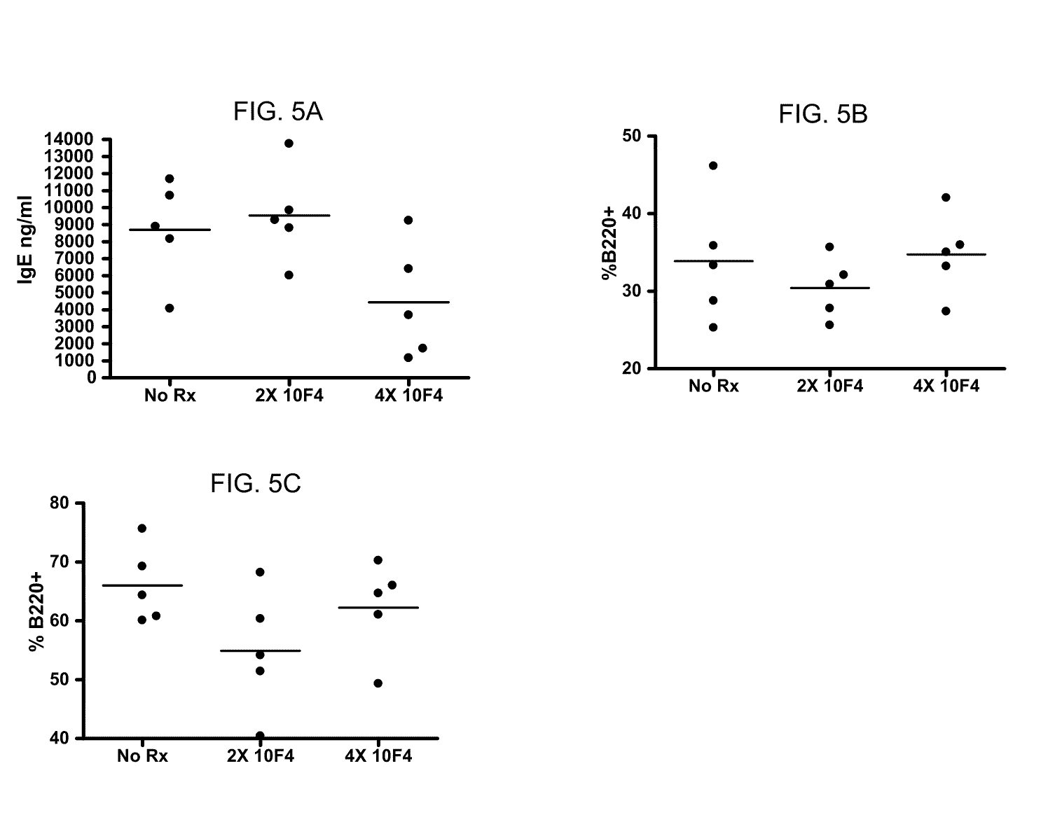

Serum IgE was detected as described in Example 4 using BD Biosciences Mouse OptEIA Total IgE ELISA Kit. As shown in FIG. 5A, total IgE levels were significantly reduced in mice treated four times with 10F4 (4× 10F4) (mean IgE levels=4439 ng/ml) as compared to the untreated mice (No Rx) (mean IgE levels=8697 ng/ml) and mice treated two times with 10F4 (2× 10F4) (mean IgE levels=9534 ng/ml). Surprisingly, these data demonstrate that treatment with a BLyS antagonist effectively protected against the allergen-induced increase in IgE levels for up to 104 days after the last administration. The reduction in allergen-induced increase in total IgE levels in mice treated four times with 10F4 (4×10F4) were observed even though mice were challenged 12 more times with OVA following the final administration of an anti-BLyS antibody.

### Flow Cytometry

CD45R/B220+ cells were detected by flow cytometry as described in Example 4. The percent of B220+ B cells in the spleen is depicted in FIG. 5B for each treatment group and the percent of B220+ B cells in the blood is depicted in FIG. 5C for each treatment group. As previously reported, the half-life of 10F4 in vivo is z2 weeks (see Schoz et al. Proc Natl Acad Sci USA. 2008 Oct. 7; 105(40): 15517-15522). Accordingly, after over 100 days following the last treatment with 10F4, little, if any, 10F4 antibody should be active in the mice treated two times or four times with 10F4. As shown in FIGS. 5B and 5C, by day 159, B220+ B cells levels in mice treated two times or four times with 10F4 had returned to normal, consistent with the assumption that little, if any, 10F4 antibody was active in these mice. Additionally, B cells returning to pre-10F4 treated levels also provides evidence that there was a complete reconstitution of B cells. Surprisingly, as shown in FIG. 5A, mice treated four times with 10F4 (4× 10F4) maintained a significant reduction in total IgE levels up to day 159 as compared to the untreated mice.

The results of the experiments reflected in this example demonstrate that in vivo B Lymphocyte Stimulator (BLyS) protein neutralization has a long-lasting effect in the prevention, treatment or amelioration of chronic lung inflammation. In particular, repeated administration of a neutralizing anti-BLyS mAb in a murine model of asthma significantly reduced total IgE levels in mice treated four times with 10F4 up to 104 days after administration. The results of the experiment reflected in this example demonstrate that use of a BLyS antagonist such as an anti-BLyS mAb is a potential therapeutic for the treatment, prevention, and/or amelioration of an allergic or inflammatory condition of the lung such as asthma. Furthermore, results of the experiments reflected in this example demonstrate that repeated therapeutic administration of a neutralizing anti-BLyS monoclonal antibody (mAb) significantly delays onset of allergen-induced lung inflammation and is therefore a potential therapeutic for the treatment or prevention of chronic allergic or inflammatory conditions of the lung.

### Example 8

This example demonstrates that in vivo B Lymphocyte Stimulator (BLyS) protein neutralization can prevent, ameliorate and/or treat allergen-induced allergic or inflammatory conditions of the lung as effectively as dexamethasone or an anti-IL-13 antibody.

IL-13 is a cytokine secreted by many cell types, but especially T helper type 2 (Th2) cells, that is an important mediator of allergic inflammation and disease. IL-13 induces many features of allergic lung disease, including airway hyper responsiveness, goblet cell metaplasia and mucus hypersecretion, which all contribute to airway obstruction. IL-4 contributes to these physiologic changes, but is less important than IL-13. IL-13 also induces secretion of chemokines that are required for recruitment of allergic effector cells to the lung.

### Ovalbumin Sensitization and Challenge

Female BALB/c mice (8-10 weeks of age) were sensitized with intraperitoneal (i.p.) injections of 20 μg (0.1 ml of 200 m/ml in PBS) ovalbumin (OVA) (Sigma-Aldrich) every other day from day 1 through 19. Starting at day 33, the mice were challenged by nebulization with 10 mg/ml ovalbumin in PBS for 40 minutes every other day for four days, (i.e., the mice were challenged on days 33, 35, 37, and 39). The mice were terminated on day 40, after the last ovalbumin challenge. Control mice were either sensitized and challenged with PBS or left untreated (naïve) and terminated on day 40. Upon termination, blood, lung, bronchoalveolar lavage (BAL) fluid, and spleen were removed from the animals. In addition, the blood was centrifuged and the serum was removed.

### 10F4, Anti-IL-13, Anti-IgE, and Dexamethasone Treatment in OVA-Sensitized Mice

To assess the ability of a B Lymphocyte Stimulator antagonist to prevent the onset of lung inflammation, one group of OVA-sensitized mice was treated with 100 μg of monoclonal IgG1 hamster anti-mouse BLyS (clone 10F4) by i.p. injection on days 20 and 27, prior to challenge with OVA (10F4 prophylactic group). In order to compare the efficacy of 10F4 to another antibody treatment, one group of OVA-sensitized mice was treated with 100 μg of an anti-IL-13 antibody (R&D Systems; Cat. #MAB413) by i.p. injection on days 20 and 27, prior to challenge with OVA (IL-13 prophylactic group). As a control, one group of OVA-sensitized mice was treated with 100 μg of an isotype control antibody (purified Armenian hamster IgG1 (clone G235-2356; BD Biosciences)) by i.p. injection on days 20 and 27, prior to challenge with OVA (isotype control prophylactic group).

To assess the ability of a B Lymphocyte Stimulator antagonist to prevent, ameliorate or treat lung inflammation, one group of OVA-sensitized mice was treated with 100 μg of monoclonal IgG1 hamster anti-mouse BLyS (clone 10F4) by i.p. injection on day 34 (i.e., during OVA challenge) (10F4 therapeutic group). In order to compare the efficacy of 10F4 to another antibody treatment, one group of OVA-sensitized mice was treated with 100 μg of an anti-IL-13 antibody (R&D Systems; Cat. #MAB413) by i.p. injection on day 34 (i.e., during OVA challenge) (IL-13 therapeutic group). As controls, one group of OVA-sensitized mice was treated with 100 μg of an anti-IgE antibody (BD Pharmingen; Cat. #553416) by i.p. injection on day 34 (i.e., during OVA challenge) (IgE therapeutic group) and one group of OVA-sensitized mice was treated with 100 μg of an isotype control antibody (purified Armenian hamster IgG1 (clone G235-2356; BD Biosciences)) by i.p. injection on day 34 (isotype control therapeutic group).

To compare the efficacy of 10F4 to classical treatment with glucocorticoids, one group of OVA-sensitized mice was given water soluble dexamethasone (DEX) (Sigma-Aldrich) (50 μg in 100 μl of PBS) by i.p. injection every other day of OVA challenge (i.e., dexamethasone was administered on days 34, 36, and 38).

The PBS and naïve groups of mice were left untreated.

All mice were terminated on day 40.

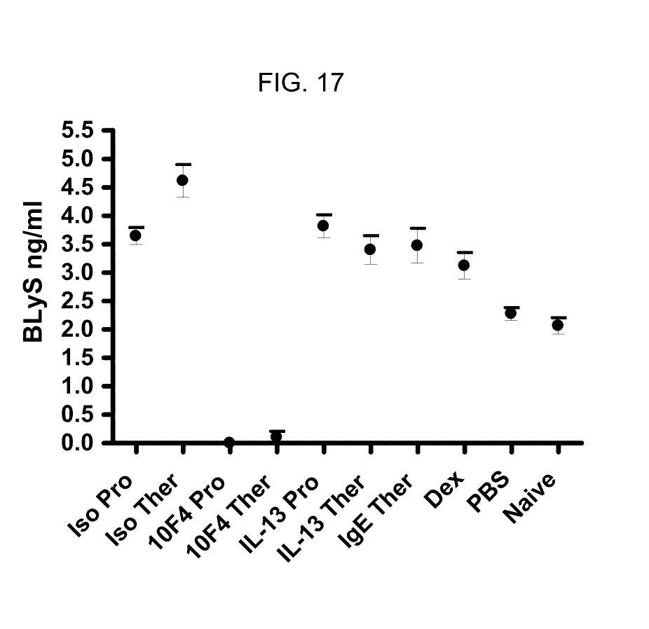

As shown in FIG. 17, there was a significant reduction in serum BLyS levels in the 10F4 prophylactic group (10F4 Pro) or the 10F4 therapeutic group (10F4 Ther), as expected. The remaining treatment regimens exhibited varying effects on circulating B Lymphocyte Stimulator levels, as shown in FIG. 17.

### Total IgE ELISA

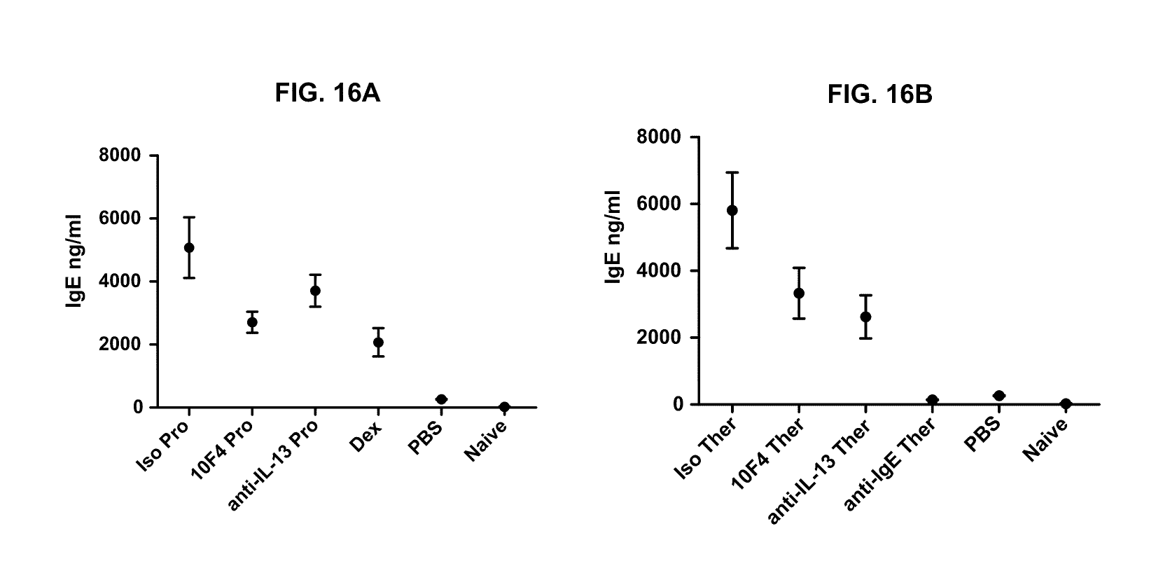

Serum IgE was detected using a total IgE ELISA developed by Human Genome Sciences, Inc. (Rockville, Md.). As expected, total IgE levels were significantly elevated in OVA-sensitized and challenged mice prophylactically (Iso Pro) or therapeutically (Iso Ther) treated with the isotype control antibody as compared to mice sensitized and challenged with PBS and untreated (naïve) mice (FIG. 16A-B).

The allergen-induced increase in total IgE levels observed in the isotype control mice was significantly reduced in mice therapeutically treated on day 34 with 10F4 (10F4 Ther) or anti-IL-13 (IL-13 Ther), and in dexamethasone treated mice (Dex) (FIG. 16A-B). These data demonstrate that a single dose of a BLyS antagonist (therapeutic group) was as effective as dexamethasone or an anti-IL-13 antibody in reducing the allergen-induced increase in total IgE levels within 6 days. Moreover, these data demonstrate that a BLyS antagonist is as effective as classical corticosteroid treatment or an IL-13 antagonist in reducing the allergen-induced increase in total IgE levels.

Total IgE levels were significantly reduced in mice prophylactically treated with 10F4 prior to OVA challenge (10F4 Pro), as compared to a modest reduction in total IgE levels in mice prophylactically treated with anti-IL-13 prior to OVA challenge (IL-13 Pro) (FIG. 16A). These data demonstrate that treatment with a BLyS antagonist prior to allergen challenge prevented an allergen-induced increase in total IgE levels to the same as extent, or better than, treatment with an IL-13 antagonist.

As expected, IgE was not detected in mice therapeutically treated with an anti-IgE antibody (IgE Ther) (FIG. 16B).

### OVA-Specific IgE ELISA

OVA-specific serum IgE was detected using MD Bioproducts OVA-IgE ELISA kit (Catalog Number M036005) according to the manufacturer's protocol.

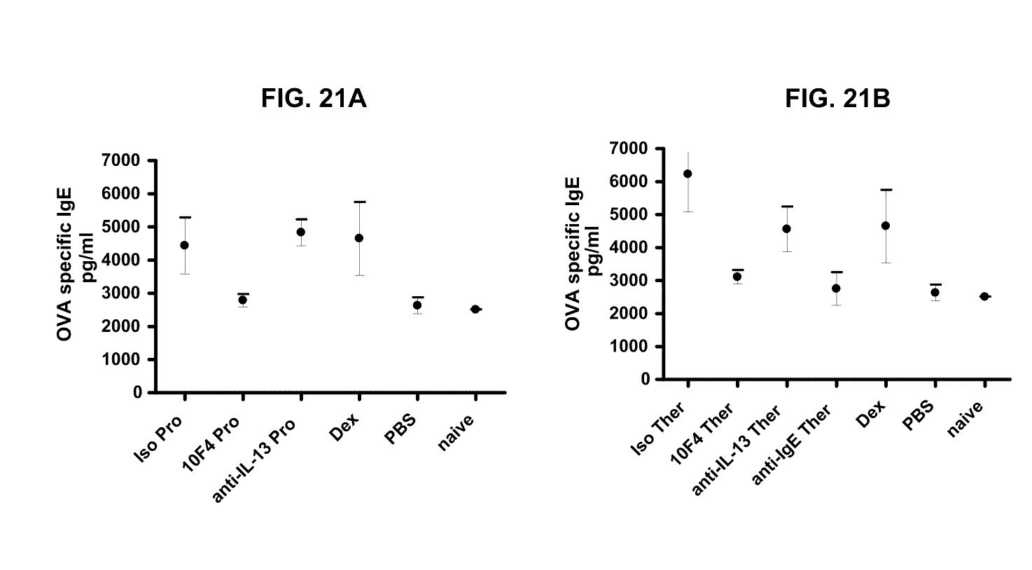

As expected, OVA lung specific IgE levels were significantly elevated in OVA-sensitized and challenged mice prophylactically (Iso Pro) or therapeutically (Iso Ther) treated with the isotype control antibody as compared to mice sensitized and challenged with PBS and untreated (naïve) mice (FIG. 21A-B).

The allergen-induced increase in OVA lung specific IgE levels observed in the isotype control mice was significantly reduced in mice therapeutically treated on day 34 with 10F4 (10F4 Ther) or anti-IgE antibody (anti-IgE Ther) as compared to a modest reduction in OVA lung specific IgE levels in mice therapeutically treated on day 34 with anti-IL-13 antibody (IL-13 Ther) and in dexamethasone treated mice (Dex) (FIG. 21B). These data demonstrate that a single dose of a BLyS antagonist (therapeutic group) was as effective as an anti-IgE antibody in reducing the allergen-induced increase in OVA lung specific IgE levels within 6 days. Moreover, these data demonstrate that a BLyS antagonist is more effective than a classical corticosteroid treatment or an IL-13 antagonist in reducing the allergen-induced increase in OVA lung specific IgE levels.

OVA lung specific IgE levels were significantly reduced in mice prophylactically treated with 10F4 prior to OVA challenge (10F4 Pro) as compared to OVA lung specific IgE levels in mice prophylactically treated with anti-IL-13 prior to OVA challenge (IL-13 Pro) and in dexamethasone treated mice (Dex) (FIG. 21A). These data demonstrate that treatment with a BLyS antagonist prior to allergen challenge prevented an allergen-induced increase in OVA lung specific IgE levels. These data also demonstrate that treatment with an IL-13 antagonist prior to allergen challenge had no effect on the allergen-induced increase in OVA lung specific IgE levels.

### Lung Histology

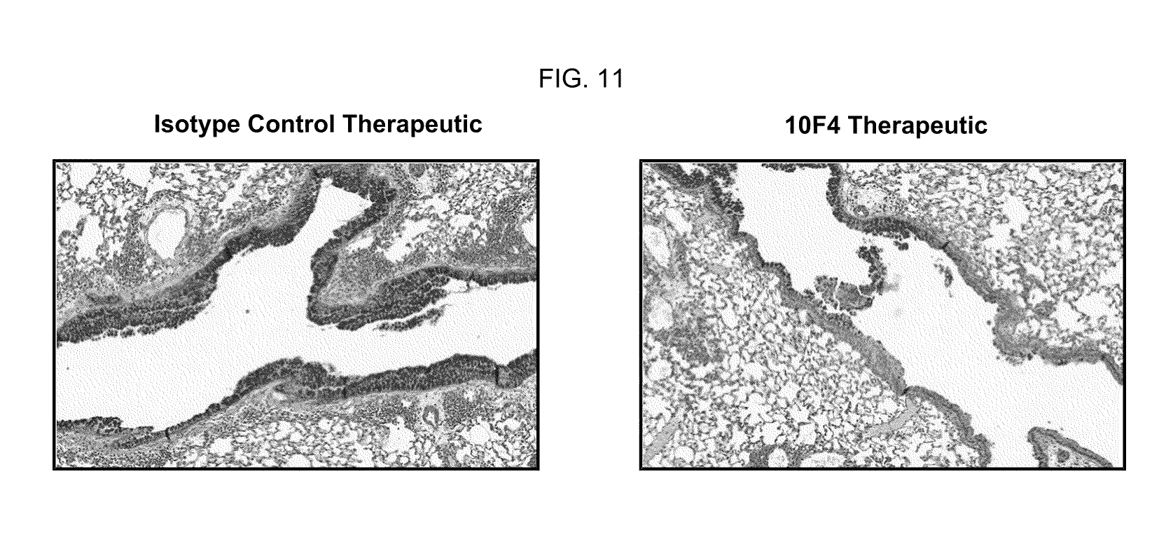

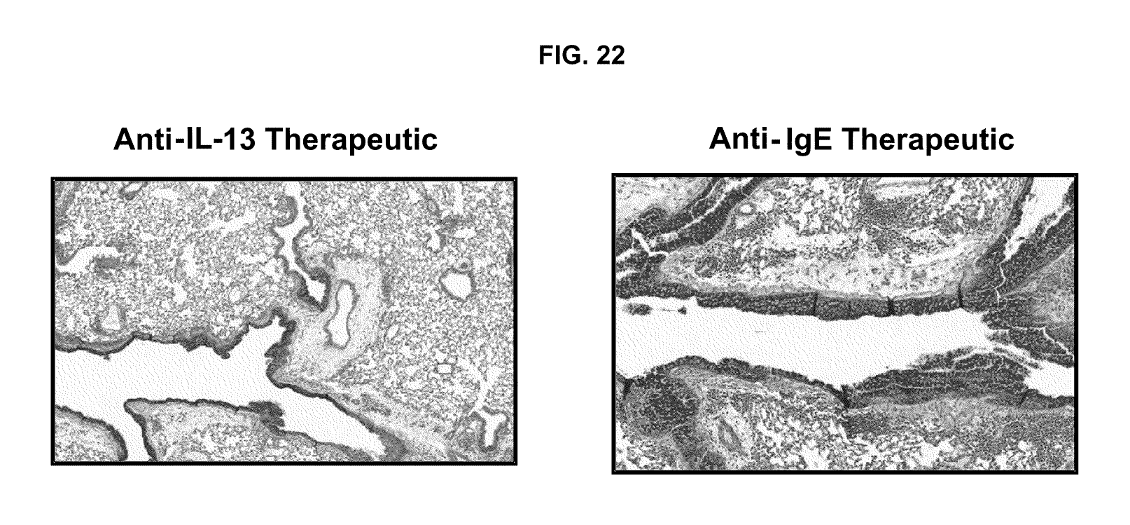

Lungs were analyzed by histology as described in Example 4. As shown in FIGS. 11 and 22, mucus accumulation (red staining) and cellular infiltration (blue staining) was significantly reduced in mice treated on day 34 with 10F4 (10F4 therapeutic) or with anti-IL-13 antibody (anti-IL-13 therapeutic) as compared to mice treated on day 34 with the isotype control antibody (isotype control therapeutic) or anti-IgE antibody (anti-IgE therapeutic). These data demonstrate that treatment with a BLyS antagonist (therapeutic group) was as effective as treatment with an anti-IL-13 antibody in inhibiting allergen-induced increase in mucus accumulation, mucus-containing cells, or general cellular infiltration of immune cells in the lung. Moreover, these data demonstrate that a BLyS antagonist is more effective than an IgE antagonist in inhibiting allergen-induced increase in mucus accumulation, mucus-containing cells, or general cellular infiltration of immune cells in the lung.

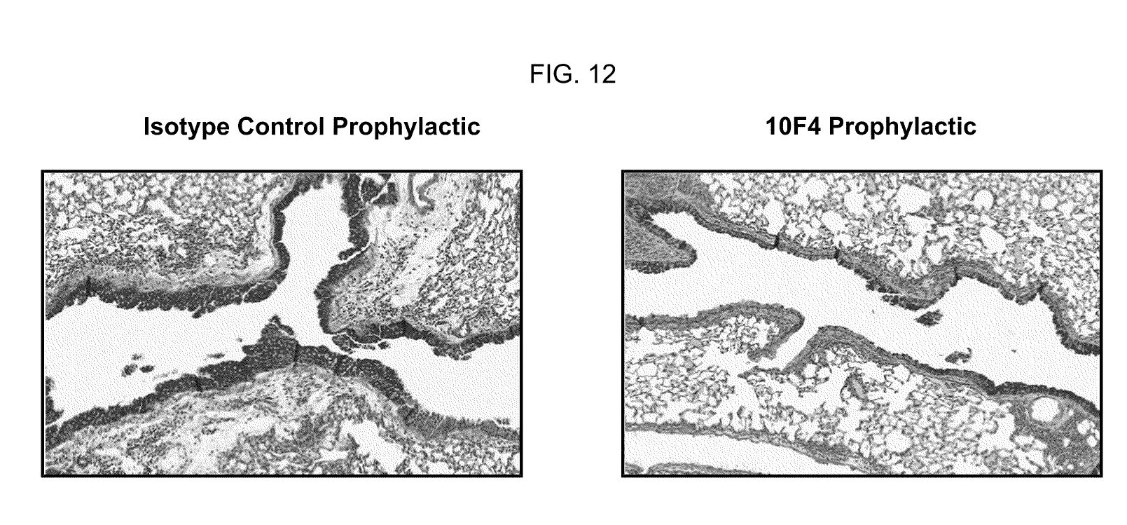

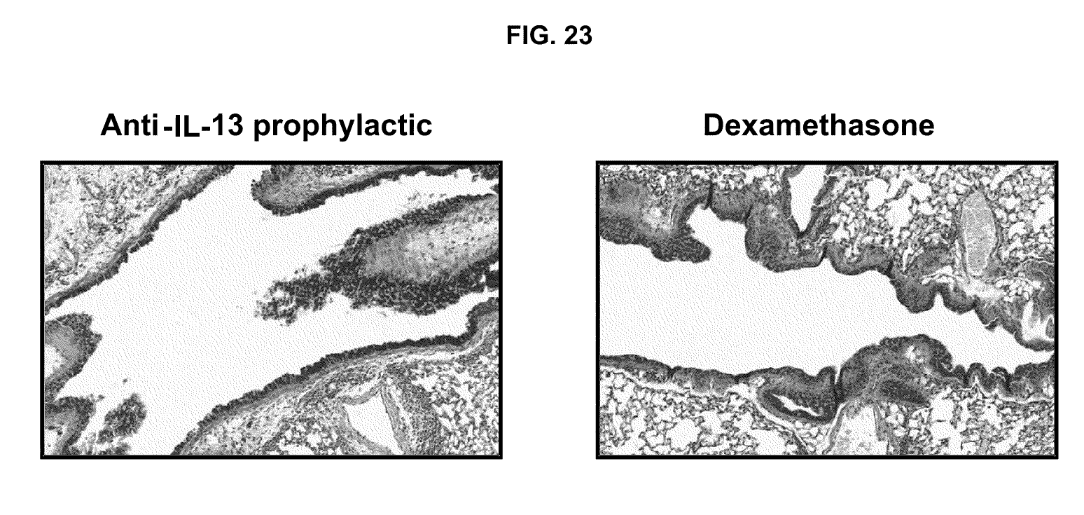

Surprisingly, mucus accumulation (red staining) and cellular infiltration (blue staining) was significantly reduced in mice treated prior to OVA challenge with 10F4 (10F4 prophylactic) (see FIG. 12) as compared to mice treated prior to OVA challenge with an isotype control antibody (isotype control prophylactic) (see FIG. 12) or an anti-IL-13 antibody (anti-IL-13 prophylactic) (see FIG. 23). These data demonstrate that treatment with a BLyS antagonist prior to allergen challenge prevented allergen-induced increase in mucus accumulation, mucus-containing cells, or general cellular infiltration of immune cells in the lung.

In addition, mucus accumulation (red staining) and cellular infiltration (blue staining) was reduced in mice treated with 10F4 on day 34 (10F4 therapeutic) (see FIG. 11) and in mice treated prior to OVA challenge with 10F4 (10F4 prophylactic) (see FIG. 12) as compared to mice treated with dexamethasone (see FIG. 23). These data demonstrate that treatment with a BLyS antagonist was as effective as a classical corticosteroid treatment in preventing or inhibiting allergen-induced increase in mucus accumulation, mucus-containing cells, or general cellular infiltration of immune cells in the lung.

### IL-13 ELISA

IL-13 levels were detected in bronchoalveolar lavage (BAL) fluid using an IL-13 ELISA (eBioscience, cat. #88-7137-22) according to the manufacturer's protocol.

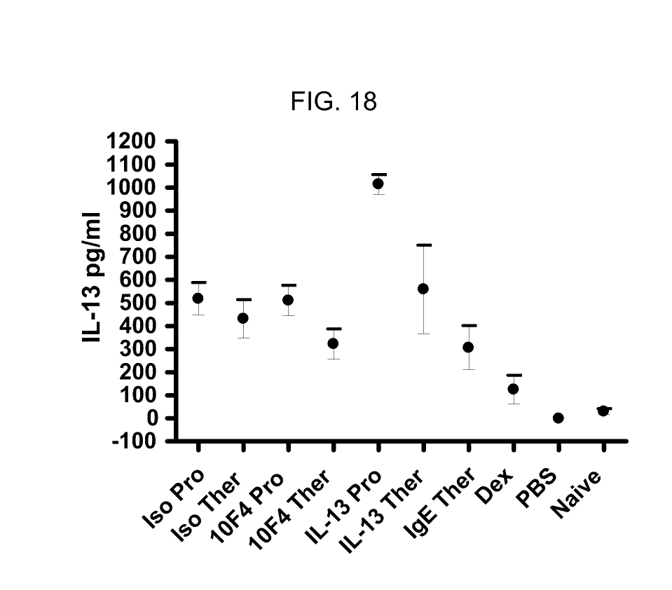

As shown in FIG. 18, there was a slight decrease in IL-13 levels detected in BAL fluid from animals treated therapeutically with 10F4 (10F4-ther) as compared to animals treated prophylactically with 10F4 (10F4-pro), animals treated therapeutically and prophylactically with the isotype control antibody (Iso Pro and Iso Ther, respectively), or animals therapeutically treated with anti-IL-13 antibody (IL-13-ther). In addition, IL-13 levels were decreased in mice treated with dexamethasone (Dex) as compared to all antibody treated groups (FIG. 18).

The results of the experiments reflected in this example demonstrate that in vivo BLyS neutralization can prevent, ameliorate and/or treat allergen-induced allergic or inflammatory conditions of the lung at least as effectively as dexamethasone or an anti-IL-13 antibody. In particular, administration of a neutralizing anti-BLyS monoclonal antibody (mAb) in a murine asthma model reduced total and OVA-specific IgE levels to the same extent, or better than, an anti-IL-13 antibody when administered before or after exposure to an allergen known to induce lung inflammation. In addition, the results of the experiments reflected in this example demonstrate that anti-BLyS treatment resulted in a decrease in specific cytokines in the lung, such as IL-13, that have been routinely implicated in airway inflammation.

### Example 9

This example demonstrates that administration of a B Lymphocyte Stimulator (BLyS) antagonist at the onset of allergen challenge (i.e., 1 or 3 days after the first allergen challenge) can prevent, ameliorate, and/or treat allergen-induced allergic or inflammatory conditions of the lung.

### Ovalbumin Sensitization and Challenge

Female BALB/c mice (8-10 weeks of age) were sensitized with intraperitoneal (i.p.) injections of 20 μg (0.1 ml of 200 μg/ml in PBS) ovalbumin (OVA) (Sigma-Aldrich) every other day from day 1 through 19. Starting at day 33, the mice were challenged by nebulization with 10 mg/ml ovalbumin in PBS for 40 minutes every other day for four days, (i.e., the mice were challenged on days 33, 35, 37, and 39).

### 10F4 Treatment in OVA-Sensitized Mice

To assess whether administration of a B Lymphocyte Stimulator antagonist is effective in preventing, ameliorating or treating lung inflammation during allergen challenge, OVA-sensitized mice were treated with 100 μg of monoclonal IgG1 hamster anti-mouse BLyS (clone 10F4) by i.p. injection either on day 34 (d34 10F4 group), on day 36 (d36 10F4 group), or on day 38 (d38 10F4 group) (i.e., during OVA challenge). As a control, OVA-sensitized mice were treated with 100 μg of an isotype control antibody (purified Armenian hamster IgG1 (clone G235-2356; BD Biosciences)) by i.p. injection either on day 34, on day 36, or on day 38 (isotype group).

All of the mice were terminated on day 40, after the last ovalbumin challenge. Upon termination, blood, lung, bronchoalveolar lavage (BAL) fluid, and spleen were removed from the animals. The blood was centrifuged and the serum was removed.

### Total IgE ELISA

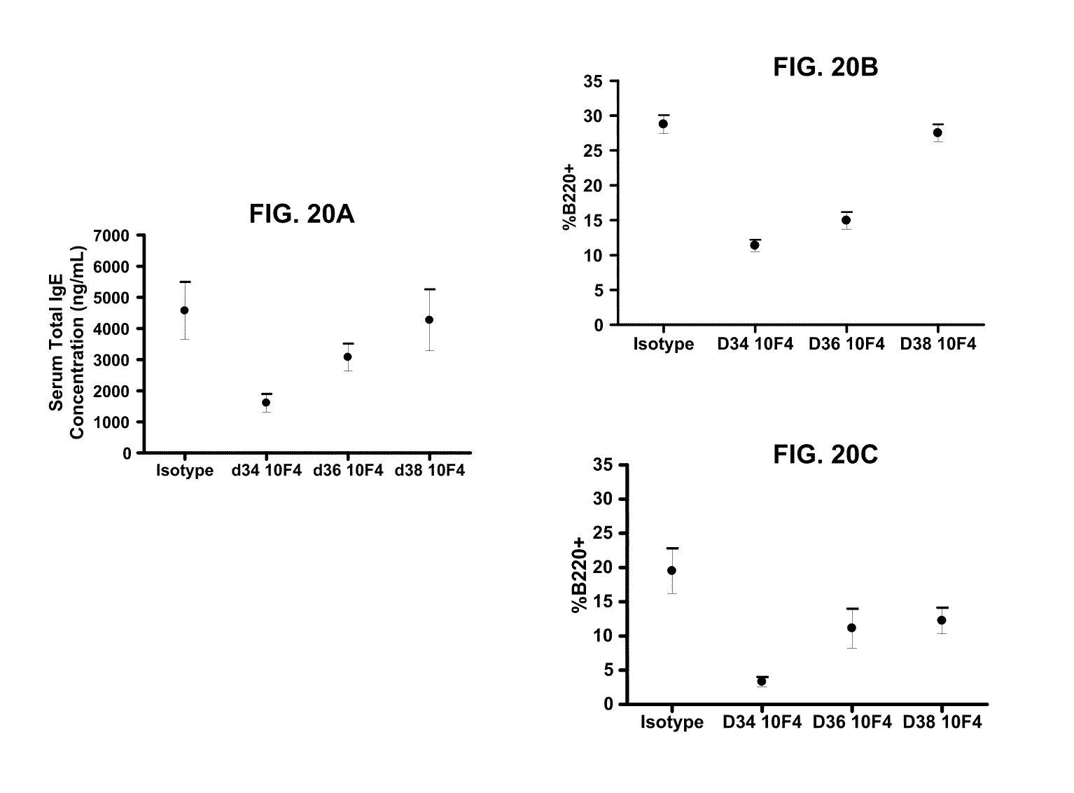

Serum IgE was detected as described in Example 4 using BD Biosciences Mouse OptEIA Total IgE ELISA Kit. As expected, total IgE levels were significantly elevated in mice treated with the isotype control antibody (Isotype) (FIG. 20A). In contrast, total IgE levels were significantly reduced in mice treated with 10F4 on day 34 (d34 10F4) or day 36 (d36 10F4) of OVA challenge (FIG. 20A). Total IgE levels in mice treated with 10F4 on day 38 (d38 10F4) of OVA challenge were similar to the total IgE levels in mice treated with the isotype control antibody (Isotype) (FIG. 20A). These data demonstrate that administration of a BLyS antagonist during the onset of allergen challenge (i.e., 1 or 3 days after the first allergen challenge) effectively reduced the allergen-induced increase in total IgE levels.

### Flow Cytometry

CD45R/B220+ cells were detected by flow cytometry as described in Example 4. The percent of B220+ B cells in the spleen is depicted in FIG. 20B for each treatment group and the percent of B220+ B cells in the lung is depicted in FIG. 20C for each treatment group. As shown in FIG. 20B, the percent of B220+ B cells in the spleen was significantly reduced in mice treated with 10F4 on day 34 (D34 10F4) and day 36 (D36 10F4) of OVA challenge as compared to mice treated with the isotype control antibody (Isotype) and mice treated with 10F4 on day 38 (D38 10F4) of OVA challenge. As shown in FIG. 20C, the percent of B220+ B cells in the lung was significantly reduced in mice treated with 10F4 on day 34 (D34 10F4), day 36 (D36 10F4), and day 38 (D38 10F4) of OVA challenge as compared to mice treated with the isotype control antibody (Isotype). These results confirm that treatment with anti-BLyS mAb 10F4 during the onset of allergen challenge (i.e., 1 or 3 days after the first allergen challenge) led to a significant reduction of B-cells in vivo.

The results of the experiments reflected in this example demonstrate that in vivo BLyS neutralization at the onset of allergen challenge can prevent, ameliorate, and/or treat allergen-induced allergic or inflammatory conditions of the lung. In particular, administration of a neutralizing anti-BLyS mAb 1 or 3 days after the first allergen challenge in a murine model of asthma significantly reduced the allergen-induced increase in total serum IgE levels and B-cells in the lung and spleen.

### Example 10

This example demonstrates that administration of a B Lymphocyte Stimulator (BLyS) antagonist can prevent, ameliorate, and/or treat chronic allergen-induced allergic or inflammatory conditions of the lung.

### Ovalbumin Sensitization and Challenge

Female BALB/c mice (8-10 weeks of age) were sensitized with intraperitoneal (i.p.) injections of 20 μg (0.1 ml of 200 m/ml in PBS) ovalbumin (OVA) (Sigma-Aldrich) every other day from day 1 through 19. Mice were challenged by nebulization with 10 mg/ml OVA in PBS for 40 minutes on days 33, 35, 37, 39, 54, 56, 58, 60, 73, 75, 77, 79, 93, 95, 97, and 99. The mice were terminated on day 100, after the last ovalbumin challenge. Control mice were sensitized and challenged with PBS and terminated on day 100. Upon termination, blood, lung, bronchoalveolar lavage (BAL) fluid, and spleen were removed from the animals. In addition, the blood was centrifuged and the serum was removed.

### 10F4 Treatment in OVA-Challenged Mice

To assess the ability of a B Lymphocyte Stimulator antagonist to prevent, treat or ameliorate chronic lung inflammation after repeated challenges with an antigen, mice were treated with 100 μg of monoclonal IgG1 hamster anti-mouse BLyS (clone 10F4) by i.p. injection on either days 34 and 41 (d34/41 10F4) (i.e., after the 4^{th} allergen challenge), on days 61 and 68 (d61/68 10F4) (i.e., after the 8^{th} allergen challenge), or on days 80 and 87 (D80/87 10F4) (i.e., after the 12^{th} allergen challenge). As a control, OVA-sensitized mice were treated with 100 μg of an isotype control antibody (purified Armenian hamster IgG1 (clone G235-2356; BD Biosciences)) by i.p. injection on either days 34 and 41 (d34/41 Iso) (i.e., after the 4^{th} allergen challenge), on days 61 and 68 (d61/68 Iso) (i.e., after the 8^{th} allergen challenge), or on days 80 and 87 (D80/87 Iso) (i.e., after the 12^{th} allergen challenge). As an additional control, one group of mice was left untreated (No Rx).

### Total IgE ELISA

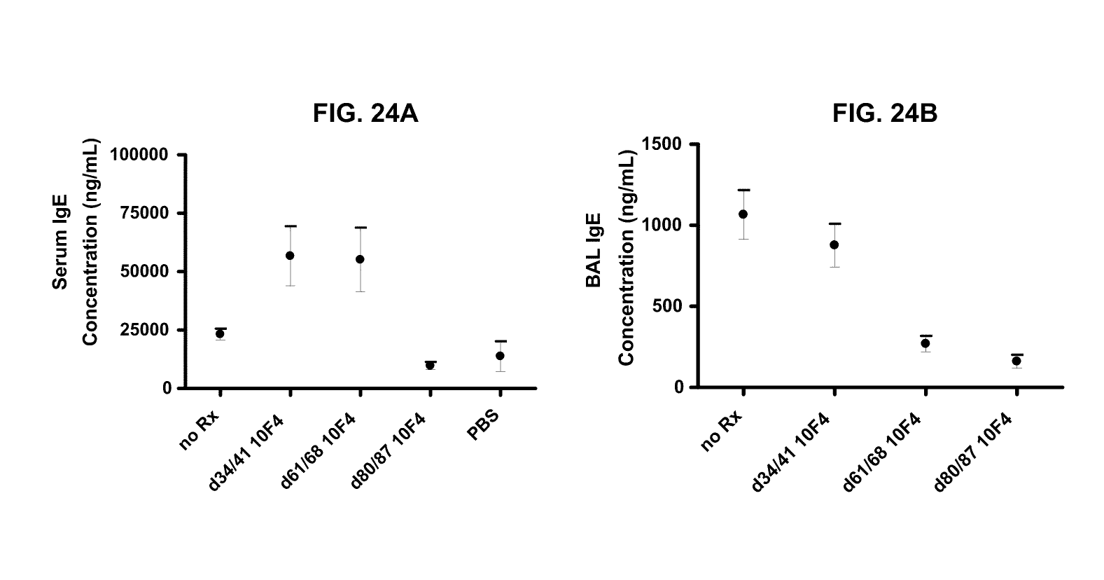

Total IgE in the serum and in the BAL fluid was detected as described in Example 4 using BD Biosciences Mouse OptEIA Total IgE ELISA Kit. As shown in FIG. 24A, total serum IgE levels were significantly reduced in mice treated with 10F4 on days 80 and 87 (d80/87 10F4) as compared to mice treated with 10F4 on days 34 and 41 (d34/41 10F4) or on days 61 and 68 (d61/68 10F4). As expected, low levels of total serum IgE were detected in mice sensitized and challenged with PBS (PBS) (FIG. 24A).

As shown in FIG. 24B, total BAL fluid IgE levels were significantly reduced in mice treated with 10F4 on days 61 and 68 (d61/68 10F4) and on days 80 and 87 (d80/87 10F4) as compared to untreated mice (No Rx). In addition, a modest decrease in total BAL fluid IgE levels was observed in mice treated with 10F4 on days 34 and 41 (d34/41 10F4) as compared to untreated mice (no Rx) (FIG. 24B).

These data demonstrate that treatment with a BLyS antagonist effectively reduced the allergen-induced increase in IgE levels in the serum and BAL fluid when administered after repeated antigen challenges.

### Flow Cytometry

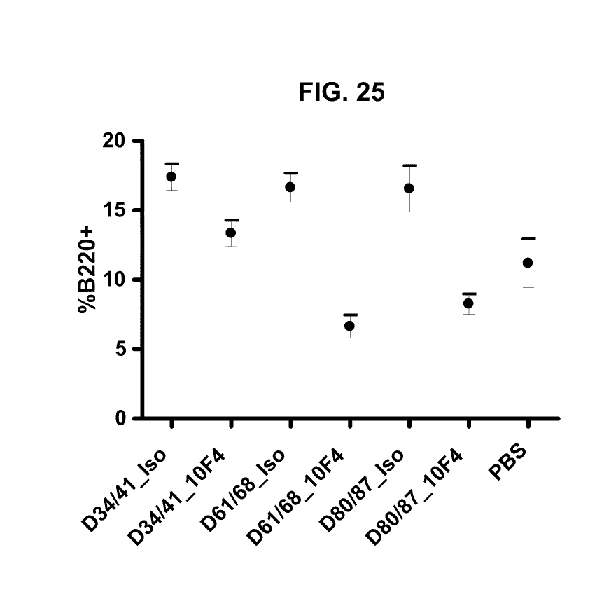

CD45R/B220+ cells in the lung were detected by flow cytometry as described in Example 4. As shown in FIG. 25, the percent of B220+ B cells in the lung was significantly reduced in mice treated with 10F4 on days 34 and 41 (d34/41 10F4), on days 61 and 68 (d61/68 10F4), and on days 80 and 87 (d80/87 10F4) as compared to the matched isotype control treated mice (d34/41 Iso, d61/68 Iso, and d80/87 Iso, respectively). These results confirm that treatment with anti-BLyS mAb 10F4 after repeated allergen challenges led to a significant reduction of B-cells in vivo. These data also demonstrate that the effect of treatment with a BLyS antagonist on reduction of B-cells is sustained after repeated subsequent allergen challenges with no further treatment.

### IL-13 ELISA

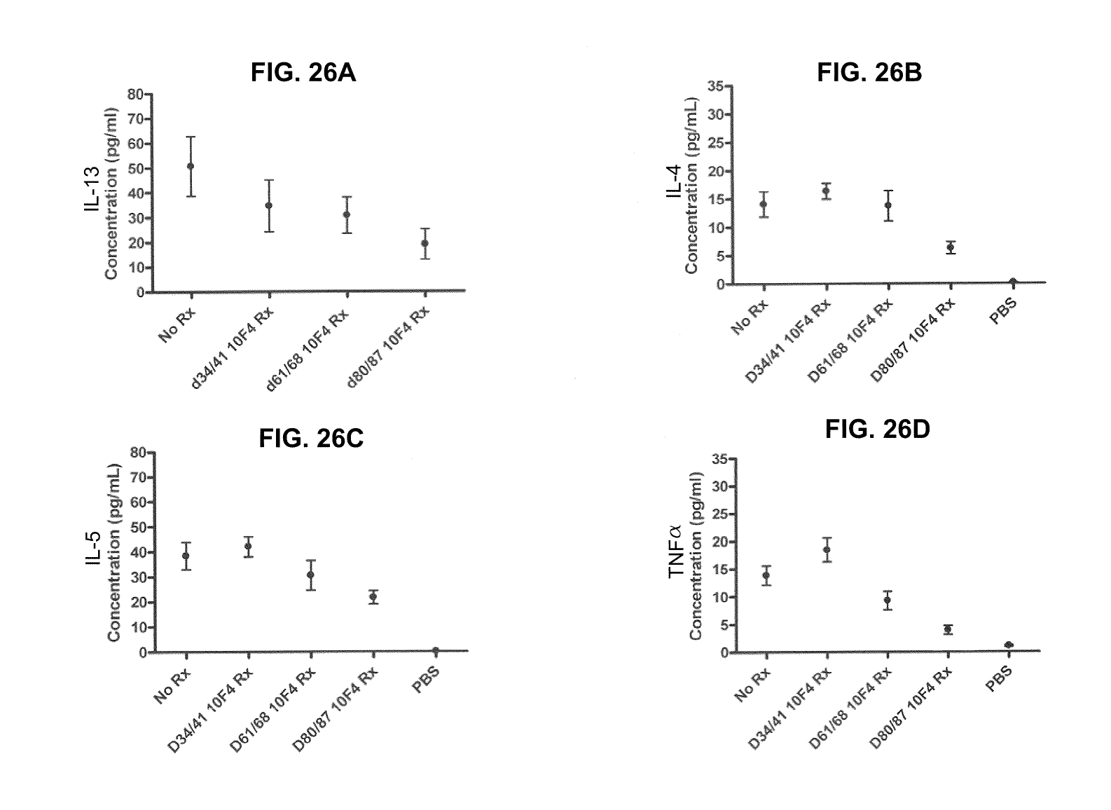

IL-13 levels were detected in BAL fluid using an IL-13 ELISA (eBioscience, cat. #88-7137-22) according to the manufacturer's protocol. As shown in FIG. 26A, IL-13 levels were significantly reduced in mice treated with 10F4 on days 34 and 41 (d34/41 10F4 Rx), on days 61 and 68 (d61/68 10F4 Rx), and on days 80 and 87 (d80/87 10F4 Rx) as compared to untreated mice (no Rx). These data demonstrate that treatment with a BLyS antagonist effectively reduced the allergen-induced increase in IL-13 levels in the BAL fluid when administered after repeated antigen challenges.

### IL-4, IL-5, and TNF-α ELISA

IL-4, IL-5, and TNF-α levels were detected in BAL fluid using a TH1/TH2 ELISA kit (MESO Scale, cat. #K15013C-1) according to the manufacturer's protocol.

IL-4 levels were significantly reduced in mice treated with 10F4 on days 80 and 87 (d80/87 10F4 Rx) as compared to untreated mice (no Rx) and mice treated with 10F4 on days 34 and 41 (d34/41 10F4 Rx) and on days 61 and 68 (d61/68 10F4 Rx) (FIG. 26B). IL-4 levels were significantly reduced in mice treated with 10F4 on days 80 and 87 (d80/87 10F4 Rx) as compared to untreated mice (no Rx) and mice treated with 10F4 on days 34 and 41 (d34/41 10F4 Rx) (FIG. 26C). A modest reduction in IL-4 levels was observed in mice treated with 10F4 on days 61 and 68 (d61/68 10F4 Rx), as compared to untreated mice (FIG. 26C). Similarly, TNF-α levels were significantly reduced in mice treated with 10F4 on days 80 and 87 (d80/87 10F4 Rx) as compared to untreated mice (no Rx) and mice treated with 10F4 on days 34 and 41 (d34/41 10F4 Rx), and a modest reduction in TNF-α levels was observed in mice treated with 10F4 on days 61 and 68 (d61/68 10F4 Rx), as compared to untreated mice (FIG. 26D). As expected, low levels of IL-4, IL-5, and TNF-α were detected in mice sensitized and challenged with PBS (PBS) (FIG. 26B-D).

These data demonstrate that treatment with a BLyS antagonist effectively reduced the allergen-induced increase in cytokines IL-4, IL-5, and TNF-α in the BAL fluid when administered after repeated antigen challenges.

### Lung Histology

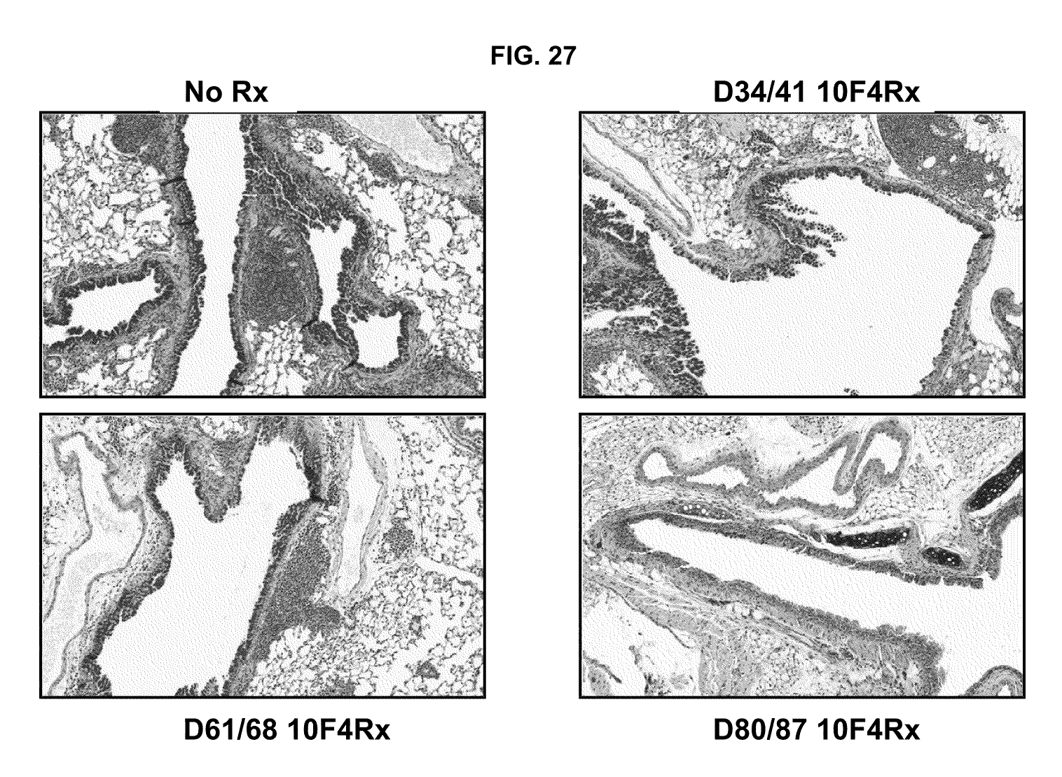

Lungs were analyzed by histology as described in Example 4. As shown in FIG. 27, mucus accumulation (red staining) and cellular infiltration (blue staining) was significantly reduced in mice treated with 10F4 on days 80 and 87 (d80/87 10F4Rx) as compared to untreated mice (No Rx) and mice treated with 10F4 on days 34 and 41 (d34/41 10F4Rx). In addition, a modest decrease in mucus accumulation (red staining) and cellular infiltration (blue staining) was observed in mice treated with 10F4 on days 61 and 68 (d61/68 10F4Rx) as compared to untreated mice (No Rx). These data demonstrate that treatment with a B Lymphocyte Stimulator antagonist after repeated allergen challenges inhibited allergen-induced increase in mucus accumulation, mucus-containing cells in the lung, or general cellular infiltration of immune cells in the lung.

The results of the experiments reflected in this example demonstrate that treatment with a BLyS antagonist effectively reduced the allergen-induced increase in (1) IgE levels in the serum and BAL fluid, (2) B-cell levels in the lung, (3) cytokines in the BAL fluid, and (4) mucus deposition and cellular infiltration in the lung, when administered after repeated antigen challenges. In view of the fact that the BLyS antagonist was administered prior to the final (i.e., the 13^{th}-16^{th}) antigen challenge, these data also demonstrate that treatment with a BLyS antagonist effectively protected against the allergen-induced increase in (1) IgE levels in the serum and BAL fluid, (2) B-cell levels in the lung, (3) cytokines in the BAL fluid, and (4) mucus deposition and cellular infiltration in the lung. These data also demonstrate that the effect of treatment with a BLyS antagonist is sustained after repeated subsequent allergen challenges with no further treatment. Thus, the results of the experiments reflected in this example demonstrate that in vivo BLyS neutralization after repeated challenges with an antigen can prevent, ameliorate, and/or treat allergen-induced allergic or inflammatory conditions of the lung.

### Example 11

This example demonstrates that in vivo B Lymphocyte Stimulator (BLyS) protein neutralization has a long-lasting effect in reducing total IgE levels in a murine model of asthma up to 201 days after administration.

### Ovalbumin Sensitization and Challenge

Female BALB/c mice (8-10 weeks of age) were sensitized with intraperitoneal (i.p.) injections of 20 μg (0.1 ml of 200 μg/ml in PBS) ovalbumin (OVA) (Sigma-Aldrich) every other day from day 1 through 19. Mice were challenged by nebulization with 10 mg/ml OVA in PBS for 40 minutes on days 33, 35, 37, 39, 47, 49, 51, 53, 61, 63, 65, 67, 120, 122, 124, 126, 152, 154, 156, 158, 249, 251, 253, and 255. Control mice were left untreated (naïve). Mice were terminated throughout the course of the experiment on days 40, 54, 68, 127, 159, and 256.

### 10F4 Treatment in OVA-Challenged Mice

To assess the long-term ability of a B Lymphocyte Stimulator antagonist to prevent, treat or ameliorate chronic lung inflammation, mice were treated with 100 μg of monoclonal IgG1 hamster anti-mouse BLyS (clone 10F4) by i.p. injection on day 34 (10F4), on days 34 and 41 (2× 10F4), or on days 34, 41, 48, and 55 (4× 10F4). Control mice were left untreated (No Rx or Unrx).

### Total IgE ELISA

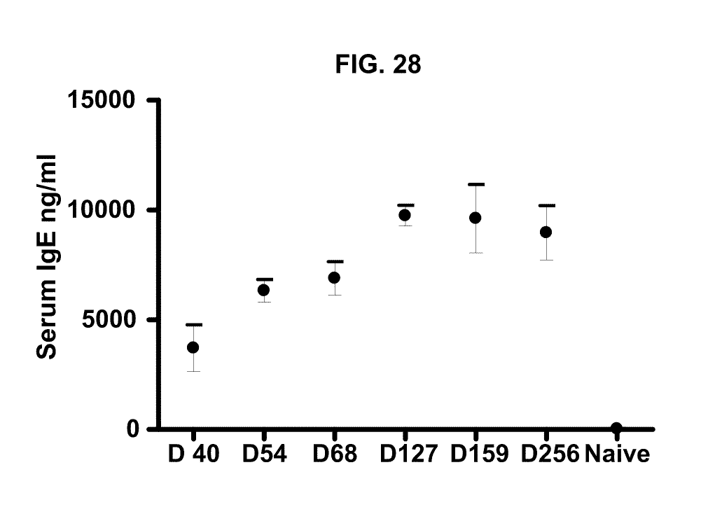

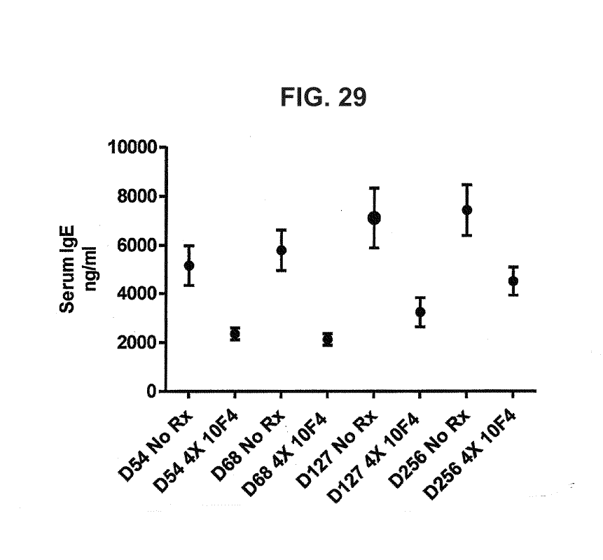

Serum IgE was detected as described in Example 4 using BD Biosciences Mouse OptEIA Total IgE ELISA Kit (FIG. 28) or as described in Example 8 using a total IgE ELISA developed by Human Genome Sciences, Inc. (Rockville, Md.) (FIG. 29). The total IgE ELISA developed by Human Genome Sciences, Inc. utilizes a different protocol and a different standard than the Mouse OptEIA Total IgE ELISA Kit obtained from BD Biosciences. Therefore, the results of the data presented in FIGS. 28 and 29 are not directly comparable because two different ELISAs were utilized to generate the data presented in FIGS. 28 and 29, respectively.

The levels of circulating total serum IgE in naïve mice and OVA-sensitized and challenged untreated mice (i.e., mice that did not receive 10F4) terminated on day 40, day 54, day 68, day 127, day 159, or day 256 are shown in FIG. 28. In untreated mice, total serum IgE levels increased throughout the course of the experiment as compared to naïve mice (FIG. 28).

As shown in FIG. 29, serum IgE levels were significantly decreased in mice that were treated four times with 10F4 (4× 10F4) and then terminated on day 54, day 68, day 127, or day 256, as compared to matched untreated OVA-sensitized and challenged mice (No Rx). Surprisingly, a significant decrease in serum IgE, as compared to the matched untreated OVA-sensitized and challenged mice, was observed in mice terminated 13 days, 72 days and 201 days after the last administration of 10F4 (on day 68, day 127 and day 256, respectively).

These data demonstrate that treatment with a BLyS antagonist effectively protected against the allergen-induced increase in IgE levels for up to 201 days after the last administration. In addition, these data demonstrate that the allergen-induced increase in total IgE levels was significantly reduced in mice treated four times with 10F4 (4× 10F4) even though the mice were challenged up to 16 more times with OVA following the final administration of an anti-BLyS antibody.

### Lung Infiltrate Analysis

Cellular infiltration was quantified in lung samples from mice terminated on day 68 as described in Example 4. The naïve control group (naïve) had an n=3, the untreated control group (Unrx) had an n=6, the group treated with 10F4 on days 34 and 41 (10F4 2X) had an n=6, and the group treated with 10F4 on days 34, 41, 48, and 55 (10F4 4X) had an n=5. The total area of each lung analyzed was 64±4.8 mm² (mean±SEM). An ANOVA with Dunnett's post-test was used for statistical analysis.

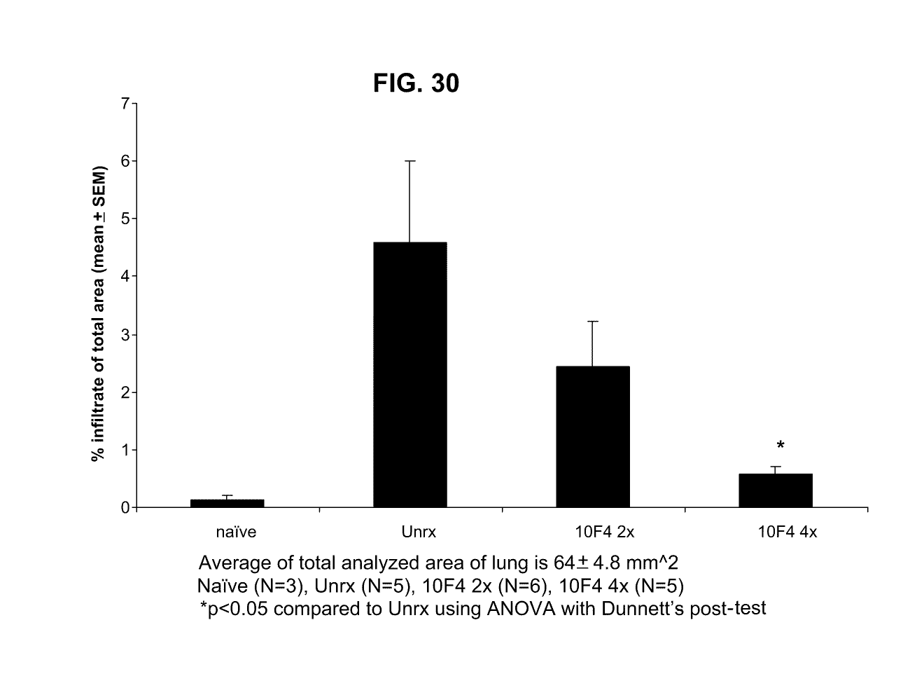

As shown in FIG. 30, cellular infiltration in the lungs was significantly elevated in untreated OVA-sensitized and challenged mice that were terminated on day 68 (Unrx) as compared to naïve mice that were terminated on day 68 (naïve). The allergen-induced increase in cellular infiltration observed in the untreated mice was significantly reduced in mice that were treated with 10F4 on days 34 and 41 and terminated on day 68 (10F4 2x) and in mice that were treated with 10F4 on days 34, 41, 48, and 55 and terminated on day 68 (10F4 4x) (FIG. 30). The * represents a p value <0.05 as compared to untreated mice (Unrx).

These data demonstrate that administration of four doses of a BLyS antagonist effectively reduced the allergen-induced increase in cellular infiltration in the lungs up to 13 days after the last dose.

### Lung Histology

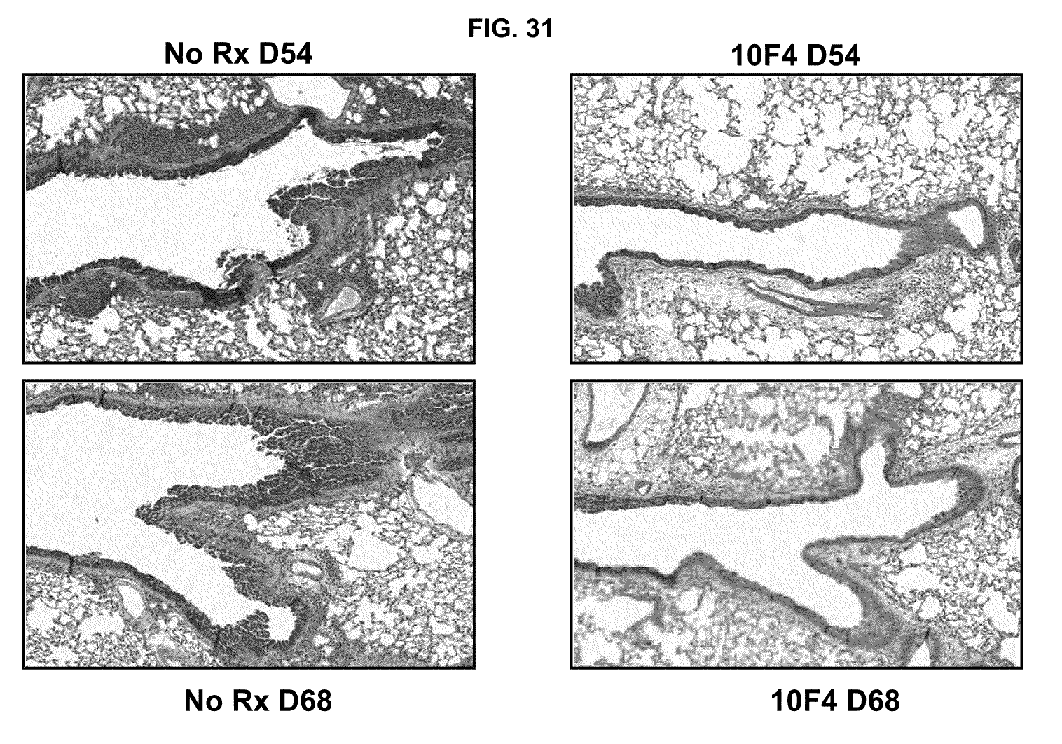

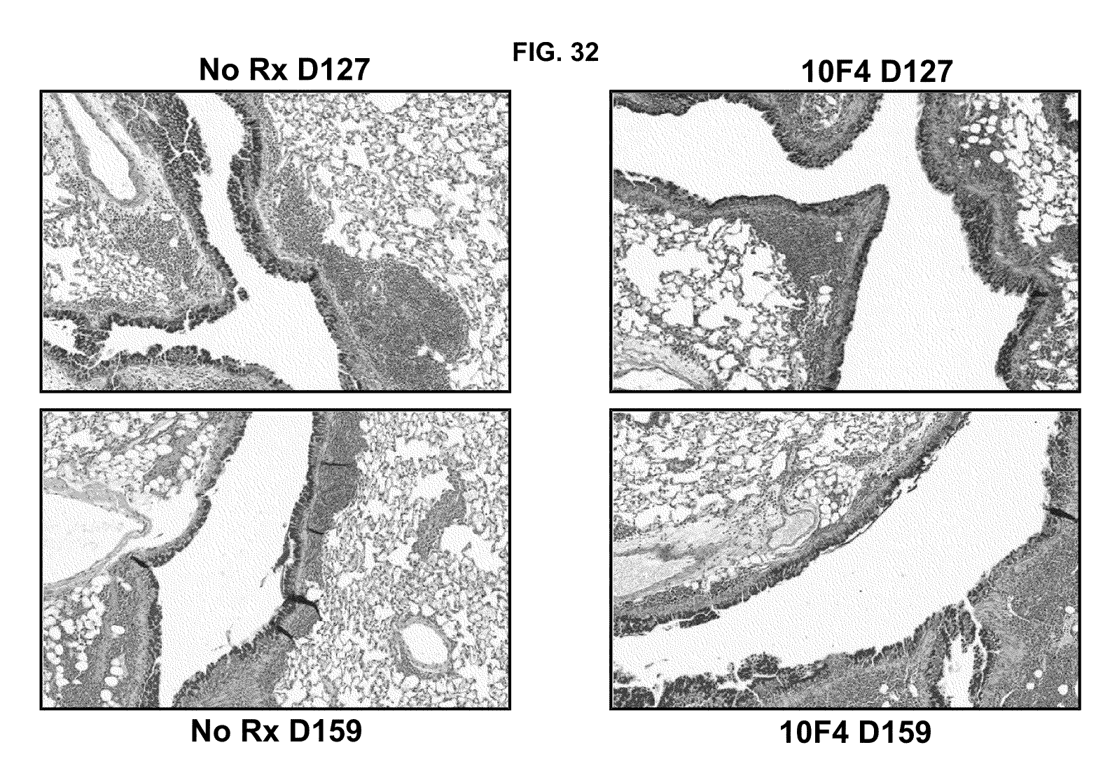

Lungs were analyzed by histology as described in Example 4. Consistent with the lung infiltrate analysis shown in FIG. 30, mucus accumulation (red staining) and cellular infiltration (blue staining) was significantly reduced in mice that were treated with 10F4 on days 34, 41, 48, and 55 and terminated either on day 54 (10F4 D54) or on day 68 (10F4 D68) as compared to matched untreated OVA-sensitized and challenged mice (No Rx D54 and No Rx D68, respectively) (FIG. 31). However, no significant decrease in mucus accumulation (red staining) and cellular infiltration (blue staining) was observed in mice that were treated with 10F4 on days 34, 41, 48, and 55 and terminated either on day 127 (10F4 D127) or on day 159 (10F4 D159) as compared to matched untreated OVA-sensitized and challenged mice (No Rx D127 and No Rx D159, respectively) (FIG. 32). These data demonstrate that administration of four doses of a BLyS antagonist inhibited allergen-induced increase in mucus accumulation, mucus-containing cells in the lung, or general cellular infiltration of immune cells in the lung up to 13 days after the last dose.

### B Cell Reconstitution

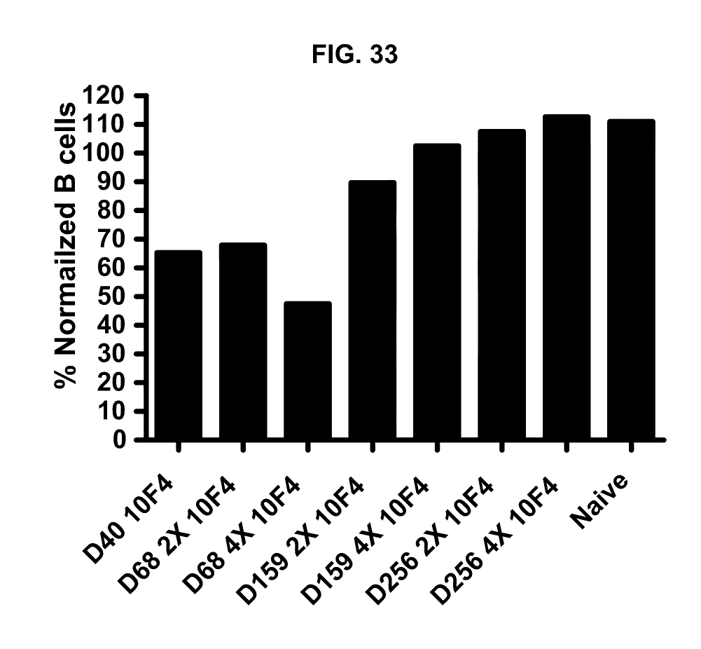

CD45R/B220+ cells in the spleen were detected by flow cytometry as described in Example 4 and the percentage of normalized B cells was calculated. As shown in FIG. 33, complete B cell reconstitution was observed in the spleen of mice treated four times with 10F4 by day 159.

The results of the experiments reflected in this example demonstrate that in vivo BLyS protein neutralization has a long-lasting effect in the prevention, treatment or amelioration of chronic lung inflammation. In particular, repeated administration of a neutralizing anti-BLyS monoclonal antibody (mAb) in a murine model of asthma significantly reduced total IgE levels in mice treated four times with 10F4 up to 201 days after administration. Thus, the results of the experiments reflected in this example demonstrate that repeated therapeutic administration of a neutralizing anti-BLyS mAb protected mice from the chronic recurrent ovalbumin challenge up to 201 days after the last dose. The chronic challenge model utilized in this experiment may replicate the pathology and serum IgE levels of a chronic asthma patient. Accordingly, the results of the experiment reflected in this example demonstrate that use of a BLyS antagonist, such as an anti-BLyS mAb, is a potential therapeutic for the treatment, prevention, and/or amelioration of an allergic or inflammatory condition of the lung such as asthma.

The experiments reflected in Examples 4 to 11 investigated the use of an anti-BLyS mAb (clone 10F4) as a potential therapeutic in a murine model of asthma. Asthma was induced by sensitization and challenge in an ovalbumin (OVA)-induced model in mice. Mice were chronically challenged with OVA and treated with anti-B1yS mAb either prophylactically (after OVA sensitization but before challenge) or therapeutically (after OVA sensitization and during challenge). The results of the experiments reflected in Examples 4 to 11 demonstrate that mice treated with anti-BLyS mAb had significantly reduced bronchial and systemic inflammation. Upon treatment, a significant decrease was observed in circulating total and OVA-specific IgE, as well as a marked reduction in mucus production and inflammatory lung infiltrates. Anti-BLyS treatment led not only to a decrease in circulating and splenic B cells but also downregulated expression of specific cytokines in the lung that have been routinely implicated in airway inflammation. These results suggest that intervention with an anti-BLyS antibody or a BLyS antagonist may be useful in the treatment of chronic asthma or other allergic or inflammatory conditions of the lung or respiratory system.

It will be clear that the invention may be practiced otherwise than as particularly described in the foregoing description and examples. Numerous modifications and variations of the invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

Further, the Sequence Listing submitted herewith is hereby incorporated by reference in its entirety. Additionally, the entire disclosure (including the specification, sequence listing, and drawings) of each of the following U.S. Provisional and Non-Provisional Patent Applications and International Patent Applications are herein incorporated by reference in their entireties: U.S. Provisional Applications 60/543,261 filed Feb. 11, 2004, 60/580,387 filed Jun. 18, 2004, 60/617,191 filed Oct. 12, 2004, 60/368,548 filed Apr. 1, 2002, 60/336,726 filed Dec. 7, 2001, 60/331,478 filed Nov. 16, 2001, 60/330,835 filed Oct. 31, 2001, 60/329,747 filed Oct. 18, 2001, and 60/329,508 filed Oct. 17, 2001, 60/225,628 filed Aug. 15, 2000, 60/227,008 filed Aug. 23, 2000, 60/234,338 filed Sep. 22, 2000, 60/240,806 filed Oct. 17, 2000, 60/250,020 filed Nov. 30, 2000, 60/276,248 filed Mar. 6, 2001, 60/293,499 filed May 25, 2001, 60/296,122 filed Jun. 7, 2001, 60/304,809 filed Jul., 13, 2001,60/122,388 filed Mar. 2, 1999, 60/124,097 filed Mar. 12, 1999, 60/126,599 filed Mar. 26, 2000, 60/127,598 filed Apr. 2, 1999, 60/130,412 filed Apr. 16, 1999, 60/130,696 filed Apr. 23, 1999, 60/131,278 filed Apr. 27, 1999, 60/131,673 filed Apr. 29, 1999, 60/136,784 filed May 28, 1999, 60/142,659 filed Jul. 6, 1999, 60/145,824 filed Jul. 27, 1999, 60/167,239 filed Nov. 24, 1999, 60/168,624 filed Dec. 3, 1999, 60/171,108 filed Dec. 16, 1999, 60/171,626 filed Dec. 23, 1999, 60/176,015 filed Jan. 14, 2000, and 60/036,100 filed Jan. 14, 1997; U.S. (Nonprovisional) patent application Ser. Nos. 10/739,042 filed Dec. 19, 2003, 10/735,865 filed Dec. 16, 2003, 10/270,487 filed Oct. 16, 2002, 09/929,493, filed Aug. 14, 2001, 09/588,947 filed Jun. 8, 2000, 09/589,285 filed Jun. 8, 2000, 09/589,286 filed Jun. 8, 2000, 09/589,287 filed Jun. 8, 2000, 09/589,288 filed Jun. 8, 2000, 09/507,968 filed Feb. 22, 2000, 09/255,794 filed Feb. 23, 1999, and 09/005,874 filed Jan. 12, 1998; and International Patent Applications PCT/US01/25549 filed Aug. 15, 2001, PCT/US00/04336, filed Feb. 22, 2000, and PCT/US96/17957, filed Oct. 25, 1996.

1. A method of treating, preventing or ameliorating asthma in a patient comprising administering to the patient an effective amount of an anti-B Lymphocyte Stimulator antibody, thereby treating, preventing or ameliorating asthma in the patient.

2. The method of claim 1, wherein the anti-B Lymphocyte Stimulator antibody comprises a first amino acid sequence comprising amino acid residues 1-123 of SEQ ID NO: 61 and a second amino acid sequence comprising amino acid residues 141-249 of SEQ ID NO: 61.

3. The method of claim 1, wherein the anti-B Lymphocyte Stimulator antibody is belimumab.

4. The method of claim 1, wherein the anti-B Lymphocyte Stimulator antibody is administered to the patient by intravenous injection, subcutaneous injection, or nebulization.

5. The method of claim 1, wherein the anti-B Lymphocyte Stimulator antibody is administered once every 1, 2, 4 or 6 months.

6. The method of claim 5, wherein the anti-B Lymphocyte Stimulator antibody is initially administered before the onset of symptoms of asthma in the patient.

7. The method of claim 6, wherein the symptoms of asthma are selected from the group consisting of wheezing; coughing; chest tightness; airflow obstruction;
bronchospasm; bronchoconstriction; and shortness of breath.

8. A method of treating, preventing or ameliorating an inflammatory condition of the lung or respiratory system in a patient comprising administering to the patient an effective amount of an anti-B Lymphocyte Stimulator antibody, thereby treating, preventing or ameliorating lung or respiratory system inflammation in the patient.

9. The method of claim 8, wherein the inflammatory condition is characterized by an increase in allergen-induced airway hyper responsiveness.

10. The method of claim 8, wherein the inflammatory condition is characterized by an increase in mucus-containing cells, mucus accumulation, or mucus production.

11. The method of claim 8, wherein the inflammatory condition is characterized by an increase in cellular infiltration in the lung.

12. The method of claim 11, wherein the increase in cellular infiltration is due to an increase in infiltration of immune cells into the lung.

13. The method of claim 12, wherein the immune cells are selected from the group consisting of B cells, T cells, eosinophils, neutrophils, mast cells, and other leukocytes.

14. The method of claim 8, wherein the inflammatory condition is selected from the group consisting of acute or chronic inflammation of the lung; pneumonia; emphysema; inflammatory lung injury; bronchiolitis obliterans; chronic bronchitis; pulmonary sarcoisosis; chronic obstructive pulmonary disease (COPD); interstitial lung disease; idiopathic pulmonary fibrosis; acute respiratory distress syndrome (ARDS); bronchiectasis; lung eosinophilia; interstitial fibrosis; cystic fibrosis; and chronic rhinosinusitis.

15. The method of claim 8, wherein the anti-B Lymphocyte Stimulator antibody comprises a first amino acid sequence comprising amino acid residues 1-123 of SEQ ID NO: 61 and a second amino acid sequence comprising amino acid residues 141-249 of SEQ ID NO: 61.

16. The method of claim 8, wherein the anti-B Lymphocyte Stimulator antibody is belimumab.

17. The method of claim 8, wherein the anti-B Lymphocyte Stimulator antibody is administered to the patient by intravenous injection, subcutaneous injection, or nebulization.

18. The method of claim 8, wherein the anti-B Lymphocyte Stimulator antibody is administered once every 1, 2, 4 or 6 months.

19. The method of claim 8, wherein the anti-B Lymphocyte Stimulator antibody is initially administered before the onset of symptoms of lung or respiratory system inflammation in the patient.

20. The method of claim 19, wherein the symptoms of lung or respiratory system inflammation are selected from the group consisting of wheezing; coughing; chest tightness; airflow obstruction; bronchospasm; bronchoconstriction; and shortness of breath.

21. A method of treating, preventing or ameliorating an allergic condition of the lung or respiratory system in a patient comprising administering to the patient an effective amount of an anti-B Lymphocyte Stimulator antibody, thereby treating, preventing or ameliorating the allergic condition of the lung or respiratory system in the patient.

22. The method of claim 21, wherein the allergic condition is selected from the group consisting of extrinsic allergic alveolitis (hypersensitivity pneumonitis); allergic bronchopulmonary aspergillosis; acute and chronic eosinophilic pneumonia; Churg-Strauss syndrome; and idiopathic hypereosinophilic syndrome.

23. A method of reducing total serum IgE levels or antigen-specific IgE levels in a patient suffering from asthma or an allergic or inflammatory condition of the lung or respiratory system comprising administering to the patient an effective amount of an anti-B Lymphocyte Stimulator antibody, thereby reducing total serum IgE levels or antigen-specific IgE levels in the patient.

24. A method of treating, preventing or ameliorating allergen-induced airway hyper responsiveness in a patient comprising administering to the patient an effective amount of an anti-B Lymphocyte Stimulator antibody, thereby treating, preventing or ameliorating allergen-induced airway hyper responsiveness in the patient.

25. A method of treating, preventing or ameliorating an allergen-induced increase in mucus-containing cells or mucus accumulation or production in the airway epithelium of a patient comprising administering to the patient an effective amount of an anti-B Lymphocyte Stimulator antibody, thereby treating, preventing or ameliorating an allergen-induced increase in mucus-containing cells or mucus accumulation or production in the airway epithelium of the patient.

26. A method of inhibiting or reducing allergen-induced cellular infiltration in the lungs of a patient diagnosed with an allergic or inflammatory condition of the lung or respiratory system comprising administering to the patient an effective amount of an anti-B Lymphocyte Stimulator antibody, thereby inhibiting or reducing allergen-induced cellular infiltration in the lungs of the patient.

27. The method of claim 26, wherein the increase in cellular infiltration is due to an increase in infiltration of immune cells into the lung.

28. The method of claim 27, wherein the immune cells are selected from the group consisting of B cells, T cells, eosinophils, neutrophils, mast cells, and other leukocytes.

29. The method of claim 26, wherein the inflammatory condition is selected from the group consisting of acute or chronic inflammation of the lung; pneumonia; emphysema; inflammatory lung injury; bronchiolitis obliterans; chronic bronchitis; pulmonary sarcoisosis; chronic obstructive pulmonary disease (COPD); interstitial lung disease; idiopathic pulmonary fibrosis; acute respiratory distress syndrome (ARDS); bronchiectasis; lung eosinophilia; interstitial fibrosis; cystic fibrosis; and chronic rhinosinusitis.

30. The method of claim 26, wherein the allergic condition is selected from the group consisting of extrinsic allergic alveolitis (hypersensitivity pneumonitis); allergic bronchopulmonary aspergillosis; acute and chronic eosinophilic pneumonia; Churg-Strauss syndrome; and idiopathic hypereosinophilic syndrome.

31. A method of treating, preventing or ameliorating an allergen-induced increase in cytokine production in the airway epithelium of a patient comprising administering to the patient an effective amount of an anti-B Lymphocyte Stimulator antibody, thereby treating, preventing or ameliorating an allergen-induced increase in cytokine production in the airway epithelium of the patient.

32. A method of promoting tolerance to an allergen in a patient comprising administering to the patient an effective amount of a B Lymphocyte Stimulator antagonist before, during, or after exposure to the allergen, thereby ameliorating or preventing symptoms of asthma or an allergic or inflammatory condition of the lung or respiratory system and promoting tolerance to the allergen in the patient.

33. The method of claim 32, wherein the B Lymphocyte Stimulator antagonist is selected from the group consisting of:
(a) a protein comprising the B Lymphocyte Stimulator binding domain of TACI;
(b) a protein comprising the B Lymphocyte Stimulator binding domain of BCMA;
(c) a protein comprising the B Lymphocyte Stimulator binding domain of BAFF-R;
(d) a B Lymphocyte Stimulator-binding peptide;
(e) a B Lymphocyte Stimulator peptibody;
(f) a B Lymphocyte Stimulator protein variant;
(g) an anti-B Lymphocyte Stimulator antibody; and
(h) an anti-B Lymphocyte Stimulator receptor antibody.

34. The method of claim 33, wherein the B Lymphocyte Stimulator antagonist is an anti-B Lymphocyte Stimulator antibody.

35. The method of claim 34, wherein the anti-B Lymphocyte Stimulator antibody comprises a first amino acid sequence comprising amino acid residues 1-123 of SEQ ID NO: 61 and a second amino acid sequence comprising amino acid residues 141-249 of SEQ ID NO: 61.

36. The method of claim 34, wherein the anti-B Lymphocyte Stimulator antibody is belimumab.

37. The method of claim 34, wherein the anti-B Lymphocyte Stimulator antibody binds a protein selected from the group consisting of:
(a) soluble B Lymphocyte Stimulator protein;
(b) membrane-bound B Lymphocyte Stimulator protein;
(c) the amino acid sequence of amino acid residues 1-285 of SEQ ID NO:2;
(d) the amino acid sequence of amino acid residues 134-285 of SEQ ID NO:2;
(e) a trimer of (d); and
(f) the amino acid sequence of a fragment of the polypeptide of SEQ ID NO:2; wherein the fragment is at least 30 amino acids in length and wherein the fragment is capable of stimulating B cell proliferation, differentiation, or survival.

38. The method of claim 32, wherein the B Lymphocyte Stimulator antagonist is administered to the patient by intravenous injection, subcutaneous injection, or nebulization.

39. The method of claim 32, wherein the B Lymphocyte Stimulator antagonist is administered once every 1, 2, 4 or 6 months.

40. The method of claim 39, wherein the B Lymphocyte Stimulator antagonist is initially administered before the onset of symptoms of asthma or an allergic or inflammatory condition of the lung or respiratory system in the patient.

41. The method of claim 40, wherein the symptoms of asthma or an allergic or inflammatory condition of the lung or respiratory system are selected from the group consisting of: wheezing; coughing; chest tightness; airflow obstruction; bronchospasm; bronchoconstriction; and shortness of breath.
